(19) United States
(12) Patent Application Publication
(10) Pub. No.: US 2024/0226052 A1
(43) Pub. Date: Jul. 11, 2024

(54) A SPECIFIC COMBINATION OF LIPIDS AND METHODS AND USES RELATED THERETO

(71) Applicant: HELSINGIN YLIOPISTO, Helsinki (FI)

(72) Inventors: Filip EKHOLM, Helsinki (FI); Helena BLAND, Helsinki (FI); Jan-Erik RAITANEN, Helsinki (FI); Jukka MOILANEN, Helsinki (FI); Riku PAANANEN, Helsinki (FI); Tuomo VIITAJA, Helsinki (FI)

(73) Assignee: HELSINGIN YLIOPISTO, Helsinki (FI)

(21) Appl. No.: 18/557,371

(22) PCT Filed: Apr. 29, 2022
(86) PCT No.: PCT/EP2022/061585

§ 371 Date: Oct. 26, 2023

(30) Foreign Application Priority Data
Apr. 30, 2021 (FI) 20215505

(51) Int. Cl. A61K 31/231

(52) U.S. Cl. CPC A61K 31/231

(57) ABSTRACT

The present invention relates to the fields of life sciences and medicine. Specifically, the invention relates to a composition comprising a specific combination of lipids and optionally one or more additives, and a method of preparing said composition. Furthermore, the present invention relates to the composition of the present invention for use as a medicament, for use in the treatment of dry eye disease and/or Meibomian gland dysfunction, and for use in alleviation of eye discomfort. Moreover, the present invention relates to a method of treating dry eye disease and/or Meibomian gland dysfunction, or alleviating eye discomfort. In addition, the present invention relates to a non-therapeutic or therapeutic method of retarding evaporation of water and use of the composition of the present invention for preventing evaporation of water.

### FIELD OF THE INVENTION

The present invention relates to the fields of life sciences and medicine. In more detail, the invention relates to a composition comprising a specific combination of lipids and optionally one or more additives, and a method of preparing said composition. Furthermore, the present invention relates to the composition of the present invention for use as a medicament, for use in the treatment of dry eye disease and/or Meibomian gland dysfunction, and for use in alleviation of eye discomfort. Moreover, the present invention relates to a method of treating dry eye disease and/or Meibomian gland dysfunction, or alleviating eye discomfort. In addition, the present invention relates to a non-therapeutic or therapeutic method of retarding the evaporation of water and use of the composition of the present invention to prevent evaporation of water.

### BACKGROUND OF THE INVENTION

Dry Eye Disease (DED) affects 300-500 million people on a global scale and constitutes a severe economic burden with annual managing costs of $55 billion in the United States alone. DED is the most common reason for seeking medical eye care and constitutes a significant public health concern, as well as a considerable societal economic burden. An underlying cause of DED is a dysfunction in the Meibomian glands which alters the tear film lipid layer (TFLL) composition and leads to tear film instability. While a substantial amount of research has been invested in understanding DED and delivering improved treatments, the most commonly used treatment is still ocular lubricants and artificial tears. These treatments focus on alleviating DED symptoms and are often associated with unsatisfactory results because they are unable to efficiently target the tear film instability defect. Other treatment options such as topical corticosteroids are used to treat the resulting ocular surface inflammation and are associated with severe side-effects in long-term use which is a pitfall when considering the chronic nature of some instances of DED. There is a dire need for a novel DED treatment strategy aimed at restoring the tear film stability.

Recently there has been a growing interest in more efficient management of Meibomian gland dysfunction (MGD) and DED (Jones, L., et al. 2017, The Ocular Surface, 15, 575-628). Meibomian glands produce the tear film lipid layer (TFLL), which is the outermost layer of the tear film covering the ocular surface. The TFLL is a unique biological membrane mainly comprising of ultra-long chained non-polar wax esters (WE) and cholesteryl esters (CE), with scarce amounts of more polar lipids such as O-acyl-o-hydroxy fatty acids (OAHFA) present (Brown, S., H., et al. 2013, Invest Ophthalmol Vis Sci, 54, 7417-7423; Lam, S., M., et al. 2014, J Lipid Res, 55, 299-306; Rohit, A., et al. 2014, Optometry and Vision Science, 91, 1384-1390). TFLL dysfunction associated with MGD and DED is presently considered to cause partial loss of TFLL evaporation resistance which leads to excess evaporation of tear fluid from the aqueous layer of the tear film and to destabilization of the tear film, resulting in dry eyes and DED (Craig, J., P., et al. 2017, The Ocular Surface, 15, 276-283).

There is a clear need for new effective and specific compositions and methods for treating and/or alleviating the symptoms of DED, MGD, and eye discomfort. In this regard, novel treatment strategies aimed at restoring tear film stability are in demand and central to the future success in this field.

### BRIEF DESCRIPTION OF THE INVENTION

The shortcomings of the prior art, including but not limited to, a lack of simple, safe, effective and low-cost compositions and methods for preventing water evaporation or treating and/or alleviating the symptoms of DED and/or MGD can be overcome with the present invention.

The objects of the invention, namely simple, safe, low-cost and effective compositions and methods for preventing or retarding water evaporation, restoring tear film stability or treating DED and/or MGD, are achieved by utilizing a specific combination of lipids. The composition of the present invention has a superior suppressive effect on water evaporation. Indeed, the evaporation resistance achieved with the composition is exceptional. This striking property means that the composition can be utilized to prevent or retard evaporation of water from any material and in any surrounding. Therefore, suitable applications of the composition of the present invention include but are not limited to; human beings, any animal and any material comprising water, and furthermore, treating disorders or preventing/retarding the evaporation of water in natural surroundings such as artificial lakes and reservoirs. The latter applications present an increasing challenge due to the global climate change.

The composition of the present invention can spread well on an aqueous surface such as the surface of the tear fluid and can alone form an effective anti-evaporative film. Furthermore, the composition of the present invention is very natural and enables use of natural lipids, or their structural analogues, alone in a specific combination.

The present invention is based on the idea of using a combination comprising fatty acid esters of a hydroxy fatty acid (FAHFA) (e.g. O-Acyl-w-hydroxy fatty acids) or structural analogues thereof and wax esters (or structural analogues thereof). Said combination self-assembles rapidly at the air-tear interface and forms an anti-evaporative barrier. The microscale structure and biophysical properties of the tailored lipid composition of the present invention reveal the superiority of the present invention over the prior art. The biophysical properties stem from the intrinsic properties of the composition as such and are not directly linked to the surrounding environment.

Synergistic and significantly enhanced effects can be obtained with the composition of the present invention when compared to FAHFAs, OAHFAs or wax esters alone.

Specifically, the present invention relates to a composition comprising a combination of a fatty acid ester of a hydroxy fatty acid (FAHFA) or a structural analogue thereof and a wax ester or a structural analogue thereof, and optionally one or more additives.

Also, the present invention relates to the compositions of the present invention for use as a medicament.

Furthermore, the present invention relates to the composition of the present invention for use in the treatment of dry eye disease and/or Meibomian gland dysfunction, or for use in alleviation of eye discomfort.

In addition, the present invention relates to a method of preparing the composition of the present invention, wherein the method comprises combining or mixing a FAHFA or a structural analogue thereof and a wax ester or a structural analogue thereof, and optionally one or more additives.

Furthermore, the present invention relates to a non-therapeutic or therapeutic method of preventing evaporation of water, wherein the method comprises applying the composition of the present invention on a surface to gain a decreased evaporation rate or to a material to gain a decreased evaporation rate.

The present invention relates to use of the composition of the present invention for preventing evaporation of water.

In addition, the present invention relates to a method of treating dry eye disease and/or Meibomian gland dysfunction, or alleviating eye discomfort, wherein the method comprises administering the composition of the present invention to the surface of an eye of a subject in need thereof.

Furthermore, the present invention relates to use of a FAHFA or a structural analogue thereof and a wax ester or a structural analogue thereof, and optionally one or more additives in the manufacture of a medicament for the treatment of dry eye disease or Meibomian gland dysfunction or for the alleviation of eye discomfort.

The present invention relates also to novel branched wax esters and methods for their preparation.

The objects of the invention are achieved by compositions, uses and methods characterized by what is stated in the independent claims. The preferred embodiments of the invention are disclosed in the dependent claims.

Other objects, details and advantages of the present invention will become apparent from the following drawings, detailed description and examples.

### BRIEF DESCRIPTION OF THE DRAWINGS

FIG. 1 reveals surface pressure (1A) and surface potential (1B) isotherms of 20-(oleoyloxy)eicosanoic acid (20-OAHFA):arachidyl oleate (AO)-mixtures, with corresponding Brewster angle microscopy images (1C). Results are shown as a function of area/molecule (insets). In addition, a schematic representation of the molecular organization of the films is shown with 20-OAHFA molecules depicted in orange (darker grey) and AO molecules in yellow (lighter grey) (with oxygen atoms shown in black) (1D). Selected images correspond to the following conditions: (i) mixed liquid monolayer of 20-OAHFA and AO, (ii) collapse of AO on the monolayer surface, (iii) coexistence of gas and liquid monolayer phases, and (iv) formation of solid monolayer domains by 20-OAHFA with overlying AO.

FIG. 2 shows evaporation resistance, on the seconds/centimeter (s/cm) scale, of 20-OAHFA:AO-mixtures as a function of area/OAHFA.

FIG. 3 shows surface pressure (3A) and surface potential (3B) isotherms of 20-OAHFA:behenyl oleate (BO)-mixtures, with corresponding Brewster angle microscopy images of the mixtures (3C). Schematic representation of the molecular organization of the 20-OAHFA:BO mixed films during compression is shown in with 20-OAHFA molecules depicted in orange (darker grey) and BO molecules in yellow (lighter grey) (with oxygen atoms shown in black) (3D). Selected images correspond to the following conditions, (i, ii) formation of mixed solid monolayer domains of 20-OAHFA:BO, (iii) excess BO not mixed in the monolayer remains as solid aggregates, and (iv) solid mixed monolayer with excess BO aggregates collapsed on the monolayer surface.

FIG. 4 shows evaporation resistance of 20-OAHFA:BO-mixtures, on the s/cm scale, at different areas per molecule, as a function of film composition (fraction of 20-OAHFA).

FIG. 5 shows the evaporation resistance, on the second/centimeter (s/cm) scale as a function of the area/molecule, of the following 1:1 mixtures: 18-(oleoyloxy)stearic acid (18:0/18:1 OAHFA):behenyl behenoate (BB); (21Z)-29-(oleoyloxy)nonacos-21-enoic acid (29:1/18:1 OAHFA):BO; 18:0/18:1-OAHFA:BO; and 18:0/18:1-OAHFA:arachidyl laurate (AL). The combinations of 18:0/18:1-OAHFA and BO and 18:0/18:1-OAHFA and AL showed improved evaporation resistance compared to the individual components at all areas per molecule, but the values for mean molecular areas below 10 Å² were too high to be determined without recalibration of the instrument. The combinations of 18:0/18:1 OAHFA and BB and 29:1/18:1 OAHFA:BO did not show improved evaporation resistance.

### DETAILED DESCRIPTION OF THE INVENTION

The tear film consists of two distinct layers, the aqueous layer and the TFLL, which is considered to act as a barrier to evaporation of water from the underlying aqueous layer. The loss of this evaporation resistant function leads to drying of the eyes in the majority of DED-cases, which may further cause inflammation and ocular surface damage.

In the present invention, the inventors have developed synthetic protocols for the synthesis of an extensive library of TFLL FAHFAs and wax esters, and, their structural analogues. Using this library, the inventors have identified mixtures of the key lipid species that combine exceptionally high evaporation resistance with effective spreading on the aqueous interface, which is in line with the functioning principle of an intact TFLL. In more detail, the lipids need to spread rapidly and cover the entire aqueous tear film surface as the eye is opened, and, the film formed by the lipids needs to have a condensed structure that prevents or retards the passage of water molecules through it. The result of the present invention are compositions of FAHFAs and wax esters which form an evaporation resistant barrier on an aqueous interface under physiological conditions. Administering these lipid compositions on the ocular surface represents a unique and highly promising treatment for DED and/or eye discomfort. Moreover, because the biophysical properties stem from the intrinsic properties of the mixture as such and are not directly linked to the surrounding environment—these mixtures can likewise be used to prevent the evaporation of water from materials and other surroundings e.g. artificial lakes and water reservoirs. The latter task represents an increasing challenge due to the global climate change.

As used herein, “physiological conditions”, will take its usual meaning in the art, namely indicating the conditions that the skilled person would normally expect at the ocular surface of a subject, such as a human or animal (e.g. a human). For the avoidance of doubt, physiological conditions at the ocular surface of a human or animal may be a temperature of about 35° C., a surface pressure of about 27 to 31 mN/m and a pH of about 7.0 to 7.3. References to the physical state of lipids (i.e. FAHFAs and/or wax esters) at (or under) physiological conditions may particularly be understood to indicate the physical state (i.e. liquid or solid) of the lipid in question at a temperature of about 35° C. and atmospheric pressure.

The present invention concerns a composition comprising or consisting of i) lipids such as a combination of two different types of lipids, e.g. a polar lipid and a non-polar lipid, and optionally ii) one or more additives. In one embodiment the composition comprises or consists of i) a combination of a polar lipid selected from the group consisting of FAHFAs and structural analogues thereof, and a non-polar lipid selected from the group consisting of wax esters and structural analogues thereof, and optionally ii) one or more additives. In one embodiment the only lipids of the composition are one or more FAHFAs or structural analogues thereof (such as OAHFAs) and one or more wax esters (i.e. esters of a fatty acid and a fatty alcohol) or structural analogues thereof.

The present invention further concerns a composition comprising or consisting of a combination of a FAHFA or a structural analogue thereof and a wax ester or a structural analogue thereof, and optionally one or more additives. In one embodiment, these compositions do not comprise any further FAHFA (including structural analogues thereof) or wax ester (including structural analogues thereof) components.

In one embodiment, evaporation resistance of the composition is more than 1 s/cm, more than 2 s/cm or more than 3 s/cm. In one embodiment the evaporation resistance value can be as high as possible such as more than 5 s/cm, more than 9 s/cm, more than 10 s/cm, more than 13 s/cm, more than 15 s/cm, more than 20 s/cm, more than 25 s/cm or even more than 30 s/cm. More particularly, the evaporation resistance of the composition is higher than that of the natural tear forming lipid layer, which has been reported as 9-13 s/cm. Thus, the evaporation resistance of the composition is preferably more than 15 s/cm (for example more than 20 s/cm, such as from 15 to 30 s/cm (e.g. from 15 to 20 s/cm). In particular, such evaporation resistance values are achieved at an average mean molecular area of from about 2 to about 10 Å², such as from about 2 to about 5 Å² (e.g. 2-3 Å²).

As used herein “evaporation resistance” refers to the ability of the composition to prevent evaporation of water, wherein optionally the composition is on or above a surface of a material or agent to be prevented from said evaporation. It may be defined as r=Δc/J, where Δc is the water vapor concentration difference driving evaporation and J is the evaporative flux from the underlying aqueous phase defined as J=(dn/dt)/A, where n is the amount of water evaporating, t is the time, and A is the area of the surface. The evaporation resistance of a composition is a property of the lipid film residing on top or above an aqueous surface, independent of the measurement method and conditions of the measurement, and can be measured by any method known to a person skilled in the art, e.g. as described in chapter 1.2.4 of the examples part of the present disclosure or as described in Langmuir et al. (Langmuir, I. and Schaefer, V., J. 1943, J. Franklin Inst., Vol. 235, 119-162) In particular, the evaporation resistance of the compositions may be determined by:
1. measuring the evaporative flux from an aqueous surface in the absence of the composition (J_{w});
2. applying the composition to the aqueous surface and measuring the evaporative flux from the surface with the composition present (J_{f});
3. determining the evaporation resistance of the composition (rₘ) according to the equation rₘ=Δc(1/J_{f}−1/J_{w}),
   wherein Δc=the water vapor concentration difference driving evaporation.

Δc can be determined by methods known to the skilled person, but may be taken as 3.4±0.7·10⁻⁵ g·cm⁻³ for a water surface temperature of 30-35° C. at atmospheric pressure.

As described herein, evaporation resistance may be expressed in units of seconds/centimeter (s/cm). This may be related to a percentage reduction in evaporation rate at the ocular surface using the model developed by Cerretani et al. (Cerretani, C. F.; Ho, N. H.; Radke, C. J., Water-Evaporation Reduction by Duplex Films: Application to the Human Tear Film, Adv. Colloid Interface Sci. 2013, 197-198, 33-57.). Using this model, an evaporation resistance of 2 s/cm would reduce the evaporation rate at the ocular surface by 33-50%, while values such as 5 s/cm would correspond to a 60-80% reduction when a person is standing still or walking.

In one embodiment, a two-lipid-composition of a FAHFA, such as an OAHFA, and a wax ester results in an exceptionally high evaporation resistance. The evaporation resistance is the result of complex interactions between FAHFAs and other specific lipids, namely wax esters, of the composition of the present invention. The present disclosure is able to link evaporation resistance to the very specific tightly packed condensed lipid structures.

The carbon chain length of one or more FAHFAs or analogues thereof suitable for the composition of the present invention can vary. In one embodiment, there is no maximum carbon length. In one embodiment the carbon chain length of one or more FAHFAs or structural analogues thereof is C15-C100, C19-C72, C20-C55, C20-C50, C20-C40, C20-C35, C20-C25, C25-C45, C25-C40, C25-C35 or C25-C30. In one embodiment the carbon chain length of one or more FAHFAs or structural analogues thereof is C20, C21, C22, C23, C24, C25, C26, C27, C28, C29, C30, C31, C32, C33, C34, C35, C36, C37, C38, C39, C40, C41, C42, C43, C44, C45, C46, C47, C48, C49, C50, C51, C52, C53, C54 or C55.

In one embodiment, the FAHFA or a structural analogue thereof has the following formula (I)

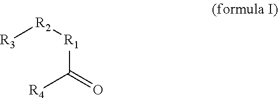

wherein
R₁ is a carbon atom, an oxygen atom or a nitrogen atom;
R₂ is a linear or branched C9-C50 alkyl, alkenyl or alkynyl chain, or a structural analogue thereof;
R₃ is a carboxyl, hydroxyl, amine, phosphate or silyl ether;
R₄ is a linear or branched C9-C50 alkyl, alkenyl or alkynyl chain, or a structural analogue thereof.

In one embodiment, R₁ is an oxygen atom in Formula I. When R₁ in Formula I is an oxygen atom, the compositions of the present invention remain stable long enough but still decompose naturally.

In one embodiment, one or more FAHFAs are selected from the group comprising or consisting of O-Acyl-w-hydroxy fatty acids (OAHFAs).

In one embodiment, one or more FAHFAs or OAHFAs are selected from the group comprising or consisting of oleic acid based fatty acid esters, palmitoleic acid based fatty acid esters, myristoleic acid based fatty acid esters, lauric acid based fatty acid esters, paullinic acid based fatty acid esters, gondoic acid based fatty acid esters, erucic acid based fatty acid esters, nervonic acid based fatty acid esters, linoleic acid based fatty acid esters, and linolenic acid based fatty acids; and/or structural analogues thereof. In one embodiment, one or more FAHFAs or OAHFAs are selected from the group comprising or consisting of oleic acid based fatty acid esters, palmitoleic acid based fatty acid esters, linoleic acid based fatty acid esters, and linolenic acid based fatty acids; and/or structural analogues thereof

In one embodiment, one or more FAHFAs or OAHFAs are selected from the group comprising or consisting of oleic acid based fatty acid esters. In one embodiment, one or more FAHFAs or OAHFAs are selected from the group comprising or consisting of 12-oleoyloxy-dodecanoic acid, 13-oleoyloxy-tridecanoic acid, 14-oleoyloxy-tetradecanoic acid, 15-oleoyloxy-pentadecanoic acid, 16-oleoyloxy-hexadecanoic acid, 17-oleoyloxy-heptadecanoic acid, 18-oleoyloxy-octadecanoic acid, 19-oleoyloxy-nonadecanoic acid, 20-oleoyloxy-eicosanoic acid, 21-oleoyloxy-heneicosanoic acid, 22-oleoyloxy-docosanoic acid, 23-oleoyloxy-tricosanoic acid, 24-oleoyloxy-tetracosanoic acid, 25-oleoyloxy-pentacosanoic acid, 26-oleoyloxy-hexacosanoic acid, 27-oleoyloxy-heptacosanoic acid, 28-oleoyloxy-octacosanoic acid, 29-oleoyloxy-nonacosanoic acid, 30-oleoyloxy-triacontanoic acid, 31-oleoyloxy-hentriacontanoic acid, 32-oleoyloxy-dotriacontanoic acid, 33-oleoyloxy-tritriacontanoic acid, 34-oleoyloxy-tetratriacontanoic acid, 35-oleoyloxy-pentatriacontanoic acid, 36-oleoyloxy-hexatriacontanoic acid, 37-oleoyloxy-heptatriacontanoic acid, 38-oleoyloxy-octatriacontanoic acid, 39-oleoyloxy-nonatriacontanoic acid and 40-oleoyloxy-tetracontanoic acid.

In one embodiment, one or more FAHFAs or OAHFAs are selected from the group comprising or consisting of palmitoleic acid based fatty acid esters. In one embodiment, one or more FAHFAs or OAHFAs are selected from the group comprising or consisting of 12-palmitoleoyloxy-dodecanoic acid, 13-palmitoleoyloxy-tridecanoic acid, 14-palmitoleoyloxy-tetradecanoic acid, 15-palmitoleoyloxy-pentadecanoic acid, 16-palmitoleoyloxy-hexadecanoic acid, 17-palmitoleoyloxy-heptadecanoic acid, 18-palmitoleoyloxy-octadecanoic acid, 19-palmitoleoyloxy-nonadecanoic acid, 20-palmitoleoyloxy-eicosanoic acid, 21-palmitoleoyloxy-heneicosanoic acid, 22-palmitoleoyloxy-docosanoic acid, 23-palmitoleoyloxy-tricosanoic acid, 24-palmitoleoyloxy-tetracosanoic acid, 25-palmitoleoyloxy-pentacosanoic acid, 26-palmitoleoyloxy-hexacosanoic acid, 27-palmitoleoyloxy-heptacosanoic acid, 28-palmitoleoyloxy-octacosanoic acid, 29-palmitoleoyloxy-nonacosanoic acid, 30-palmitoleoyloxy-triacontanoic acid, 31-palmitoleoyloxy-hentriacontanoic acid, 32-palmitoleoyloxy-dotriacontanoic acid, 33-palmitoleoyloxy-tritriacontanoic acid, 34-palmitoleoyloxy-tetratriacontanoic acid, 35-palmitoleoyloxy-pentatriacontanoic acid, 36-palmitoleoyloxy-hexatriacontanoic acid, 37-palmitoleoyloxy-heptatriacontanoic acid, 38-palmitoleoyloxy-octatriacontanoic acid, 39-palmitoleoyloxy-nonatriacontanoic acid and 40-palmitoleoyloxy-tetracontanoic acid.

In one embodiment, one or more FAHFAs or OAHFAs are selected from the group comprising or consisting of myristoleic acid based fatty acid esters. In one embodiment, one or more FAHFAs or OAHFAs are selected from the group comprising or consisting of 12-myristoleoyloxy-dodecanoic acid, 13-myristoleoyloxy-tridecanoic acid, 14-myristoleoyloxy-tetradecanoic acid, 15-myristoleoyloxy-pentadecanoic acid, 16-myristoleoyloxy-hexadecanoic acid, 17-myristoleoyloxy-heptadecanoic acid, 18-myristoleoyloxy-octadecanoic acid, 19-myristoleoyloxy-nonadecanoic acid, 20-myristoleoyloxy-eicosanoic acid, 21-myristoleoyloxy-heneicosanoic acid, 22-myristoleoyloxy-docosanoic acid, 23-myristoleoyloxy-tricosanoic acid, 24-myristoleoyloxy-tetracosanoic acid, 25-myristoleoyloxy-pentacosanoic acid, 26-myristoleoyloxy-hexacosanoic acid, 27-myristoleoyloxy-heptacosanoic acid, 28-myristoleoyloxy-octacosanoic acid, 29-myristoleoyloxy-nonacosanoic acid, 30-myristoleoyloxy-triacontanoic acid, 31-myristoleoyloxy-hentriacontanoic acid, 32-myristoleoyloxy-dotriacontanoic acid, 33-myristoleoyloxy-tritriacontanoic acid, 34-myristoleoyloxy-tetratriacontanoic acid, 35-myristoleoyloxy-pentatriacontanoic acid, 36-myristoleoyloxy-hexatriacontanoic acid, 37-myristoleoyloxy-heptatriacontanoic acid, 38-myristoleoyloxy-octatriacontanoic acid, 39-myristoleoyloxy-nonatriacontanoic acid and 40-myristoleoyloxy-tetracontanoic acid.

In one embodiment, one or more FAHFAs or OAHFAs are selected from the group comprising or consisting of lauric acid based fatty acid esters. In one embodiment, one or more FAHFAs or OAHFAs are selected from the group comprising or consisting of 12-dodecanoyloxy-dodecanoic acid, 13-dodecanoyloxy-tridecanoic acid, 14-dodecanoyloxy-tetradecanoic acid, 15-dodecanoyloxy-pentadecanoic acid, 16-dodecanoyloxy-hexadecanoic acid, 17-dodecanoyloxy-heptadecanoic acid, 18-dodecanoyloxy-octadecanoic acid, 19-dodecanoyloxy-nonadecanoic acid, 20-dodecanoyloxy-eicosanoic acid, 21-dodecanoyloxy-heneicosanoic acid, 22-dodecanoyloxy-docosanoic acid, 23-dodecanoyloxy-tricosanoic acid, 24-dodecanoyloxy-tetracosanoic acid, 25-dodecanoyloxy-pentacosanoic acid, 26-dodecanoyloxy-hexacosanoic acid, 27-dodecanoyloxy-heptacosanoic acid, 28-dodecanoyloxy-octacosanoic acid, 29-dodecanoyloxy-nonacosanoic acid, 30-dodecanoyloxy-triacontanoic acid, 31-dodecanoyloxy-hentriacontanoic acid, 32-dodecanoyloxy-dotriacontanoic acid, 33-dodecanoyloxy-tritriacontanoic acid, 34-dodecanoyloxy-tetratriacontanoic acid, 35-dodecanoyloxy-pentatriacontanoic acid, 36-dodecanoyloxy-hexatriacontanoic acid, 37-dodecanoyloxy-heptatriacontanoic acid, 38-dodecanoyloxy-octatriacontanoic acid, 39-dodecanoyloxy-nonatriacontanoic acid and 40-dodecanoyloxy-tetracontanoic acid.

In one embodiment, one or more FAHFAs or OAHFAs are selected from the group comprising or consisting of paullinic acid based fatty acid esters. In one embodiment, one or more FAHFAs or OAHFAs are selected from the group comprising or consisting of 12-(eicos-13-enoyloxy)-dodecanoic acid, 13-(eicos-13-enoyloxy)-tridecanoic acid, 14-(eicos-13-enoyloxy)-tetradecanoic acid, 15-(eicos-13-enoyloxy)-pentadecanoic acid, 16-(eicos-13-enoyloxy)-hexadecanoic acid, 17-(eicos-13-enoyloxy)-heptadecanoic acid, 18-(eicos-13-enoyloxy)-octadecanoic acid, 19-(eicos-13-enoyloxy)-nonadecanoic acid, 20-(eicos-13-enoyloxy)-eicosanoic acid, 21-(eicos-13-enoyloxy)-heneicosanoic acid, 22-(eicos-13-enoyloxy)-docosanoic acid, 23-(eicos-13-enoyloxy)-tricosanoic acid, 24-(eicos-13-enoyloxy)-tetracosanoic acid, 25-(eicos-13-enoyloxy)-pentacosanoic acid, 26-(eicos-13-enoyloxy)-hexacosanoic acid, 27-(eicos-13-enoyloxy)-heptacosanoic acid, 28-(eicos-13-enoyloxy)-octacosanoic acid, 29-(eicos-13-enoyloxy)-nonacosanoic acid, 30-(eicos-13-enoyloxy)-triacontanoic acid, 31-(eicos-13-enoyloxy)-hentriacontanoic acid, 32-(eicos-13-enoyloxy)-dotriacontanoic acid, 33-(eicos-13-enoyloxy)-tritriacontanoic acid, 34-(eicos-13-enoyloxy)-tetratriacontanoic acid, 35-(eicos-13-enoyloxy)-pentatriacontanoic acid, 36-(eicos-13-enoyloxy)-hexatriacontanoic acid, 37-(eicos-13-enoyloxy)-heptatriacontanoic acid, 38-(eicos-13-enoyloxy)-octatriacontanoic acid, 39-(eicos-13-enoyloxy)-nonatriacontanoic acid and 40-(eicos-13-enoyloxy)-tetracontanoic acid.

In one embodiment, one or more FAHFAs or OAHFAs are selected from the group comprising or consisting of gondoic acid based fatty acid esters. In one embodiment, one or more FAHFAs or OAHFAs are selected from the group comprising or consisting of 12-(eicos-11-enoyloxy)-dodecanoic acid, 13-(eicos-11-enoyloxy)-tridecanoic acid, 14-(eicos-11-enoyloxy)-tetradecanoic acid, 15-(eicos-11-enoyloxy)-pentadecanoic acid, 16-(eicos-11-enoyloxy)-hexadecanoic acid, 17-(eicos-11-enoyloxy)-heptadecanoic acid, 18-(eicos-11-enoyloxy)-octadecanoic acid, 19-(eicos-11-enoyloxy)-nonadecanoic acid, 20-(eicos-11-enoyloxy)-eicosanoic acid, 21-(eicos-11-enoyloxy)-heneicosanoic acid, 22-(eicos-11-enoyloxy)-docosanoic acid, 23-(eicos-11-enoyloxy)-tricosanoic acid, 24-(eicos-11-enoyloxy)-tetracosanoic acid, 25-(eicos-11-enoyloxy)-pentacosanoic acid, 26-(eicos-11-enoyloxy)-hexacosanoic acid, 27-(eicos-11-enoyloxy)-heptacosanoic acid, 28-(eicos-11-enoyloxy)-octacosanoic acid, 29-(eicos-11-enoyloxy)-nonacosanoic acid, 30-(eicos-11-enoyloxy)-triacontanoic acid, 31-(eicos-11-enoyloxy)-hentriacontanoic acid, 32-(eicos-11-enoyloxy)-dotriacontanoic acid, 33-(eicos-11-enoyloxy)-tritriacontanoic acid, 34-(eicos-11-enoyloxy)-tetratriacontanoic acid, 35-(eicos-11-enoyloxy)-pentatriacontanoic acid, 36-(eicos-11-enoyloxy)-hexatriacontanoic acid, 37-(eicos-11-enoyloxy)-heptatriacontanoic acid, 38-(eicos-11-enoyloxy)-octatriacontanoic acid, 39-(eicos-11-enoyloxy)-nonatriacontanoic acid, and 40-(eicos-11-enoyloxy)-tetracontanoic acid.

In one embodiment, one or more FAHFAs or OAHFAs are selected from the group comprising or consisting of erucic acid based fatty acid esters. In one embodiment, one or more FAHFAs or OAHFAs are selected from the group comprising or consisting of 12-(docos-13-enoyloxy)-dodecanoic acid, 13-(docos-13-enoyloxy)-tridecanoic acid, 14-(docos-13-enoyloxy)-tetradecanoic acid, 15-(docos-13-enoyloxy)-pentadecanoic acid, 16-(docos-13-enoyloxy)-hexadecanoic acid, 17-(docos-13-enoyloxy)-heptadecanoic acid, 18-(docos-13-enoyloxy)-octadecanoic acid, 19-(docos-13-enoyloxy)-nonadecanoic acid, 20-(docos-13-enoyloxy)-eicosanoic acid, 21-(docos-13-enoyloxy)-heneicosanoic acid, 22-(docos-13-enoyloxy)-docosanoic acid, 23-(docos-13-enoyloxy)-tricosanoic acid, 24-(docos-13-enoyloxy)-tetracosanoic acid, 25-(docos-13-enoyloxy)-pentacosanoic acid, 26-(docos-13-enoyloxy)-hexacosanoic acid, 27-(docos-13-enoyloxy)-heptacosanoic acid, 28-(docos-13-enoyloxy)-octacosanoic acid, 29-(docos-13-enoyloxy)-nonacosanoic acid, 30-(docos-13-enoyloxy)-triacontanoic acid, 31-(docos-13-enoyloxy)-hentriacontanoic acid, 32-(docos-13-enoyloxy)-dotriacontanoic acid, 33-(docos-13-enoyloxy)-tritriacontanoic acid, 34-(docos-13-enoyloxy)-tetratriacontanoic acid, 35-(docos-13-enoyloxy)-pentatriacontanoic acid, 36-(docos-13-enoyloxy)-hexatriacontanoic acid, 37-(docos-13-enoyloxy)-heptatriacontanoic acid, 38-(docos-13-enoyloxy)-octatriacontanoic acid, 39-(docos-13-enoyloxy)-nonatriacontanoic acid and 40-(docos-13-enoyloxy)-tetracontanoic acid.

In one embodiment, one or more FAHFAs or OAHFAs are selected from the group comprising or consisting of nervonic acid based fatty acid esters. In one embodiment, one or more FAHFAs or OAHFAs are selected from the group comprising or consisting of 12-(tetracos-15-enoyloxy)-dodecanoic acid, 13-(tetracos-15-enoyloxy)-tridecanoic acid, 14-(tetracos-15-enoyloxy)-tetradecanoic acid, 15-(tetracos-15-enoyloxy)-pentadecanoic acid, 16-(tetracos-15-enoyloxy)-hexadecanoic acid, 17-(tetracos-15-enoyloxy)-heptadecanoic acid, 18-(tetracos-15-enoyloxy)-octadecanoic acid, 19-(tetracos-15-enoyloxy)-nonadecanoic acid, 20-(tetracos-15-enoyloxy)-eicosanoic acid, 21-(tetracos-15-enoyloxy)-heneicosanoic acid, 22-(tetracos-15-enoyloxy)-docosanoic acid, 23-(tetracos-15-enoyloxy)-tricosanoic acid, 24-(tetracos-15-enoyloxy)-tetracosanoic acid, 25-(tetracos-15-enoyloxy)-pentacosanoic acid, 26-(tetracos-15-enoyloxy)-hexacosanoic acid, 27-(tetracos-15-enoyloxy)-heptacosanoic acid, 28-(tetracos-15-enoyloxy)-octacosanoic acid, 29-(tetracos-15-enoyloxy)-nonacosanoic acid, 30-(tetracos-15-enoyloxy)-triacontanoic acid, 31-(tetracos-15-enoyloxy)-hentriacontanoic acid, 32-(tetracos-15-enoyloxy)-dotriacontanoic acid, 33-(tetracos-15-enoyloxy)-tritriacontanoic acid, 34-(tetracos-15-enoyloxy)-tetratriacontanoic acid, 35-(tetracos-15-enoyloxy)-pentatriacontanoic acid, 36-(tetracos-15-enoyloxy)-hexatriacontanoic acid, 37-(tetracos-15-enoyloxy)-heptatriacontanoic acid, 38-(tetracos-15-enoyloxy)-octatriacontanoic acid, 39-(tetracos-15-enoyloxy)-nonatriacontanoic acid, and 40-(tetracos-15-enoyloxy)-tetracontanoic acid.

In one embodiment, one or more FAHFAs or OAHFAs are selected from the group comprising or consisting of linoleic acid based fatty acid esters. In one embodiment, one or more FAHFAs or OAHFAs are selected from the group comprising or consisting of 12-linoleoyloxy-dodecanoic acid, 13-linoleoyloxy-tridecanoic acid, 14-linoleoyloxy-tetradecanoic acid, 15-linoleoyloxy-pentadecanoic acid, 16-linoleoyloxy-hexadecanoic acid, 17-linoleoyloxy-heptadecanoic acid, 18-linoleoyloxy-octadecanoic acid, 19-linoleoyloxy-nonadecanoic acid, 20-linoleoyloxy-eicosanoic acid, 21-linoleoyloxy-heneicosanoic acid, 22-linoleoyloxy-docosanoic acid, 23-linoleoyloxy-tricosanoic acid, 24-linoleoyloxy-tetracosanoic acid, 25-linoleoyloxy-pentacosanoic acid, 26-linoleoyloxy-hexacosanoic acid, 27-linoleoyloxy-heptacosanoic acid, 28-linoleoyloxy-octacosanoic acid, 29-linoleoyloxy-nonacosanoic acid, 30-linoleoyloxy-triacontanoic acid, 31-linoleoyloxy-hentriacontanoic acid, 32-linoleoyloxy-dotriacontanoic acid, 33-linoleoyloxy-tritriacontanoic acid, 34-linoleoyloxy-tetratriacontanoic acid, 35-linoleoyloxy-pentatriacontanoic acid, 36-linoleoyloxy-hexatriacontanoic acid, 37-linoleoyloxy-heptatriacontanoic acid, 38-linoleoyloxy-octatriacontanoic acid, 39-linoleoyloxy-nonatriacontanoic acid, and 40-linoleoyloxy-tetracontanoic acid.

In one embodiment, one or more FAHFAs or OAHFAs are selected from the group comprising or consisting of linolenic acid based fatty acid esters. In one embodiment, one or more FAHFAs or OAHFAs are selected from the group comprising or consisting of 12-(((9Z,12Z,15Z)-octadeca-9,12,15-trienoyl)oxy)-dodecanoic acid, 13-(((9Z,12Z,15Z)-octadeca-9,12,15-trienoyl)oxy)-tridecanoic acid, 14-(((9Z,12Z,15Z)-octadeca-9,12,15-trienoyl)oxy)-tetradecanoic acid, 15-(((9Z,12Z,15Z)-octadeca-9,12,15-trienoyl)oxy)-pentadecanoic acid, 16-(((9Z,12Z,15Z)-octadeca-9,12,15-trienoyl)oxy)-hexadecanoic acid, 17-(((9Z,12Z,15Z)-octadeca-9,12,15-trienoyl)oxy)-heptadecanoic acid, 18-(((9Z,12Z,15Z)-octadeca-9,12,15-trienoyl)oxy)-octadecanoic acid, 19-(((9Z,12Z,15Z)-octadeca-9,12,15-trienoyl)oxy)-nonadecanoic acid, 20-(((9Z,12Z,15Z)-octadeca-9,12,15-trienoyl)oxy)-eicosanoic acid, 21-(((9Z,12Z,15Z)-octadeca-9,12,15-trienoyl)oxy)-heneicosanoic acid, 22-(((9Z,12Z,15Z)-octadeca-9,12,15-trienoyl)oxy)-docosanoic acid, 23-(((9Z,12Z,15Z)-octadeca-9,12,15-trienoyl)oxy)-tricosanoic acid, 24-(((9Z,12Z,15Z)-octadeca-9,12,15-trienoyl)oxy)-tetracosanoic acid, 25-(((9Z,12Z,15Z)-octadeca-9,12,15-trienoyl)oxy)-pentacosanoic acid, 26-(((9Z,12Z,15Z)-octadeca-9,12,15-trienoyl)oxy)-hexacosanoic acid, 27-(((9Z,12Z,15Z)-octadeca-9,12,15-trienoyl)oxy)-heptacosanoic acid, 28-(((9Z,12Z,15Z)-octadeca-9,12,15-trienoyl)oxy)-octacosanoic acid, 29-(((9Z,12Z,15Z)-octadeca-9,12,15-trienoyl)oxy)-nonacosanoic acid, 30-(((9Z,12Z,15Z)-octadeca-9,12,15-trienoyl)oxy)-triacontanoic acid, 31-(((9Z,12Z,15Z)-octadeca-9,12,15-trienoyl)oxy)-hentriacontanoic acid, 32-(((9Z,12Z,15Z)-octadeca-9,12,15-trienoyl)oxy)-dotriacontanoic acid, 33-(((9Z,12Z,15Z)-octadeca-9,12,15-trienoyl)oxy)-tritriacontanoic acid, 34-(((9Z,12Z,15Z)-octadeca-9,12,15-trienoyl)oxy)-tetratriacontanoic acid, 35-(((9Z,12Z,15Z)-octadeca-9,12,15-trienoyl)oxy)-pentatriacontanoic acid, 36-(((9Z,12Z,15Z)-octadeca-9,12,15-trienoyl)oxy)-hexatriacontanoic acid, 37-(((9Z,12Z,15Z)-octadeca-9,12,15-trienoyl)oxy)-heptatriacontanoic acid, 38-(((9Z,12Z,15Z)-octadeca-9,12,15-trienoyl)oxy)-octatriacontanoic acid, 39-(((9Z,12Z,15Z)-octadeca-9,12,15-trienoyl)oxy)-nonatriacontanoic acid, and 40-(((9Z,12Z,15Z)-octadeca-9,12,15-trienoyl)oxy)-tetracontanoic acid.

In one embodiment, one or more structural analogues of FAHFA or OAHFA are selected from the group comprising or consisting of oleic acid-based alcohols. In one embodiment, one or more structural analogues of FAHFA or OAHFA are selected from the group comprising or consisting of 12-hydroxydodecyl oleate, 13-hydroxytridecyl oleate, 14-hydroxytetradecyl oleate, 15-hydroxypentadecyl oleate, 16-hydroxyhexadecyl oleate, 17-hydroxyheptadecyl oleate, 18-hydroxyoctadecyl oleate, 19-hydroxynonadecyl oleate, 20-hydroxyeicosyl oleate, 21-hydroxyheneicosyl oleate, 22-hydroxydocosyl oleate, 23-hydroxytricosyl oleate, 24-hydroxytetracosyl oleate, 25-hydroxypentacosyl oleate, 26-hydroxyhexacosyl oleate, 27-hydroxyheptacosyl oleate, 28-hydroxyoctacosyl oleate, 29-hydroxynonacosyl oleate, 30-hydroxytriacontyl oleate, 31-hydroxyhentriacontyl oleate, 32-hydroxydotriacontyl oleate, 33-hydroxytritriacontyl oleate, 34-hydroxytetratriacontyl oleate, 35-hydroxypentatriacontyl oleate, 36-hydroxyhexatriacontyl oleate, 37-hydroxyheptatriacontyl oleate, 38-hydroxyoctatriacontyl oleate, 39-hydroxynonatriacontyl oleate and 40-hydroxytetracontyl oleate.

In one embodiment, one or more structural analogues of FAHFA or OAHFA are selected from the group comprising or consisting of palmitoleic acid-based alcohols. In one embodiment, one or more structural analogues of FAHFA or OAHFA are selected from the group comprising or consisting of 12-hydroxydodecyl palmitoleate, 13-hydroxytridecyl palmitoleate, 14-hydroxytetradecyl palmitoleate, 15-hydroxypentadecyl palmitoleate, 16-hydroxyhexadecyl palmitoleate, 17-hydroxyheptadecyl palmitoleate, 18-hydroxyoctadecyl palmitoleate, 19-hydroxynonadecyl palmitoleate, 20-hydroxyeicosyl palmitoleate, 21-hydroxyheneicosyl palmitoleate, 22-hydroxydocosyl palmitoleate, 23-hydroxytricosyl palmitoleate, 24-hydroxytetracosyl palmitoleate, 25-hydroxypentacosyl palmitoleate, 26-hydroxyhexacosyl palmitoleate, 27-hydroxyheptacosyl palmitoleate, 28-hydroxyoctacosyl palmitoleate, 29-hydroxynonacosyl palmitoleate, 30-hydroxytriacontyl palmitoleate, 31-hydroxyhentriacontyl palmitoleate, 32-hydroxydotriacontyl palmitoleate, 33-hydroxytritriacontyl palmitoleate, 34-hydroxytetratriacontyl palmitoleate, 35-hydroxypentatriacontyl palmitoleate, 36-hydroxyhexatriacontyl palmitoleate, 37-hydroxyheptatriacontyl palmitoleate, 38-hydroxyoctatriacontyl palmitoleate, 39-hydroxynonatriacontyl palmitoleate and 40-hydroxytetracontyl palmitoleate.

In one embodiment, one or more structural analogues of FAHFA or OAHFA are selected from the group comprising or consisting of myristoleic acid-based alcohols. In one embodiment, one or more structural analogues of FAHFA or OAHFA are selected from the group comprising or consisting of 12-hydroxydodecyl myristoleate, 13-hydroxytridecyl myristoleate, 14-hydroxytetradecyl myristoleate, 15-hydroxypentadecyl myristoleate, 16-hydroxyhexadecyl myristoleate, 17-hydroxyheptadecyl myristoleate, 18-hydroxyoctadecyl myristoleate, 19-hydroxynonadecyl myristoleate, 20-hydroxyeicosyl myristoleate, 21-hydroxyheneicosyl myristoleate, 22-hydroxydocosyl myristoleate, 23-hydroxytricosyl myristoleate, 24-hydroxytetracosyl myristoleate, 25-hydroxypentacosyl myristoleate, 26-hydroxyhexacosyl myristoleate, 27-hydroxyheptacosyl myristoleate, 28-hydroxyoctacosyl myristoleate, 29-hydroxynonacosyl myristoleate, 30-hydroxytriacontyl myristoleate, 31-hydroxyhentriacontyl myristoleate, 32-hydroxydotriacontyl myristoleate, 33-hydroxytritriacontyl myristoleate, 34-hydroxytetratriacontyl myristoleate, 35-hydroxypentatriacontyl myristoleate, 36-hydroxyhexatriacontyl myristoleate, 37-hydroxyheptatriacontyl myristoleate, 38-hydroxyoctatriacontyl myristoleate, 39-hydroxynonatriacontyl myristoleate and 40-hydroxytetracontyl myristoleate.

In one embodiment, one or more structural analogues of FAHFA or OAHFA are selected from the group comprising or consisting of lauric acid-based alcohols. In one embodiment, one or more structural analogues of FAHFA or OAHFA are selected from the group comprising or consisting of 12-hydroxydodecyl laurate, 13-hydroxytridecyl laurate, 14-hydroxytetradecyl laurate, 15-hydroxypentadecyl laurate, 16-hydroxyhexadecyl laurate, 17-hydroxyheptadecyl laurate, 18-hydroxyoctadecyl laurate, 19-hydroxynonadecyl laurate, 20-hydroxyeicosyl laurate, 21-hydroxyheneicosyl laurate, 22-hydroxydocosyl laurate, 23-hydroxytricosyl laurate, 24-hydroxytetracosyl laurate, 25-hydroxypentacosyl laurate, 26-hydroxyhexacosyl laurate, 27-hydroxyheptacosyl laurate, 28-hydroxyoctacosyl laurate, 29-hydroxynonacosyl laurate, 30-hydroxytriacontyl laurate, 31-hydroxyhentriacontyl laurate, 32-hydroxydotriacontyl laurate, 33-hydroxytritriacontyl laurate, 34-hydroxytetratriacontyl laurate, 35-hydroxypentatriacontyl laurate, 36-hydroxyhexatriacontyl laurate, 37-hydroxyheptatriacontyl laurate, 38-hydroxyoctatriacontyl laurate, 39-hydroxynonatriacontyl laurate and 40-hydroxytetracontyl laurate.

In one embodiment, one or more structural analogues of FAHFA or OAHFA are selected from the group comprising or consisting of paullinic acid-based alcohols. In one embodiment, one or more structural analogues of FAHFA or OAHFA are selected from the group comprising or consisting of 12-hydroxydodecyl eicos-13-enoate, 13-hydroxytridecyl eicos-13-enoate, 14-hydroxytetradecyl eicos-13-enoate, 15-hydroxypentadecyl eicos-13-enoate, 16-hydroxyhexadecyl eicos-13-enoate, 17-hydroxyheptadecyl eicos-13-enoate, 18-hydroxyoctadecyl eicos-13-enoate, 19-hydroxynonadecyl eicos-13-enoate, 20-hydroxyeicosyl eicos-13-enoate, 21-hydroxyheneicosyl eicos-13-enoate, 22-hydroxydocosyl eicos-13-enoate, 23-hydroxytricosyl eicos-13-enoate, 24-hydroxytetracosyl eicos-13-enoate, 25-hydroxypentacosyl eicos-13-enoate, 26-hydroxyhexacosyl eicos-13-enoate, 27-hydroxyheptacosyl eicos-13-enoate, 28-hydroxyoctacosyl eicos-13-enoate, 29-hydroxynonacosyl eicos-13-enoate, 30-hydroxytriacontyl eicos-13-enoate, 31-hydroxyhentriacontyl eicos-13-enoate, 32-hydroxydotriacontyl eicos-13-enoate, 33-hydroxytritriacontyl eicos-13-enoate, 34-hydroxytetratriacontyl eicos-13-enoate, 35-hydroxypentatriacontyl eicos-13-enoate, 36-hydroxyhexatriacontyl eicos-13-enoate, 37-hydroxyheptatriacontyl eicos-13-enoate, 38-hydroxyoctatriacontyl eicos-13-enoate, 39-hydroxynonatriacontyl eicos-13-enoate and 40-hydroxytetracontyl eicos-13-enoate.

In one embodiment, one or more structural analogues of FAHFA or OAHFA are selected from the group comprising or consisting of gondoic acid-based alcohols. In one embodiment, one or more structural analogues of FAHFA or OAHFA are selected from the group comprising or consisting of 12-hydroxydodecyl eicos-11-enoate, 13-hydroxytridecyl eicos-11-enoate, 14-hydroxytetradecyl eicos-11-enoate, 15-hydroxypentadecyl eicos-11-enoate, 16-hydroxyhexadecyl eicos-11-enoate, 17-hydroxyheptadecyl eicos-11-enoate, 18-hydroxyoctadecyl eicos-11-enoate, 19-hydroxynonadecyl eicos-11-enoate, 20-hydroxyeicosyl eicos-11-enoate, 21-hydroxyheneicosyl eicos-11-enoate, 22-hydroxydocosyl eicos-11-enoate, 23-hydroxytricosyl eicos-11-enoate, 24-hydroxytetracosyl eicos-11-enoate, 25-hydroxypentacosyl eicos-11-enoate, 26-hydroxyhexacosyl eicos-11-enoate, 27-hydroxyheptacosyl eicos-11-enoate, 28-hydroxyoctacosyl eicos-11-enoate, 29-hydroxynonacosyl eicos-11-enoate, 30-hydroxytriacontyl eicos-11-enoate, 31-hydroxyhentriacontyl eicos-11-enoate, 32-hydroxydotriacontyl eicos-11-enoate, 33-hydroxytritriacontyl eicos-11-enoate, 34-hydroxytetratriacontyl eicos-11-enoate, 35-hydroxypentatriacontyl eicos-11-enoate, 36-hydroxyhexatriacontyl eicos-11-enoate, 37-hydroxyheptatriacontyl eicos-11-enoate, 38-hydroxyoctatriacontyl eicos-11-enoate, 39-hydroxynonatriacontyl eicos-11-enoate and 40-hydroxytetracontyl eicos-11-enoate.

In one embodiment, one or more structural analogues of FAHFA or OAHFA are selected from the group comprising or consisting of erucic acid-based alcohols, In one embodiment, one or more structural analogues of FAHFA or OAHFA are selected from the group comprising or consisting of 12-hydroxydodecyl docos-13-enoate, 13-hydroxytridecyl docos-13-enoate, 14-hydroxytetradecyl docos-13-enoate, 15-hydroxypentadecyl docos-13-enoate, 16-hydroxyhexadecyl docos-13-enoate, 17-hydroxyheptadecyl docos-13-enoate, 18-hydroxyoctadecyl docos-13-enoate, 19-hydroxynonadecyl docos-13-enoate, 20-hydroxyeicosyl docos-13-enoate, 21-hydroxyheneicosyl docos-13-enoate, 22-hydroxydocosyl docos-13-enoate, 23-hydroxytricosyl docos-13-enoate, 24-hydroxytetracosyl docos-13-enoate, 25-hydroxypentacosyl docos-13-enoate, 26-hydroxyhexacosyl docos-13-enoate, 27-hydroxyheptacosyl docos-13-enoate, 28-hydroxyoctacosyl docos-13-enoate, 29-hydroxynonacosyl docos-13-enoate, 30-hydroxytriacontyl docos-13-enoate, 31-hydroxyhentriacontyl docos-13-enoate, 32-hydroxydotriacontyl docos-13-enoate, 33-hydroxytritriacontyl docos-13-enoate, 34-hydroxytetratriacontyl docos-13-enoate, 35-hydroxypentatriacontyl docos-13-enoate, 36-hydroxyhexatriacontyl docos-13-enoate, 37-hydroxyheptatriacontyl docos-13-enoate, 38-hydroxyoctatriacontyl docos-13-enoate, 39-hydroxynonatriacontyl docos-13-enoate, and 40-hydroxytetracontyl docos-13-enoate.

In one embodiment, one or more structural analogues of FAHFA or OAHFA are selected from the group comprising or consisting of nervonic acid-based alcohols, In one embodiment, one or more structural analogues of FAHFA or OAHFA are selected from the group comprising or consisting of 12-hydroxydodecyl tetracos-15-enoate, 13-hydroxytridecyl tetracos-15-enoate, 14-hydroxytetradecyl tetracos-15-enoate, 15-hydroxypentadecyl tetracos-15-enoate, 16-hydroxyhexadecyl tetracos-15-enoate, 17-hydroxyheptadecyl tetracos-15-enoate, 18-hydroxyoctadecyl tetracos-15-enoate, 19-hydroxynonadecyl tetracos-15-enoate, 20-hydroxyeicosyl tetracos-15-enoate, 21-hydroxyheneicosyl tetracos-15-enoate, 22-hydroxydocosyl tetracos-15-enoate, 23-hydroxytricosyl tetracos-15-enoate, 24-hydroxytetracosyl tetracos-15-enoate, 25-hydroxypentacosyl tetracos-15-enoate, 26-hydroxyhexacosyl tetracos-15-enoate, 27-hydroxyheptacosyl tetracos-15-enoate, 28-hydroxyoctacosyl tetracos-15-enoate, 29-hydroxynonacosyl tetracos-15-enoate, 30-hydroxytriacontyl tetracos-15-enoate, 31-hydroxyhentriacontyl tetracos-15-enoate, 32-hydroxydotriacontyl tetracos-15-enoate, 33-hydroxytritriacontyl tetracos-15-enoate, 34-hydroxytetratriacontyl tetracos-15-enoate, 35-hydroxypentatriacontyl tetracos-15-enoate, 36-hydroxyhexatriacontyl tetracos-15-enoate, 37-hydroxyheptatriacontyl tetracos-15-enoate, 38-hydroxyoctatriacontyl tetracos-15-enoate, 39-hydroxynonatriacontyl tetracos-15-enoate, and 40-hydroxytetracontyl tetracos-15-enoate.

In one embodiment, one or more structural analogues of FAHFA or OAHFA are selected from the group comprising or consisting of linoleic acid-based alcohols. In one embodiment, one or more structural analogues of FAHFA or OAHFA are selected from the group comprising or consisting of 12-hydroxydodecyl linoleate, 13-hydroxytridecyl linoleate, 14-hydroxytetradecyl linoleate, 15-hydroxypentadecyl linoleate, 16-hydroxyhexadecyl linoleate, 17-hydroxyheptadecyl linoleate, 18-hydroxyoctadecyl linoleate, 19-hydroxynonadecyl linoleate, 20-hydroxyeicosyl linoleate, 21-hydroxyheneicosyl linoleate, 22-hydroxydocosyl linoleate, 23-hydroxytricosyl linoleate, 24-hydroxytetracosyl linoleate, 25-hydroxypentacosyl linoleate, 26-hydroxyhexacosyl linoleate, 27-hydroxyheptacosyl linoleate, 28-hydroxyoctacosyl linoleate, 29-hydroxynonacosyl linoleate, 30-hydroxytriacontyl linoleate, 31-hydroxyhentriacontyl linoleate, 32-hydroxydotriacontyl linoleate, 33-hydroxytritriacontyl linoleate, 34-hydroxytetratriacontyl linoleate, 35-hydroxypentatriacontyl linoleate, 36-hydroxyhexatriacontyl linoleate, 37-hydroxyheptatriacontyl linoleate, 38-hydroxyoctatriacontyl linoleate, 39-hydroxynonatriacontyl linoleate, and 40-hydroxytetracontyl linoleate.

In one embodiment, one or more structural analogues of FAHFA or OAHFA are selected from the group comprising or consisting of linolenic acid-based alcohols. 12-hydroxydodecyl linolenate, 13-hydroxytridecyl linolenate, 14-hydroxytetradecyl linolenate, 15-hydroxypentadecyl linolenate, 16-hydroxyhexadecyl linolenate, 17-hydroxyheptadecyl linolenate, 18-hydroxyoctadecyl linolenate, 19-hydroxynonadecyl linolenate, 20-hydroxyeicosyl linolenate, 21-hydroxyheneicosyl linolenate, 22-hydroxydocosyl linolenate, 23-hydroxytricosyl linolenate, 24-hydroxytetracosyl linolenate, 25-hydroxypentacosyl linolenate, 26-hydroxyhexacosyl linolenate, 27-hydroxyheptacosyl linolenate, 28-hydroxyoctacosyl linolenate, 29-hydroxynonacosyl linolenate, 30-hydroxytriacontyl linolenate, 31-hydroxyhentriacontyl linolenate, 32-hydroxydotriacontyl linolenate, 33-hydroxytritriacontyl linolenate, 34-hydroxytetratriacontyl linolenate, 35-hydroxypentatriacontyl linolenate, 36-hydroxyhexatriacontyl linolenate, 37-hydroxyheptatriacontyl linolenate, 38-hydroxyoctatriacontyl linolenate, 39-hydroxynonatriacontyl linolenate, and 40-hydroxytetracontyl linolenate.

In one embodiment, the FAHFA is selected from 18-(oleoyloxy)stearic acid (18:0/18:1-OAHFA; 18-OAHFA), 12-(linoleoyloxy)dodecanoic acid, 20-(linoleoyloxy)eicosanoic acid, 12-(palmitoleoyloxy)dodecanoic acid, 20-(palmitoleoyloxy)eicosanoic acid, 12-(palmitoyloxy)dodecanoic acid, 20-(palmitoyloxy)eicosanoic acid, 12-(stearoyloxy)dodecanoic acid, 20-(stearoyloxy)eicosanoic acid, 12-OAHFA (12-(oleoyloxy)dodecanoic acid), 15-OAHFA (15-(oleoyloxy)pentadecanoic acid), 20-OAHFA (20-(oleoyloxy)eicosanoic acid), 22-OAHFA (22-(oleoyloxy)docosanoic acid), 20:1-OAHFA ((12Z)-20-(oleoyloxy)eicos-12-enoic acid) and 29:1-OAHFA ((21Z)-29-(oleoyloxy)nonacos-21-enoic acid).

In one embodiment, the FAHFA is selected from 18-(oleoyloxy)stearic acid (18:0/18:1-OAHFA; 18-OAHFA), 12-OAHFA (12-(oleoyloxy)dodecanoic acid), 15-OAHFA (15-(oleoyxy)pentadecanoic acid), 20-OAHFA (20-(oleoyloxy)eicosanoic acid), 22-OAHFA (22-(oleoyloxy)docosanoic acid), 20:1-OAHFA ((12Z)-20-(oleoyloxy)eicos-12-enoic acid) and 29:1-OAHFA ((21Z)-29-(oleoyloxy)nonacos-21-enoic acid).

In one embodiment, the one or more FAHFAs are selected from 12-OAHFA (12-(oleoyloxy)dodecanoic acid), 15-OAHFA (15-(oleoyxy)pentadecanoic acid), 20-OAHFA (20-(oleoyloxy)eicosanoic acid), 22-OAHFA (22-(oleoyloxy)docosanoic acid), 20:1-OAHFA ((12Z)-20-(oleoyloxy)eicos-12-enoic acid) and 29:1-OAHFA ((21Z)-29-(oleoyloxy)nonacos-21-enoic acid).

In one embodiment, the one or more FAHFAs are selected from 18-(oleoyloxy)stearic acid (18:0/18:1-OAHFA; 18-OAHFA), 12-(linoleoyloxy)dodecanoic acid, 20-(linoleoyloxy)eicosanoic acid, 12-(palmitoleoyloxy)dodecanoic acid, 20-(palmitoleoyloxy)eicosanoic acid, 12-(palmitoyloxy)dodecanoic acid, 20-(palmitoyloxy)eicosanoic acid, 12-(stearoyloxy)dodecanoic acid, 20-(stearoyloxy)eicosanoic acid.

In further embodiments, the FAHFA is selected from the group consisting of 20-(palmitoleoyloxy)eicosanoic acid, 20-(oleoyloxy)eicosanoic acid and 18-(oleoyloxy)stearic acid (18:0/18:1-OAHFA; 18-OAHFA).

In particular embodiments, the FAHFA is selected from the group consisting of 20-(oleoyloxy)eicosanoic acid and 18-(oleoyloxy)stearic acid (18:0/18:1-OAHFA; 18-OAHFA).

In one embodiment, the FAHFA is 20-(oleoyloxy)eicosanoic acid.

In a further embodiment, the FAHFA is 18-(oleoyloxy)stearic acid.

In a further embodiment, the FAHFA is 20-(palmitoleoyloxy)eicosanoic acid.

The carbon chain length of one or more wax esters or analogues thereof suitable for the composition of the present invention can vary. In one embodiment there is no maximum carbon chain length. In one embodiment the carbon chain length of one or more wax esters or structural analogs thereof is C15-C100, C19-C72, C20-C55, C20-C50, C20-C40, C20-C35, C20-C25, C25-C45, C25-C40, C25-C35 or C25-C30. In one embodiment the carbon chain length of one or more wax esters or analogues thereof is C20, C21, C22, C23, C24, C25, C26, C27, C28, C29, C30, C31, C32, C33, C34, C35, C36, C37, C38, C39, C40, C41, C42, C43, C44, C45, C46, C47, C48, C49, C50, C51, C52, C53, C54 or C55.

In one embodiment the wax ester of a structural analogue thereof has the following formula (II):

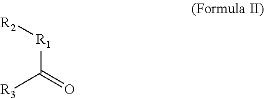

wherein
R₁ is a carbon atom, an oxygen atom or a nitrogen atom;
R₂ is a linear or branched C9-C50 alkyl, alkenyl or alkynyl chain, or a structural analogue thereof;
R₃ is a linear or branched C9-C50 alkyl, alkenyl or alkynyl chain, or a structural analogue thereof.

In one embodiment, the wax ester is a linear wax ester. In one embodiment, the wax ester is a branched wax ester.

In one embodiment the acyl chain of one or more wax esters are selected from the group comprising or consisting of the following fatty acids: oleic acid, palmitoleic acid, myristoleic acid, lauric acid, paullinic acid, gondoic acid, erucic acid, nervonic acid, linoleic acid, and linolenic acid, and the alkoxy chain of one or more wax esters are selected from the group comprising or consisting of a straight-chain fatty alcohol, iso-branched fatty alcohol, and anteiso-branched fatty alcohol; and/or a structural analogue thereof.

In one embodiment one or more wax esters are selected from the group comprising or consisting of oleic acid based esters. In one embodiment one or more wax esters are selected from the group comprising or consisting of lauryl oleate, tridecyl oleate, myristyl oleate, pentadecyl oleate, palmityl oleate, heptadecyl oleate, stearyl oleate, nonadecyl oleate, arachidyl oleate (AO), heneicosyl oleate, behenyl oleate (BO), tricosyl oleate, lignoceryl oleate, pentacosyl oleate, hexacosyl oleate, heptacosyl oleate, octacosyl oleate, nonacosyl oleate, triacontyl oleate, hentriacontyl oleate, dotriacontyl oleate, tritriacontyl oleate, tetratriacontyl oleate, pentatriacontyl oleate, hexatriacontyl oleate, heptatriacontyl oleate, octatriacontyl oleate, nonatriacontyl oleate and tetracontyl oleate.

In one embodiment one or more wax esters are selected from the group comprising or consisting of iso-branched alkyl oleates. In one embodiment one or more wax esters are selected from the group comprising or consisting of 11-methyllauryl oleate, 12-methyltridecyl oleate, 13-methylmyristyl oleate, 14-methylpentadecyl oleate, 15-methylpalmityl oleate, 16-methylheptadecyl oleate, 17-methylstearyl oleate, 18-methylnonadecyl oleate, 19-methylarachidyl oleate, 20-methylheneicosyl oleate, 21-methylbehenyl oleate, 22-methyltricosyl oleate, 23-methyllignoceryl oleate, 24-methylpentacosyl oleate, 25-methylhexacosyl oleate, 26-methylheptacosyl oleate, 27-methyloctacosyl oleate, 28-methylnonacosyl oleate, 29-methyltriacontyl oleate, 30-methylhentriacontyl oleate, 31-methyldotriacontyl oleate, 32-methyltritriacontyl oleate, 33-methyltetratriacontyl oleate, 34-methylpentatriacontyl oleate, 35-methylhexatriacontyl oleate, 36-methylheptatriacontyl oleate, 37-methyloctatriacontyl oleate, 38-methylnonatriacontyl oleate, 39-methyltetracontyl oleate.

In one embodiment one or more wax esters are selected from the group comprising or consisting of anteiso-branched alkyl oleates. In one embodiment one or more wax esters are selected from the group comprising or consisting of 10-methyllauryl oleate, 11-methyltridecyl oleate, 12-methylmyristyl oleate, 13-methylpentadecyl oleate, 14-methylpalmityl oleate, 15-methylheptadecyl oleate, 16-methylstearyl oleate, 17-methylnonadecyl oleate, 18-methylarachidyl oleate, 19-methylheneicosyl oleate, 20-methylbehenyl oleate, 21-methyltricosyl oleate, 22-methyllignoceryl oleate, 23-methylpentacosyl oleate, 24-methylhexacosyl oleate, 25-methylheptacosyl oleate, 26-methyloctacosyl oleate, 27-methylnonacosyl oleate, 28-methyltriacontyl oleate, 29-methylhentriacontyl oleate, 30-methyldotriacontyl oleate, 31-methyltritriacontyl oleate, 32-methyltetratriacontyl oleate, 33-methylpentatriacontyl oleate, 34-methylhexatriacontyl oleate, 35-methylheptatriacontyl oleate, 36-methyloctatriacontyl oleate, 37-methylnonatriacontyl oleate, and 38-methyltetracontyl oleate.

In one embodiment one or more wax esters are selected from the group comprising or consisting of palmitoleic acid-based esters. In one embodiment one or more wax esters are selected from the group comprising or consisting of lauryl palmitoleate, tridecyl palmitoleate, myristyl palmitoleate, pentadecyl palmitoleate, palmityl palmitoleate, heptadecyl palmitoleate, stearyl palmitoleate, nonadecyl palmitoleate, arachidyl palmitoleate, heneicosyl palmitoleate, behenyl palmitoleate, tricosyl palmitoleate, lignoceryl palmitoleate, pentacosyl palmitoleate, hexacosyl palmitoleate, heptacosyl palmitoleate, octacosyl palmitoleate, nonacosyl palmitoleate, triacontyl palmitoleate, hentriacontyl palmitoleate, dotriacontyl palmitoleate, tritriacontyl palmitoleate, tetratriacontyl palmitoleate, pentatriacontyl palmitoleate, hexatriacontyl palmitoleate, heptatriacontyl palmitoleate, octatriacontyl palmitoleate, nonatriacontyl palmitoleate, tetracontyl palmitoleate.

In one embodiment one or more wax esters are selected from the group comprising or consisting of iso-branched alkyl palmitoleates. In one embodiment one or more wax esters are selected from the group comprising or consisting of 11-methyllauryl palmitoleate, 12-methyltridecyl palmitoleate, 13-methylmyristyl palmitoleate, 14-methylpentadecyl palmitoleate, 15-methylpalmityl palmitoleate, 16-methylheptadecyl palmitoleate, 17-methylstearyl palmitoleate, 18-methylnonadecyl palmitoleate, 19-methylarachidyl palmitoleate, 20-methylheneicosyl palmitoleate, 21-methylbehenyl palmitoleate, 22-methyltricosyl palmitoleate, 23-methyllignoceryl palmitoleate, 24-methylpentacosyl palmitoleate, 25-methylhexacosyl palmitoleate, 26-methylheptacosyl palmitoleate, 27-methyloctacosyl palmitoleate, 28-methylnonacosyl palmitoleate, 29-methyltriacontyl palmitoleate, 30-methylhentriacontyl palmitoleate, 31-methyldotriacontyl palmitoleate, 32-methyltritriacontyl palmitoleate, 33-methyltetratriacontyl palmitoleate, 34-methylpentatriacontyl palmitoleate, 35-methylhexatriacontyl palmitoleate, 36-methylheptatriacontyl palmitoleate, 37-methyloctatriacontyl palmitoleate, 38-methylnonatriacontyl palmitoleate, and 39-methyltetracontyl palmitoleate.

In one embodiment one or more wax esters are selected from the group comprising or consisting of anteiso-branched alkyl palmitoleates. In one embodiment one or more wax esters are selected from the group comprising or consisting of 10-methyllauryl palmitoleate, 11-methyltridecyl palmitoleate, 12-methylmyristyl palmitoleate, 13-methylpentadecyl palmitoleate, 14-methylpalmityl palmitoleate, 15-methylheptadecyl palmitoleate, 16-methylstearyl palmitoleate, 17-methylnonadecyl palmitoleate, 18-methylarachidyl palmitoleate, 19-methylheneicosyl palmitoleate, 20-methylbehenyl palmitoleate, 21-methyltricosyl palmitoleate, 22-methyllignoceryl palmitoleate, 23-methylpentacosyl palmitoleate, 24-methylhexacosyl palmitoleate, 25-methylheptacosyl palmitoleate, 26-methyloctacosyl palmitoleate, 27-methylnonacosyl palmitoleate, 28-methyltriacontyl palmitoleate, 29-methylhentriacontyl palmitoleate, 30-methyldotriacontyl palmitoleate, 31-methyltritriacontyl palmitoleate, 32-methyltetratriacontyl palmitoleate, 33-methylpentatriacontyl palmitoleate, 34-methylhexatriacontyl palmitoleate, 35-methylheptatriacontyl palmitoleate, 36-methyloctatriacontyl palmitoleate, 37-methylnonatriacontyl palmitoleate, and 38-methyltetracontyl palmitoleate.

In one embodiment one or more wax esters are selected from the group comprising or consisting of myristoleic acid based esters. In one embodiment one or more wax esters are selected from the group comprising or consisting of lauryl myristoleate, tridecyl myristoleate, myristyl myristoleate, pentadecyl myristoleate, palmityl myristoleate, heptadecyl myristoleate, stearyl myristoleate, nonadecyl myristoleate, arachidyl myristoleate, heneicosyl myristoleate, behenyl myristoleate, tricosyl myristoleate, lignoceryl myristoleate, pentacosyl myristoleate, hexacosyl myristoleate, heptacosyl myristoleate, octacosyl myristoleate, nonacosyl myristoleate, triacontyl myristoleate, hentriacontyl myristoleate, dotriacontyl myristoleate, tritriacontyl myristoleate, tetratriacontyl myristoleate, pentatriacontyl myristoleate, hexatriacontyl myristoleate, heptatriacontyl myristoleate, octatriacontyl myristoleate, nonatriacontyl myristoleate, tetracontyl myristoleate, 11-methyllauryl myristoleate, 12-methyltridecyl myristoleate, 13-methylmyristyl myristoleate, 14-methylpentadecyl myristoleate, 15-methylpalmityl myristoleate, 16-methylheptadecyl myristoleate, and 17-methylstearyl myristoleate.

In one embodiment one or more wax esters are selected from the group comprising or consisting of iso-branched alkyl myristoleates. In one embodiment one or more wax esters are selected from the group comprising or consisting of 18-methylnonadecyl myristoleate, 19-methylarachidyl myristoleate, 20-methylheneicosyl myristoleate, 21-methylbehenyl myristoleate, 22-methyltricosyl myristoleate, 23-methyllignoceryl myristoleate, 24-methylpentacosyl myristoleate, 25-methylhexacosyl myristoleate, 26-methylheptacosyl myristoleate, 27-methyloctacosyl myristoleate, 28-methylnonacosyl myristoleate, 29-methyltriacontyl myristoleate, 30-methylhentriacontyl myristoleate, 31-methyldotriacontyl myristoleate, 32-methyltritriacontyl myristoleate, 33-methyltetratriacontyl myristoleate, 34-methylpentatriacontyl myristoleate, 35-methylhexatriacontyl myristoleate, 36-methylheptatriacontyl myristoleate, 37-methyloctatriacontyl myristoleate, 38-methylnonatriacontyl myristoleate, and 39-methyltetracontyl myristoleate.

In one embodiment one or more wax esters are selected from the group comprising or consisting of anteiso-branched alkyl myristoleates. In one embodiment one or more wax esters are selected from the group comprising or consisting of 10-methyllauryl myristoleate, 11-methyltridecyl myristoleate, 12-methylmyristyl myristoleate, 13-methylpentadecyl myristoleate, 14-methylpalmityl myristoleate, 15-methylheptadecyl myristoleate, 16-methylstearyl myristoleate, 17-methylnonadecyl myristoleate, 18-methylarachidyl myristoleate, 19-methylheneicosyl myristoleate, 20-methylbehenyl myristoleate, 21-methyltricosyl myristoleate, 22-methyllignoceryl myristoleate, 23-methylpentacosyl myristoleate, 24-methylhexacosyl myristoleate, 25-methylheptacosyl myristoleate, 26-methyloctacosyl myristoleate, 27-methylnonacosyl myristoleate, 28-methyltriacontyl myristoleate, 29-methylhentriacontyl myristoleate, 30-methyldotriacontyl myristoleate, 31-methyltritriacontyl myristoleate, 32-methyltetratriacontyl myristoleate, 33-methylpentatriacontyl myristoleate, 34-methylhexatriacontyl myristoleate, 35-methylheptatriacontyl myristoleate, 36-methyloctatriacontyl myristoleate, 37-methylnonatriacontyl myristoleate, and 38-methyltetracontyl myristoleate.

In one embodiment one or more wax esters are selected from the group comprising or consisting of lauric acid based esters. In one embodiment one or more wax esters are selected from the group comprising or consisting of lauryl laurate, tridecyl laurate, myristyl laurate, pentadecyl laurate, palmityl laurate, heptadecyl laurate, stearyl laurate, nonadecyl laurate, arachidyl laurate, heneicosyl laurate, behenyl laurate, tricosyl laurate, lignoceryl laurate, pentacosyl laurate, hexacosyl laurate, heptacosyl laurate, octacosyl laurate, nonacosyl laurate, triacontyl laurate, hentriacontyl laurate, dotriacontyl laurate, tritriacontyl laurate, tetratriacontyl laurate, pentatriacontyl laurate, hexatriacontyl laurate, heptatriacontyl laurate, octatriacontyl laurate, nonatriacontyl laurate and tetracontyl laurate.

In one embodiment one or more wax esters are selected from the group comprising or consisting of iso-branched alkyl laurates. In one embodiment one or more wax esters are selected from the group comprising or consisting of 11-methyllauryl laurate, 12-methyltridecyl laurate, 13-methylmyristyl laurate, 14-methylpentadecyl laurate, 15-methylpalmityl laurate, 16-methylheptadecyl laurate, 17-methylstearyl laurate, 18-methylnonadecyl laurate, 19-methylarachidyl laurate, 20-methylheneicosyl laurate, 21-methylbehenyl laurate, 22-methyltricosyl laurate, 23-methyllignoceryl laurate, 24-methylpentacosyl laurate, 25-methylhexacosyl laurate, 26-methylheptacosyl laurate, 27-methyloctacosyl laurate, 28-methylnonacosyl laurate, 29-methyltriacontyl laurate, 30-methylhentriacontyl laurate, 31-methyldotriacontyl laurate, 32-methyltritriacontyl laurate, 33-methyltetratriacontyl laurate, 34-methylpentatriacontyl laurate, 35-methylhexatriacontyl laurate, 36-methylheptatriacontyl laurate, 37-methyloctatriacontyl laurate, 38-methylnonatriacontyl laurate and 39-methyltetracontyl laurate.

In one embodiment one or more wax esters are selected from the group comprising or consisting of anteiso-branched alkyl laurates. In one embodiment one or more wax esters are selected from the group comprising or consisting of 10-methyllauryl laurate, 11-methyltridecyl laurate, 12-methylmyristyl laurate, 13-methylpentadecyl laurate, 14-methylpalmityl laurate, 15-methylheptadecyl laurate, 16-methylstearyl laurate, 17-methylnonadecyl laurate, 18-methylarachidyl laurate, 19-methylheneicosyl laurate, 20-methylbehenyl laurate, 21-methyltricosyl laurate, 22-methyllignoceryl laurate, 23-methylpentacosyl laurate, 24-methylhexacosyl laurate, 25-methylheptacosyl laurate, 26-methyloctacosyl laurate, 27-methylnonacosyl laurate, 28-methyltriacontyl laurate, 29-methylhentriacontyl laurate, 30-methyldotriacontyl laurate, 31-methyltritriacontyl laurate, 32-methyltetratriacontyl laurate, 33-methylpentatriacontyl laurate, 34-methylhexatriacontyl laurate, 35-methylheptatriacontyl laurate, 36-methyloctatriacontyl laurate, 37-methylnonatriacontyl laurate and 38-methyltetracontyl laurate.

In one embodiment one or more wax esters are selected from the group comprising or consisting of paullinic acid-based esters. In one embodiment one or more wax esters are selected from the group comprising or consisting of lauryl eicos-13-enoate, tridecyl eicos-13-enoate, myristyl eicos-13-enoate, pentadecyl eicos-13-enoate, palmityl eicos-13-enoate, heptadecyl eicos-13-enoate, stearyl eicos-13-enoate, nonadecyl eicos-13-enoate, arachidyl eicos-13-enoate, heneicosyl eicos-13-enoate, behenyl eicos-13-enoate, tricosyl eicos-13-enoate, lignoceryl eicos-13-enoate, pentacosyl eicos-13-enoate, hexacosyl eicos-13-enoate, heptacosyl eicos-13-enoate, octacosyl eicos-13-enoate, nonacosyl eicos-13-enoate, triacontyl eicos-13-enoate, hentriacontyl eicos-13-enoate, dotriacontyl eicos-13-enoate, tritriacontyl eicos-13-enoate, tetratriacontyl eicos-13-enoate, pentatriacontyl eicos-13-enoate, hexatriacontyl eicos-13-enoate, heptatriacontyl eicos-13-enoate, octatriacontyl eicos-13-enoate, nonatriacontyl eicos-13-enoate and tetracontyl eicos-13-enoate.

In one embodiment one or more wax esters are selected from the group comprising or consisting of iso-branched alkyl paullinates. In one embodiment one or more wax esters are selected from the group comprising or consisting of 11-methyllauryl eicos-13-enoate, 12-methyltridecyl eicos-13-enoate, 13-methylmyristyl eicos-13-enoate, 14-methylpentadecyl eicos-13-enoate, 15-methylpalmityl eicos-13-enoate, 16-methylheptadecyl eicos-13-enoate, 17-methylstearyl eicos-13-enoate, 18-methylnonadecyl eicos-13-enoate, 19-methylarachidyl eicos-13-enoate, 20-methylheneicosyl eicos-13-enoate, 21-methylbehenyl eicos-13-enoate, 22-methyltricosyl eicos-13-enoate, 23-methyllignoceryl eicos-13-enoate, 24-methylpentacosyl eicos-13-enoate, 25-methylhexacosyl eicos-13-enoate, 26-methylheptacosyl eicos-13-enoate, 27-methyloctacosyl eicos-13-enoate, 28-methylnonacosyl eicos-13-enoate, 29-methyltriacontyl eicos-13-enoate, 30-methylhentriacontyl eicos-13-enoate, 31-methyldotriacontyl eicos-13-enoate, 32-methyltritriacontyl eicos-13-enoate, 33-methyltetratriacontyl eicos-13-enoate, 34-methylpentatriacontyl eicos-13-enoate, 35-methylhexatriacontyl eicos-13-enoate, 36-methylheptatriacontyl eicos-13-enoate, 37-methyloctatriacontyl eicos-13-enoate, 38-methylnonatriacontyl eicos-13-enoate and 39-methyltetracontyl eicos-13-enoate.

In one embodiment, one or more wax esters are selected from the group comprising or consisting of anteiso-branched alkyl paullinates. In one embodiment one or more wax esters are selected from the group comprising or consisting of 10-methyllauryl eicos-13-enoate, 11-methyltridecyl eicos-13-enoate, 12-methylmyristyl eicos-13-enoate, 13-methylpentadecyl eicos-13-enoate, 14-methylpalmityl eicos-13-enoate, 15-methylheptadecyl eicos-13-enoate, 16-methylstearyl eicos-13-enoate, 17-methylnonadecyl eicos-13-enoate, 18-methylarachidyl eicos-13-enoate, 19-methylheneicosyl eicos-13-enoate, 20-methylbehenyl eicos-13-enoate, 21-methyltricosyl eicos-13-enoate, 22-methyllignoceryl eicos-13-enoate, 23-methylpentacosyl eicos-13-enoate, 24-methylhexacosyl eicos-13-enoate, 25-methylheptacosyl eicos-13-enoate, 26-methyloctacosyl eicos-13-enoate, 27-methylnonacosyl eicos-13-enoate, 28-methyltriacontyl eicos-13-enoate, 29-methylhentriacontyl eicos-13-enoate, 30-methyldotriacontyl eicos-13-enoate, 31-methyltritriacontyl eicos-13-enoate, 32-methyltetratriacontyl eicos-13-enoate, 33-methylpentatriacontyl eicos-13-enoate, 34-methylhexatriacontyl eicos-13-enoate, 35-methylheptatriacontyl eicos-13-enoate, 36-methyloctatriacontyl eicos-13-enoate, 37-methylnonatriacontyl eicos-13-enoate and 38-methyltetracontyl eicos-13-enoate.

In one embodiment one or more wax esters are selected from the group comprising or consisting of gondoic acid based esters. In one embodiment one or more wax esters are selected from the group comprising or consisting of lauryl eicos-11-enoate, tridecyl eicos-11-enoate, myristyl eicos-11-enoate, pentadecyl eicos-11-enoate, palmityl eicos-11-enoate, heptadecyl eicos-11-enoate, stearyl eicos-11-enoate, nonadecyl eicos-11-enoate, arachidyl eicos-11-enoate, heneicosyl eicos-11-enoate, behenyl eicos-11-enoate, tricosyl eicos-11-enoate, lignoceryl eicos-11-enoate, pentacosyl eicos-11-enoate, hexacosyl eicos-11-enoate, heptacosyl eicos-11-enoate, octacosyl eicos-11-enoate, nonacosyl eicos-11-enoate, triacontyl eicos-11-enoate, hentriacontyl eicos-11-enoate, dotriacontyl eicos-11-enoate, tritriacontyl eicos-11-enoate, tetratriacontyl eicos-11-enoate, pentatriacontyl eicos-11-enoate, hexatriacontyl eicos-11-enoate, heptatriacontyl eicos-11-enoate, octatriacontyl eicos-11-enoate, nonatriacontyl eicos-11-enoate and tetracontyl eicos-11-enoate.

In one embodiment one or more wax esters are selected from the group comprising or consisting of iso-branched alkyl gondoates. In one embodiment one or more wax esters are selected from the group comprising or consisting of 11-methyllauryl eicos-11-enoate, 12-methyltridecyl eicos-11-enoate, 13-methylmyristyl eicos-11-enoate, 14-methylpentadecyl eicos-11-enoate, 15-methylpalmityl eicos-11-enoate, 16-methylheptadecyl eicos-11-enoate, 17-methylstearyl eicos-11-enoate, 18-methylnonadecyl eicos-11-enoate, 19-methylarachidyl eicos-11-enoate, 20-methylheneicosyl eicos-11-enoate, 21-methylbehenyl eicos-11-enoate, 22-methyltricosyl eicos-11-enoate, 23-methyllignoceryl eicos-11-enoate, 24-methylpentacosyl eicos-11-enoate, 25-methylhexacosyl eicos-11-enoate, 26-methylheptacosyl eicos-11-enoate, 27-methyloctacosyl eicos-11-enoate, 28-methylnonacosyl eicos-11-enoate, 29-methyltriacontyl eicos-11-enoate, 30-methylhentriacontyl eicos-11-enoate, 31-methyldotriacontyl eicos-11-enoate, 32-methyltritriacontyl eicos-11-enoate, 33-methyltetratriacontyl eicos-11-enoate, 34-methylpentatriacontyl eicos-11-enoate, 35-methylhexatriacontyl eicos-11-enoate, 36-methylheptatriacontyl eicos-11-enoate, 37-methyloctatriacontyl eicos-11-enoate, 38-methylnonatriacontyl eicos-11-enoate and 39-methyltetracontyl eicos-11-enoate.

In one embodiment one or more wax esters are selected from the group comprising or consisting of anteiso-branched alkyl gondoates. In one embodiment one or more wax esters are selected from the group comprising or consisting of 10-methyllauryl eicos-11-enoate, 11-methyltridecyl eicos-11-enoate, 12-methylmyristyl eicos-11-enoate, 13-methylpentadecyl eicos-11-enoate, 14-methylpalmityl eicos-11-enoate, 15-methylheptadecyl eicos-11-enoate, 16-methylstearyl eicos-11-enoate, 17-methylnonadecyl eicos-11-enoate, 18-methylarachidyl eicos-11-enoate, 19-methylheneicosyl eicos-11-enoate, 20-methylbehenyl eicos-11-enoate, 21-methyltricosyl eicos-11-enoate, 22-methyllignoceryl eicos-11-enoate, 23-methylpentacosyl eicos-11-enoate, 24-methylhexacosyl eicos-11-enoate, 25-methylheptacosyl eicos-11-enoate, 26-methyloctacosyl eicos-11-enoate, 27-methylnonacosyl eicos-11-enoate, 28-methyltriacontyl eicos-11-enoate, 29-methylhentriacontyl eicos-11-enoate, 30-methyldotriacontyl eicos-11-enoate, 31-methyltritriacontyl eicos-11-enoate, 32-methyltetratriacontyl eicos-11-enoate, 33-methylpentatriacontyl eicos-11-enoate, 34-methylhexatriacontyl eicos-11-enoate, 35-methylheptatriacontyl eicos-11-enoate, 36-methyloctatriacontyl eicos-11-enoate, 37-methylnonatriacontyl eicos-11-enoate and 38-methyltetracontyl eicos-11-enoate.

In one embodiment one or more wax esters are selected from the group comprising or consisting of erucic acid based esters. In one embodiment one or more wax esters are selected from the group comprising or consisting of lauryl docos-13-enoate, tridecyl docos-13-enoate, myristyl docos-13-enoate, pentadecyl docos-13-enoate, palmityl docos-13-enoate, heptadecyl docos-13-enoate, stearyl docos-13-enoate, nonadecyl docos-13-enoate, arachidyl docos-13-enoate, heneicosyl docos-13-enoate, behenyl docos-13-enoate, tricosyl docos-13-enoate, lignoceryl docos-13-enoate, pentacosyl docos-13-enoate, hexacosyl docos-13-enoate, heptacosyl docos-13-enoate, octacosyl docos-13-enoate, nonacosyl docos-13-enoate, triacontyl docos-13-enoate, hentriacontyl docos-13-enoate, dotriacontyl docos-13-enoate, tritriacontyl docos-13-enoate, tetratriacontyl docos-13-enoate, pentatriacontyl docos-13-enoate, hexatriacontyl docos-13-enoate, heptatriacontyl docos-13-enoate, octatriacontyl docos-13-enoate, nonatriacontyl docos-13-enoate and tetracontyl docos-13-enoate.

In one embodiment one or more wax esters are selected from the group comprising or consisting of iso-branched alkyl eruciates. In one embodiment one or more wax esters are selected from the group comprising or consisting of 11-methyllauryl docos-13-enoate, 12-methyltridecyl docos-13-enoate, 13-methylmyristyl docos-13-enoate, 14-methylpentadecyl docos-13-enoate, 15-methylpalmityl docos-13-enoate, 16-methylheptadecyl docos-13-enoate, 17-methylstearyl docos-13-enoate, 18-methylnonadecyl docos-13-enoate, 19-methylarachidyl docos-13-enoate, 20-methylheneicosyl docos-13-enoate, 21-methylbehenyl docos-13-enoate, 22-methyltricosyl docos-13-enoate, 23-methyllignoceryl docos-13-enoate, 24-methylpentacosyl docos-13-enoate, 25-methylhexacosyl docos-13-enoate, 26-methylheptacosyl docos-13-enoate, 27-methyloctacosyl docos-13-enoate, 28-methylnonacosyl docos-13-enoate, 29-methyltriacontyl docos-13-enoate, 30-methylhentriacontyl docos-13-enoate, 31-methyldotriacontyl docos-13-enoate, 32-methyltritriacontyl docos-13-enoate, 33-methyltetratriacontyl docos-13-enoate, 34-methylpentatriacontyl docos-13-enoate, 35-methylhexatriacontyl docos-13-enoate, 36-methylheptatriacontyl docos-13-enoate, 37-methyloctatriacontyl docos-13-enoate, 38-methylnonatriacontyl docos-13-enoate and 39-methyltetracontyl docos-13-enoate.

In one embodiment one or more wax esters are selected from the group comprising or consisting of anteiso-branched alkyl eruciates. In one embodiment one or more wax esters are selected from the group comprising or consisting of 10-methyllauryl docos-13-enoate, 11-methyltridecyl docos-13-enoate, 12-methylmyristyl docos-13-enoate, 13-methylpentadecyl docos-13-enoate, 14-methylpalmityl docos-13-enoate, 15-methylheptadecyl docos-13-enoate, 16-methylstearyl docos-13-enoate, 17-methylnonadecyl docos-13-enoate, 18-methylarachidyl docos-13-enoate, 19-methylheneicosyl docos-13-enoate, 20-methylbehenyl docos-13-enoate, 21-methyltricosyl docos-13-enoate, 22-methyllignoceryl docos-13-enoate, 23-methylpentacosyl docos-13-enoate, 24-methylhexacosyl docos-13-enoate, 25-methylheptacosyl docos-13-enoate, 26-methyloctacosyl docos-13-enoate, 27-methylnonacosyl docos-13-enoate, 28-methyltriacontyl docos-13-enoate, 29-methylhentriacontyl docos-13-enoate, 30-methyldotriacontyl docos-13-enoate, 31-methyltritriacontyl docos-13-enoate, 32-methyltetratriacontyl docos-13-enoate, 33-methylpentatriacontyl docos-13-enoate, 34-methylhexatriacontyl docos-13-enoate, 35-methylheptatriacontyl docos-13-enoate, 36-methyloctatriacontyl docos-13-enoate, 37-methylnonatriacontyl docos-13-enoate and 38-methyltetracontyl docos-13-enoate.

In one embodiment one or more wax esters are selected from the group comprising or consisting of nervonic acid based esters. In one embodiment one or more wax esters are selected from the group comprising or consisting of lauryl tetracos-15-enoate, tridecyl tetracos-15-enoate, myristyl tetracos-15-enoate, pentadecyl tetracos-15-enoate, palmityl tetracos-15-enoate, heptadecyl tetracos-15-enoate, stearyl tetracos-15-enoate, nonadecyl tetracos-15-enoate, arachidyl tetracos-15-enoate, heneicosyl tetracos-15-enoate, behenyl tetracos-15-enoate, tricosyl tetracos-15-enoate, lignoceryl tetracos-15-enoate, pentacosyl tetracos-15-enoate, hexacosyl tetracos-15-enoate, heptacosyl tetracos-15-enoate, octacosyl tetracos-15-enoate, nonacosyl tetracos-15-enoate, triacontyl tetracos-15-enoate, hentriacontyl tetracos-15-enoate, dotriacontyl tetracos-15-enoate, tritriacontyl tetracos-15-enoate, tetratriacontyl tetracos-15-enoate, pentatriacontyl tetracos-15-enoate, hexatriacontyl tetracos-15-enoate, heptatriacontyl tetracos-15-enoate, octatriacontyl tetracos-15-enoate, nonatriacontyl tetracos-15-enoate and tetracontyl tetracos-15-enoate.

In one embodiment one or more wax esters are selected from the group comprising or consisting of iso-branched alkyl nervonates. In one embodiment one or more wax esters are selected from the group comprising or consisting of 11-methyllauryl tetracos-15-enoate, 12-methyltridecyl tetracos-15-enoate, 13-methylmyristyl tetracos-15-enoate, 14-methylpentadecyl tetracos-15-enoate, 15-methylpalmityl tetracos-15-enoate, 16-methylheptadecyl tetracos-15-enoate, 17-methylstearyl tetracos-15-enoate, 18-methylnonadecyl tetracos-15-enoate, 19-methylarachidyl tetracos-15-enoate, 20-methylheneicosyl tetracos-15-enoate, 21-methylbehenyl tetracos-15-enoate, 22-methyltricosyl tetracos-15-enoate, 23-methyllignoceryl tetracos-15-enoate, 24-methylpentacosyl tetracos-15-enoate, 25-methylhexacosyl tetracos-15-enoate, 26-methylheptacosyl tetracos-15-enoate, 27-methyloctacosyl tetracos-15-enoate, 28-methylnonacosyl tetracos-15-enoate, 29-methyltriacontyl tetracos-15-enoate, 30-methylhentriacontyl tetracos-15-enoate, 31-methyldotriacontyl tetracos-15-enoate, 32-methyltritriacontyl tetracos-15-enoate, 33-methyltetratriacontyl tetracos-15-enoate, 34-methylpentatriacontyl tetracos-15-enoate, 35-methylhexatriacontyl tetracos-15-enoate, 36-methylheptatriacontyl tetracos-15-enoate, 37-methyloctatriacontyl tetracos-15-enoate, 38-methylnonatriacontyl tetracos-15-enoate and 39-methyltetracontyl tetracos-15-enoate.

In one embodiment one or more wax esters are selected from the group comprising or consisting of anteiso-branched alkyl nervonates. In one embodiment one or more wax esters are selected from the group comprising or consisting of 10-methyllauryl tetracos-15-enoate, 11-methyltridecyl tetracos-15-enoate, 12-methylmyristyl tetracos-15-enoate, 13-methylpentadecyl tetracos-15-enoate, 14-methylpalmityl tetracos-15-enoate, 15-methylheptadecyl tetracos-15-enoate, 16-methylstearyl tetracos-15-enoate, 17-methylnonadecyl tetracos-15-enoate, 18-methylarachidyl tetracos-15-enoate, 19-methylheneicosyl tetracos-15-enoate, 20-methylbehenyl tetracos-15-enoate, 21-methyltricosyl tetracos-15-enoate, 22-methyllignoceryl tetracos-15-enoate, 23-methylpentacosyl tetracos-15-enoate, 24-methylhexacosyl tetracos-15-enoate, 25-methylheptacosyl tetracos-15-enoate, 26-methyloctacosyl tetracos-15-enoate, 27-methylnonacosyl tetracos-15-enoate, 28-methyltriacontyl tetracos-15-enoate, 29-methylhentriacontyl tetracos-15-enoate, 30-methyldotriacontyl tetracos-15-enoate, 31-methyltritriacontyl tetracos-15-enoate, 32-methyltetratriacontyl tetracos-15-enoate, 33-methylpentatriacontyl tetracos-15-enoate, 34-methylhexatriacontyl tetracos-15-enoate, 35-methylheptatriacontyl tetracos-15-enoate, 36-methyloctatriacontyl tetracos-15-enoate, 37-methylnonatriacontyl tetracos-15-enoate and 38-methyltetracontyl tetracos-15-enoate.

In one embodiment one or more wax esters are selected from the group comprising or consisting of linoleic acid based esters. In one embodiment one or more wax esters are selected from the group comprising or consisting of lauryl linoleate, tridecyl linoleate, myristyl linoleate, pentadecyl linoleate, palmityl linoleate, heptadecyl linoleate, stearyl linoleate, nonadecyl linoleate, arachidyl linoleate, heneicosyl linoleate, behenyl linoleate, tricosyl linoleate, lignoceryl linoleate, pentacosyl linoleate, hexacosyl linoleate, heptacosyl linoleate, octacosyl linoleate, nonacosyl linoleate, triacontyl linoleate, hentriacontyl linoleate, dotriacontyl linoleate, tritriacontyl linoleate, tetratriacontyl linoleate, pentatriacontyl linoleate, hexatriacontyl linoleate, heptatriacontyl linoleate, octatriacontyl linoleate, nonatriacontyl linoleate and tetracontyl linoleate.

In one embodiment one or more wax esters are selected from the group comprising or consisting of iso-branched alkyl linoleates. In one embodiment one or more wax esters are selected from the group comprising or consisting of 11-methyllauryl linoleate, 12-methyltridecyl linoleate, 13-methylmyristyl linoleate, 14-methylpentadecyl linoleate, 15-methylpalmityl linoleate, 16-methylheptadecyl linoleate, 17-methylstearyl linoleate, 18-methylnonadecyl linoleate, 19-methylarachidyl linoleate, 20-methylheneicosyl linoleate, 21-methylbehenyl linoleate, 22-methyltricosyl linoleate, 23-methyllignoceryl linoleate, 24-methylpentacosyl linoleate, 25-methylhexacosyl linoleate, 26-methylheptacosyl linoleate, 27-methyloctacosyl linoleate, 28-methylnonacosyl linoleate, 29-methyltriacontyl linoleate, 30-methylhentriacontyl linoleate, 31-methyldotriacontyl linoleate, 32-methyltritriacontyl linoleate, 33-methyltetratriacontyl linoleate, 34-methylpentatriacontyl linoleate, 35-methylhexatriacontyl linoleate, 36-methylheptatriacontyl linoleate, 37-methyloctatriacontyl linoleate, 38-methylnonatriacontyl linoleate and 39-methyltetracontyl linoleate.

In one embodiment one or more wax esters are selected from the group comprising or consisting of anteiso-branched alkyl linoleates. In one embodiment one or more wax esters are selected from the group comprising or consisting of 10-methyllauryl linoleate, 11-methyltridecyl linoleate, 12-methylmyristyl linoleate, 13-methylpentadecyl linoleate, 14-methylpalmityl linoleate, 15-methylheptadecyl linoleate, 16-methylstearyl linoleate, 17-methylnonadecyl linoleate, 18-methylarachidyl linoleate, 19-methylheneicosyl linoleate, 20-methylbehenyl linoleate, 21-methyltricosyl linoleate, 22-methyllignoceryl linoleate, 23-methylpentacosyl linoleate, 24-methylhexacosyl linoleate, 25-methylheptacosyl linoleate, 26-methyloctacosyl linoleate, 27-methylnonacosyl linoleate, 28-methyltriacontyl linoleate, 29-methylhentriacontyl linoleate, 30-methyldotriacontyl linoleate, 31-methyltritriacontyl linoleate, 32-methyltetratriacontyl linoleate, 33-methylpentatriacontyl linoleate, 34-methylhexatriacontyl linoleate, 35-methylheptatriacontyl linoleate, 36-methyloctatriacontyl linoleate, 37-methylnonatriacontyl linoleate, and 38-methyltetracontyl linoleate.

In one embodiment one or more wax esters are selected from the group comprising or consisting of linolenic acid based esters. In one embodiment one or more wax esters are selected from the group comprising or consisting of lauryl linolenate, tridecyl linolenate, myristyl linolenate, pentadecyl linolenate, palmityl linolenate, heptadecyl linolenate, stearyl linolenate, nonadecyl linolenate, arachidyl linolenate, heneicosyl linolenate, behenyl linolenate, tricosyl linolenate, lignoceryl linolenate, pentacosyl linolenate, hexacosyl linolenate, heptacosyl linolenate, octacosyl linolenate, nonacosyl linolenate, triacontyl linolenate, hentriacontyl linolenate, dotriacontyl linolenate, tritriacontyl linolenate, tetratriacontyl linolenate, pentatriacontyl linolenate, hexatriacontyl linolenate, heptatriacontyl linolenate, octatriacontyl linolenate, nonatriacontyl linolenate and tetracontyl linolenate.

In one embodiment one or more wax esters are selected from the group comprising or consisting of iso-branched alkyl linolenates. In one embodiment one or more wax esters are selected from the group comprising or consisting of 11-methyllauryl linolenate, 12-methyltridecyl linolenate, 13-methylmyristyl linolenate, 14-methylpentadecyl linolenate, 15-methylpalmityl linolenate, 16-methylheptadecyl linolenate, 17-methylstearyl linolenate, 18-methylnonadecyl linolenate, 19-methylarachidyl linolenate, 20-methylheneicosyl linolenate, 21-methylbehenyl linolenate, 22-methyltricosyl linolenate, 23-methyllignoceryl linolenate, 24-methylpentacosyl linolenate, 25-methylhexacosyl linolenate, 26-methylheptacosyl linolenate, 27-methyloctacosyl linolenate, 28-methylnonacosyl linolenate, 29-methyltriacontyl linolenate, 30-methylhentriacontyl linolenate, 31-methyldotriacontyl linolenate, 32-methyltritriacontyl linolenate, 33-methyltetratriacontyl linolenate, 34-methylpentatriacontyl linolenate, 35-methylhexatriacontyl linolenate, 36-methylheptatriacontyl linolenate, 37-methyloctatriacontyl linolenate, 38-methylnonatriacontyl linolenate and 39-methyltetracontyl linolenate.

In one embodiment one or more wax esters are selected from the group comprising or consisting of anteiso-branched alkyl linolenates. In one embodiment one or more wax esters are selected from the group comprising or consisting of 10-methyllauryl linolenate, 11-methyltridecyl linolenate, 12-methylmyristyl linolenate, 13-methylpentadecyl linolenate, 14-methylpalmityl linolenate, 15-methylheptadecyl linolenate, 16-methylstearyl linolenate, 17-methylnonadecyl linolenate, 18-methylarachidyl linolenate, 19-methylheneicosyl linolenate, 20-methylbehenyl linolenate, 21-methyltricosyl linolenate, 22-methyllignoceryl linolenate, 23-methylpentacosyl linolenate, 24-methylhexacosyl linolenate, 25-methylheptacosyl linolenate, 26-methyloctacosyl linolenate, 27-methylnonacosyl linolenate, 28-methyltriacontyl linolenate, 29-methylhentriacontyl linolenate, 30-methyldotriacontyl linolenate, 31-methyltritriacontyl linolenate, 32-methyltetratriacontyl linolenate, 33-methylpentatriacontyl linolenate, 34-methylhexatriacontyl linolenate, 35-methylheptatriacontyl linolenate, 36-methyloctatriacontyl linolenate, 37-methylnonatriacontyl linolenate, and 38-methyltetracontyl linolenate.

In one embodiment, the one or more wax esters are selected from palmityl oleate, stearyl oleate, arachidyl oleate (AO), behenyl oleate (BO), lignoceryl oleate, hexocosanyl oleate, 24-methylpentacosanyl oleate, palmityl palmitoleate, stearyl palmitoleate, arachidyl palmitoleate, behenyl palmitolate, lignoceryl palmitoleate, 24-methylpentacosanyl palmitoleate, palmityl linoleate, stearyl linoleate, arachidyl linoleate, behenyl palmitolate, lignoceryl linoleate, 24-methylpentacosanyl linoleate, palmityl linolenate, stearyl linolenate, arachidyl linolenate, behenyl palmitolate, lignoceryl linolenate, and 24-methylpentacosanyl linolenate.

In particular embodiments, the wax ester is selected from the group consisting of behenyl oleate and arachidyl laurate.

More particularly, the wax ester is behenyl oleate.

In alternative embodiments, the wax ester is arachidyl laurate

As used herein, “alkyl” as a group or part of a group refers to a straight or branched aliphatic hydrocarbon group. Exemplary alkyl groups include, but are not limited to, methyl, ethyl, propyl, butyl, pentyl, and hexyl.

As used herein, “alkenyl” as a group or part of a group refers to an aliphatic hydrocarbon group which may be straight or branched and which contains at least one carbon-carbon double bond. Exemplary alkenyl groups include, but are not limited to, ethenyl, propenyl, butenyl, pentenyl, hexenyl, heptenyl, octenyl and nonenyl.

As used herein, “alkynyl” as a group or part of a group refers to an aliphatic hydrocarbon group which may be straight or branched and which contains a carbon-carbon triple bond. Exemplary alkynyl groups include, but are not limited to, ethynyl and propynyl.

Isomeric forms including diastereoisomers, enantiomers, tautomers, and geometrical isomers are included within the scope of the compounds presented in the present disclosure. Also solvated, nonsolvated, hydrated, non-hydrated, charged and neutral forms are included within the scope of said compounds.

As used herein “a structural analogue” refers to a compound having a structure similar to a compound described in the present disclosure but differing from it in respect to a certain component such as one or more atoms, functional groups, or substructures, which are replaced with other atoms, groups, or substructures. In one embodiment structural analogs are isoelectronic analogues and/or functional analogues.

The biophysical properties of the specific FAHFAs (or structural analogues thereof) or wax esters (or structural analogues thereof) used in the examples of the present disclosure are representative of the lipids class FAHFAs (including OAHFAs) or wax esters, respectively, on the whole.

In one embodiment of the invention, the one or more wax esters (such as arachidyl oleate, AO) of the composition are in the liquid state at the physiological conditions, and/or one or more wax esters (such as behenyl oleate, BO) of the composition are in the solid state at the physiological conditions. In a very specific embodiment one or more of the wax esters, or all wax esters of the composition are in the solid state at the physiological conditions. In one embodiment, the melting point(s) of one or more or all wax ester(s) of the composition is (are) below, equal or above the temperature of the ocular surface. The composition of the present invention can comprise any amounts of FAHFAs (or structural analogues thereof) and wax esters (or structural analogues thereof).

In one embodiment in the composition the molar ratio of one or more FAHFAs and/or structural analogues thereof to one or more wax esters and/or structural analogues thereof is 1:1 or less, about 1:1-1:100, or 1:2, 1:3, 1:4, 1:5, 1:6, 1:7, 1:8, 1:9 or 1:10, 1:20, 1:30, 1:40, 1:50, 1:60, 1:70, 1:80, 1:90. In one embodiment the molar ratio of one or more FAHFAs and/or structural analogues thereof to one or more wax esters and/or structural analogues thereof is more than 1:1, about 100:1-1:1, about 3:2-5:1 or 90:1, 80:1, 70:1, 60:1, 50:1, 40:1, 30:1, 20:1, 10:1, 9:1, 8:1, 7:1, 6:1, 5:1, 4:1, 3:1, or 2:1.

As described herein, certain combinations of FAHFA and wax ester have highly advantageous properties in terms of their evaporation resistance at aqueous surfaces (including, in particular, at the ocular surface). In particular, mixtures of FAHFAs (including 20-OAHFA and 18:1/18:0-OAHFA) and wax esters (including BO and AL) wherein the amount of the wax ester is equal to or higher than the amount of the FAHFA have been shown to achieve evaporation resistance values that are considerably higher than the individual components alone, and the tear forming lipid layer in healthy subjects ((9-13 s/cm) (Iwata, S., et al. 1969, Invest Ophthalmol Vis Sci, 8, 613-619; Peng, C., et al. 2014, Ind Eng Cham Res, 53, 18130-18139)).

Thus, in a particular embodiment, the amount of the wax ester (or structural analogue thereof) is equal to or higher than the amount of the FAHFA (or structural analogue thereof) i.e the ratio of the FAHFA or structural analogue thereof (e.g. FAHFA) to wax ester or structural analogue thereof (e.g. wax ester) in the composition is 1:1 or less (FAHFA:wax ester) (for example from 1:1 to 1:9, such as from 1:1 to 1:3 (e.g. about 1:1)).

In a further particular embodiment, the ratio of the FAHFA or structural analogue thereof (e.g. FAHFA) to wax ester or structural analogue thereof (e.g. wax ester) is about 1:1.

In one embodiment the composition comprises at least 0.001, 0.005, 0.01, 0.05, 0.1, 0.5, 1, 2, 3, 4, 5, 6, 7, 8, 9, 10, 15, 20, 25, 30, 35, 40, 45, or 50% w/v or wt % FAHFAs and/or structural analogues thereof, and/or wax esters and/or structural analogues thereof. In one embodiment the composition comprises at least 0.001, 0.005, 0.01, 0.05, 0.1, 0.5, 1, 2, 3, 4, 5, 6, 7, 8, 9, 10, 15, 20, 25, 30, 35, 40, 45, or 50% w/v or wt % lipids. As used herein % w/v (i.e. % weight per volume) refers to mass concentration of a solution. As used herein, wt % (i.e. % mass of solute per mass of the solution) refers to weight percent of a solution.

In one embodiment, the composition comprises or consists of one or more FAHFAs selected from 12-OAHFA (12-(oleoyloxy)dodecanoic acid), 15-OAHFA (15-(oleoyxy)pentadecanoic acid), 20-OAHFA (20-(oleoyloxy)eicosanoic acid), 22-OAHFA (22-(oleoyloxy)docosanoic acid), 20:1-OAHFA ((12Z)-20-(oleoyloxy)eicos-12-enoic acid) and/or 29:1-OAHFA ((21Z)-29-(oleoyloxy)nonacos-21-enoic acid) and one or more wax esters selected from palmityl oleate, stearyl oleate, arachidyl oleate (AO), behenyl oleate (BO), lignoceryl oleate, hexocosanyl oleate, 24-methylpentacosanyl oleate, palmityl palmitoleate, stearyl palmitoleate, arachidyl palmitoleate, behenyl palmitolate, lignoceryl palmitoleate, 24-methylpentacosanyl palmitoleate, palmityl linoleate, stearyl linoleate, arachidyl linoleate, behenyl palmitolate, lignoceryl linoleate, 24-methylpentacosanyl linoleate, palmityl linolenate, stearyl linolenate, arachidyl linolenate, behenyl palmitolate, lignoceryl linolenate, and 24-methylpentacosanyl linolenate. In one embodiment, the composition comprises or consists of one or more FAHFAs selected from selected from 20-OAHFA (20-(oleoyloxy)eicosanoic acid), 20:1-OAHFA ((12Z)-20-(oleoyloxy)eicos-12-enoic acid) and 29:1-OAHFA ((21Z)-29-(oleoyloxy)nonacos-21-enoic acid) and one or more wax esters selected from arachidyl oleate, behenyl oleate, hexocosanyl oleate and 24-methylpentacosanyl oleate.

In a particular embodiment, the FAHFA in the composition is selected from the group consisting of 20-(oleoyloxy)eicosanoic acid, 18-(oleoyloxy)stearic acid and 20-(palmitoleoyloxy)eicosanoic acid and the wax ester is selected from the group consisting of behenyl oleate and arachidyl laurate. In such compositions, the ratio of FAHFA to wax ester is 1:1 or less, preferably from 1:1 to 1:9 (FAHFA:wax ester) (for example from 1:1 to 1:3, e.g. about 1:1). In particular embodiments, such compositions do not comprise any other FAHFA or wax ester components.

In further particular embodiments, the FAHFA is selected from the group consisting of 20-(oleoyloxy)eicosanoic acid and 18-(oleoyloxy)stearic acid and the wax ester is selected from the group consisting of behenyl oleate and arachidyl laurate. In such compositions, the ratio of FAHFA to wax ester is 1:1 or less, preferably from 1:1 to 1:9 (FAHFA:wax ester) (for example from 1:1 to 1:3, e.g. about 1:1). In particular embodiments, such compositions do not comprise any other FAHFA or wax ester components.

In a more particular embodiment, the FAHFA is 20-(oleoyloxy)eicosanoic acid and the wax ester is selected from the group consisting of behenyl oleate and arachidyl laurate. In a yet more particular embodiment, the FAHFA is 20-(oleoyloxy)eicosanoic acid and the wax ester is behenyl oleate. In such compositions, the ratio of 20-(oleoyloxy)eicosanoic acid to wax ester is 1:1 or less, preferably from 1:1 to 1:9 (FAHFA:wax ester) (for example, 1:1 to 1:3 (e.g. 1:1)). In particular embodiments, such compositions also do not comprise any other FAHFA or wax ester components.

In a further particular embodiment, the FAHFA is 18-(oleoyloxy)stearic acid and the wax ester is selected from the group consisting of behenyl oleate and arachidyl laurate. In such compositions, the ratio of 18-(oleoyloxy)stearic acid to wax ester is 1:1 or less, preferably from 1:1 to 1:9 (FAHFA:wax ester) (for example, 1:1 to 1:3 (preferably about 1:1). In particular embodiments, such compositions also do not comprise any other FAHFA or wax ester components.

In a further particular embodiment, the FAHFA is 20-(palmitoleoyloxy)eicosanoic acid and the wax ester is selected from the group consisting of behenyl oleate and arachidyl laurate. In such compositions, the ratio of 20-(palmitoleoyloxy)eicosanoic acid to wax ester is 1:1 (FAHFA:wax ester) or less, preferably about 1:1. In particular embodiments, such compositions also do not comprise any other FAHFA or wax ester components.

In one embodiment of the invention the FAHFA (or a structural analogue thereof) and the wax ester (or a structural analogue thereof) can be in separate pre-compositions to be applied or administered at the same time as a combination to a target or on a target of interest, or the FAHFA (or a structural analogue thereof) and the wax ester (or a structural analogue thereof) can be comprised in a single composition which can be e.g. a ready to use composition or a composition to be complemented before use. For example, a diluent or any other additive can be added to the composition before its use.

The composition of the present invention may also comprise any other lipids or biologically or therapeutically effective agents than FAHFAs (or a structural analogue thereof) and wax esters (or a structural analogue thereof). However, in one embodiment the composition does not comprise other lipids or biologically or therapeutically effective agents than FAHFAs (or a structural analogue thereof) and wax esters (or a structural analogue thereof). As used herein “biologically or therapeutically effective agents” refer to any agents causing a biological or therapeutic effect in the target of interest. Increase of evaporation resistance, decrease of micro-organisms and alleviation of symptoms are just some examples of said effects.

The composition of the present invention optionally comprises one or more additives and/or any components normally found in corresponding products. Said one or more additives can be selected e.g. from the group comprising or consisting of solvents, diluents, carriers, buffers, excipients, adjuvants, carrier media, antiseptics, fillers, stabilizers, thickening agents, emulsifiers, disintegrants, lubricants, and binders, and any combination thereof; and/or one or more additives can be selected e.g. from the group comprising or consisting of pharmaceutically acceptable solvents, diluents, carriers, buffers, excipients, adjuvants, carrier media, antiseptics, fillers, stabilizers, thickening agents, emulsifiers, disintegrants, lubricants, and binders, and any combination thereof. In one embodiment one or more pharmaceutically acceptable excipients are ophthalmologically acceptable excipients selected from the group consisting of polyethylene glycol, propylene glycol, glycerin, polyvinyl alcohol, povidone, polysorbate 80, hydroxypropyl methylcellulose, carmellose, carbomer 980, sodium hyaluronate and dextran.

In one embodiment the composition comprises one or more of the following: a pH adjusting agent (e.g. NaOH and/or HCl), buffering agent (e.g. phosphate and/or borate), isotonicity adjusting agent (e.g. NaCl, trehalose), viscosity increasing excipient (e.g. hydroxypropyl methylcellulose, carmellose, carbomer and/or sodium hyaluronate), preservative (e.g. benzalkoniumchloride), stabilizer (e.g. polysorbate and/or glycerin), oil phase (e.g. vaselin, paraffin, castor oil and/or mineral oil), water.

In one embodiment the composition comprises of 0.1-5% w/v one or more FAHFAs and/or structural analogues thereof, 0.1-10% w/v one or more wax esters and/or structural analogues thereof, 0-2% w/v Miglyol 812, 1-8% w/v Tween 20, 0.25-4% w/v Kolliphor® EL, 0.25-5% w/v Span 80, 1-3% w/v glycerin, and 97.3-76% w/v ultrapure water.

In a particular aspect of the invention, there is provided a composition comprising, (or consisting essentially of or consisting of) a combination of:
i. an O-Acyl-w-hydroxy fatty acid, selected from the group consisting of 20-(oleoyloxy)eicosanoic acid, 18-(oleoyloxy)stearic acid and 20-(palmitoleoyloxy)eicosanoic acid; and
ii. a wax ester, selected from the group consisting of behenyl oleate and arachidyl laurate;
   optionally, wherein the ratio of the O-Acyl-w-hydroxy fatty acid to wax ester is at least 1:1, such as from 1:1 to 1:9 (0-Acyl-w-hydroxy fatty acid to wax ester) (for example 1:1 to 1:3 (e.g. 1:1)).

In a particular embodiment of this aspect of the invention, the O-Acyl-w-hydroxy fatty acid is 20-(oleoyloxy)eicosanoic acid and the wax ester is selected from the group consisting of behenyl oleate and arachidyl laurate. More particularly, the wax ester is behenyl oleate.

In a further embodiment of this aspect of the invention, the O-Acyl-w-hydroxy fatty acid is 18-(oleoyloxy)stearic acid and the wax ester is selected from the group consisting of behenyl oleate and arachidyl laurate. In such compositions, the ratio of the O-Acyl-w-hydroxy fatty acid:wax ester is preferably about 1:1.

In a further embodiment of this aspect of the invention, the O-Acyl-w-hydroxy fatty acid is 20-(palmitoleoyloxy)eicosanoic acid and the wax ester is selected from the group consisting of behenyl oleate and arachidyl laurate. In such compositions, the ratio of the O-Acyl-w-hydroxy fatty acid:wax ester is preferably about 1:1.

In particular embodiments of this aspect of the invention, the composition does not comprise any further O-Acyl-w-hydroxy fatty acid or wax ester components.

The composition of the present invention may be in any form, such as in a liquid, semisolid or solid form, optionally suitable for administration. A formulation can be selected from a group comprising or consisting of solutions, emulsions, suspensions, spray, powder, tablets, pellets and capsules. In one embodiment the composition is an oil-in-water emulsion. If the FAHFAs (or a structural analogue thereof) and wax esters (or a structural analogue thereof) are in separate pre-compositions, a formulation for each composition can be selected independently from the above lists of formulations.

The composition of the present invention can be any kind of composition such as a pharmaceutical composition. In one embodiment the composition of the present invention is an eye drop; eye lotion; liquid, semi-solid or solid eye preparation; or powder (e.g. for eye drops or lotions, e.g. lyophilized to powder). Eyedrops can be e.g. sterile aqueous or oily solutions, emulsions or suspensions. Sterile eye preparations can be intended for administration upon the eyeball and/or to the conjunctiva, or for the insertion in the conjunctival sac. Semi-solid eye preparations can be e.g. sterile ointments, creams or gels.

In one embodiment the composition of the present invention is a homogeneous or heterogenous amphiphilic composition.

The present invention also concerns a method of preparing the composition of the present invention, wherein said method comprises combining or mixing a FAHFA or a structural analogue thereof and a wax ester or a structural analogue thereof, and optionally one or more additives.

Combining can be carried out by any method known to a person skilled in the art, for example by combining dried form of a FAHFA and a wax ester and optionally thereafter diluting the combination e.g. with one or more additives. Mixing can be carried out e.g. with any method or tools including but not limited to a stirrer, mixer, vortex, and agitation. For example, the composition of the present invention can have been combined or mixed from the point of manufacture and optionally does not require any dilution or further processing before use. In one embodiment the diluent or additive; the FAHFA or structural analogue thereof; and/or the wax ester or a structural analogue thereof are separated from the point of manufacture and in storage. For example, in one embodiment the lipids are not in fluid contact until just before use of the composition.

The compositions of the present invention may be produced by any conventional processes known in the art.

In one embodiment the method of preparing the composition of the present invention further comprises preparing or synthesizing the FAHFA or a structural analogue thereof e.g. before mixing it with the wax ester or a structural analogue thereof; and/or preparing or synthesizing the wax ester or a structural analogue thereof e.g. before mixing it with the FAHFA or a structural analogue thereof.

The FAHFA or a structural analogue thereof and/or the wax ester or a structural analogue thereof may be produced by any conventional process known in the art. In one embodiment the FAHFA or a structural analogue thereof is synthesized or has been synthesized by the methods described in the examples below or by the methods previously reported e.g. as in Bland, H., C., et al. (2019, Langmuir, Vol. 35, 3545-3552), Viitaja, T., et. al. (2021, J. Org. Chem., 86, 4965-4976) or Hancock, S., E., (2018, J. Lipid Res., 59, 1510-1518). In one embodiment the wax ester or a structural analogue thereof is synthesized or has been synthesized by the methods described in the examples below or by typical esterification protocols.

### Therapeutic and Non-Therapeutic Uses of the Compositions

The present invention concerns a non-therapeutic method of preventing evaporation of water. According to the present invention the composition can be applied on a surface to be protected from evaporation or to a material to be protected from evaporation. Suitable surfaces to be protected from evaporation of water include but are not limited to water reservoirs, artificial lakes, water storages, aqueducts, canals, and watering ponds. Suitable materials to be protected from evaporation include but are not limited to membranes and filters. Amounts for applying the composition on the surface or to the material can be determined readily by those skilled in the art.

Also, the present invention concerns the composition of the present invention for use as a medicament or for use in the treatment of dry eye disease and/or Meibomian gland dysfunction, or for use in alleviation of eye discomfort. The present invention further concerns a therapeutic method of preventing or retarding evaporation of water as well as a method of treating dry eye disease and/or Meibomian gland dysfunction, or alleviating eye discomfort.

As used herein “dry eye disease” refers to the condition of having dry eyes. Said condition may occur when the tears are not able to provide adequate lubrication for the eyes. For example, dry eyes may occur if sufficient amounts of tears are not produced or if the produced tears are of poor quality. In one embodiment the dry eye disease may be selected from allergic conjunctivitis, infective keratoconjunctivitis, or allergic conjunctivitis after infective keratoconjunctivitis. As used herein “Meibomian gland dysfunction” refers to the condition where the Meibomian glands do not secrete enough oil into the tears or when the oil they secrete is of poor quality. As used herein “eye discomfort” refers to the condition where there is lack of ease in the eyes, e.g. there is one or more of the following in one or both eyes: stinging, irritation, soreness, dryness, itching, scratchiness, aching, pain, heaviness, tenderness, tiredness, photosensitivity, sensitivity to wind, secretion, tearing, watering, discharge, mucus, crusting, heat, warmth, coldness, redness, tingling, blinking.

According to the present invention the composition can be applied on a surface to be protected from evaporation or administered to a subject in need thereof, such as to the surface of an eye of a subject.

Amounts and regimens for therapeutic administration of the composition can be determined readily by those skilled in the clinical art of treating eye diseases or disorders. Generally, the dosage of the composition of the present invention varies depending on multiple factors such as age, gender, other possible treatments, a disorder in question and severity of the symptoms. Therapeutically effective amounts of the composition can be empirically determined using art-recognized dose-escalation and dose-response assays. The composition of the present invention can be administered e.g. at doses of 0.001-0.5 ml. For example, use of eye drops are well known to a person skilled in the art and various dosage instructions are available. Monitoring the progression of the therapy or patient side effects can provide additional guidance for an optimal dosing regimen.

In one embodiment of the invention a subject is a human, a child, an adolescent or an adult. Also, any animal or mammal, such as a pet, domestic animal or production animal, may be a subject of the present invention. A subject is in a need of a treatment or prevention of dry eye disease and/or Meibomian gland dysfunction, or alleviation of eye discomfort.

As used herein, the term “treatment” or “treating” refers to administration of the composition to a subject for purposes which include not only complete cure but also amelioration, delay or alleviation of disorders or symptoms related to a disorder in question. Therapeutically effective amount of the composition refers to an amount with which the harmful effects of a disorder such as dry eye disease, Meibomian gland dysfunction, or eye discomfort, are, at a minimum, ameliorated. The harmful effects can include but are not limited to dry eyes, irritation of eyes, redness of eyes, discharge of eyes, easily fatigued eyes, and impaired vision such as blurred vision. Therapeutically effective amount can comprise an amount effective to reduce, delay or stop at least some of the harmful effects. The effects of the composition of the present invention may be either short term or long-term effects.

Before classifying a subject as suitable for the therapy of the present invention, the clinician may for example study any symptoms or assay any disease markers of the subject. Based on the results deviating from the normal, the clinician may suggest the treatment of the present invention for the subject. In one embodiment the subject to be administered with the composition of the present invention has been diagnosed with dry eye disease and/or Meibomian gland dysfunction.

Any conventional method may be used for administration of the composition to a subject. The route of administration depends on the formulation or form of the composition, the disorder, the patient, and other factors. In one embodiment of the invention, the composition is administered to the surface of the eye (on the eyeball), to the conjunctiva, to the conjunctival sac, or through an eyelid.

A desired dosage can be administered in one or more doses at suitable intervals to obtain the desired results. Only one administration of the composition of the present invention may have therapeutic effects, but specific embodiments of the invention require several administrations during the treatment period. In one embodiment the composition is (to be) administered one or several times during the treatment period. For example, administration may take place from 1 to 10 times or 1 to 5 times (such as 1-2 times) in one day e.g. during the treatment period. The length of the treatment period may vary, and may last e.g. 1 week-12 months, 1-10 years or even more.

The composition of the present invention may be used alone or together with one or more other compositions or agents, such as therapeutic agents, for treating dry eye disease and/or Meibomian gland dysfunction, or alleviating eye discomfort. Administration of the composition of the present invention and said other composition(s) or agent(s) can be simultaneous, separate or sequential. The administration of the composition of the present invention can also be combined to other forms of therapy, such as surgery, and may be more effective than either one alone. In one embodiment the composition of the present invention is utilized as the only therapeutically active agent.

The efficacy of the present invention for treating dry eye disease and/or Meibomian gland dysfunction, or alleviating eye discomfort can be studied in an in vivo dry eye model using rats or rabbits. The dry eye disease condition can first be induced, for example by treatment with benzalkonium chloride, and a formulation described in the present invention can then be administered in a single or in multiple doses, and the progression of DED monitored by analyzing the tear breakup time as well as conducting a thorough biomicroscopic evaluation of the effects followed by vital staining. At the end of the experiment, the eyes can be collected, and the inflammation markers determined by immunohistochemistry.

The efficacy of the present invention for retarding the evaporation of water from water reservoirs and artificial lakes can be studied using either the techniques described in section 1.2 below or by conducting field experiments with customized water containers equipped with heat, rain and wind sensors.

Any method or use of the invention may be executed either in vivo, ex vivo or in vitro.

It will be obvious to a person skilled in the art that, as the technology advances, the inventive concept can be implemented in various ways. The invention and its embodiments are not limited to the examples described below but may vary within the scope of the claims.

Further particular embodiments of the invention are defined in the following numbered Paragraphs.

Paragraph 1. A composition comprising a combination of a fatty acid ester of a hydroxy fatty acid (FAHFA) or a structural analogue thereof and a wax ester or a structural analogue thereof, and optionally one or more additives.

Paragraph 2. The composition of paragraph 1 consisting of a combination of a FAHFA or a structural analogue thereof and a wax ester or a structural analogue thereof, and optionally one or more additives.

Paragraph 3. The composition of any one of Paragraph 1 or Paragraph 2, wherein the composition is a pharmaceutical composition.

Paragraph 4. The composition of any of one of Paragraphs 1 to 3, wherein an evaporation resistance of the composition is more than 1 s/cm, more than 2 s/cm or more than 3 s/cm, more than 5 s/cm, more than 9 s/cm, more than 10 s/cm, more than 13 s/cm, more than 15 s/cm, more than 20 s/cm, more than 25 s/cm or more than 30 s/cm.

Paragraph 5. The composition of any one of Paragraphs 1 to 4, wherein the carbon chain length of one or more FAHFAs or structural analogues thereof is C15-C100, C19-C72, C20-C55, C20-C50, C20-C40, C20-C35, C20-C25, C25-C45, C25-C40, C25-C35 or C25-C30, optionally the carbon chain length is C20, C21, C22, C23, C24, C25, C26, C27, C28, C29, C30, C31, C32, C33, C34, C35, C36, C37, C38, C39, C40, C41, C42, C43, C44, C45, C46, C47, C48, C49, C50, C51, C52, C53, C54 or C55.

Paragraph 6. The composition of any one of Paragraphs 1 to 5, wherein the FAHFA or a structural analogue thereof has the following formula (I)

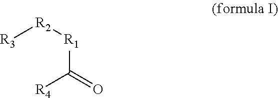

wherein
R₁ is a carbon atom, an oxygen atom or a nitrogen atom;
R₂ is a linear or branched C9-C50 alkyl, alkenyl or alkynyl chain, or a structural analogue thereof;
R₃ is a carboxyl, hydroxyl, amine, phosphate or silyl ether;
R₄ is a linear or branched C9-C50 alkyl, alkenyl or alkynyl chain, or a structural analogue thereof.

Paragraph 7. The composition of any one of the Paragraphs 1 to 6, wherein one or more FAHFAs are selected from the group comprising or consisting of O-Acyl-w-hydroxy fatty acids (OAHFAs).

Paragraph 8. The composition of any one of Paragraphs 1 to 7, wherein one or more FAHFAs and/or OAHFAs are selected from the group comprising or consisting of oleic acid based fatty acid esters, palmitoleic acid based fatty acid esters, myristoleic acid based fatty acid esters, lauric acid based fatty acid esters, paullinic acid based fatty acid esters, gondoic acid based fatty acid esters, erucic acid based fatty acid esters, nervonic acid based fatty acid esters, linoleic acid based fatty acid esters and linolenic acid based fatty acid esters; and/or one or more structural analogues of FAHFA are selected from the group comprising or consisting of oleic acid-based alcohols, palmitoleic acid-based alcohols, myristoleic acid-based alcohols, lauric acid-based alcohols, paullinic acid-based alcohols, gondoic acid-based alcohols, erucic acid-based alcohols, nervonic acid-based alcohols, linoleic acid based alcohols and linolenic acid based alcohols.

Paragraph 9. The composition of any one of Paragraphs 1 to 8, wherein one or more of the FAHFAs is selected from the group comprising or consisting of 12-OAHFA (12-(oleoyloxy)dodecanoic acid), 15-OAHFA (15-(oleoyloxy)pentadecanoic acid), 20-OAHFA (20-(oleoyloxy)eicosanoic acid), 22-OAHFA (22-(oleoyloxy)docosanoic acid), 20:1-OAHFA ((12Z)-20-(oleoyloxy)eicos-12-enoic acid) and 29:1-OAHFA ((21Z)-29-(oleoyloxy)nonacos-21-enoic acid).

Paragraph 10. The composition of any of one of Paragraphs 1 to 9, wherein the carbon chain length of one or more wax esters or structural analogs thereof is C15-C100, C19-C72, C20-C55, C20-C50, C20-C40, C20-C35, C20-C25, C25-C45, C25-C40, C25-C35 or C25-C30, optionally the carbon chain length is C20, C21, C22, C23, C24, C25, C26, C27, C28, C29, C30, C31, C32, C33, C34, C35, C36, C37, C38, C39, C40, C41, C42, C43, C44, C45, C46, C47, C48, C49, C50, C51, C52, C53, C54 or C55.

Paragraph 11. The composition of any of Paragraphs 1 to 10, wherein the wax ester or a structural analogue thereof has the following formula (II):

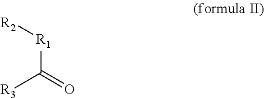

wherein
R₁ is a carbon atom, an oxygen atom or a nitrogen atom;
R₂ is a linear or branched C9-C50 alkyl, alkenyl or alkynyl chain, or a structural analogue thereof;
R₃ is a linear or branched C9-C50 alkyl, alkenyl or alkynyl chain, or a structural analogue thereof.

Paragraph 12. The composition of any one of Paragraphs 1 to 11, wherein one or more wax esters are selected from the group comprising or consisting of n-oleic acid based esters, iso-branched alkyl oleates, anteiso-branched alkyl oleates, palmitoleic acid based esters, iso-branched alkyl palmitoleates, anteiso-branched alkyl palmitoleates, myristoleic acid based esters, iso-branched alkyl myristoleates, anteiso-branched alkyl myristoleates, lauric acid based esters, iso-branched alkyl laurates, anteiso-based alkyl laurates, paullinic acid based esters, iso-branched alkyl paullinate, anteiso-branched alkyl paullinate, gondoic acid based esters, iso-branched alkyl gondoates, anteiso-based alkyl gondoates, erucic acid based esters, iso-branched alkyl eruciates, anteiso-branched alkyl eruciates, nervonic acid based esters, iso-branched alkyl nervonatess, anteiso-branched nervonate, linoleic acid based esters, iso-branched alkyl linoleates, anteiso-branched alkyl linoleates, linolenic acid based esters, iso-branched alkyl linolenates, and anteiso-branched alkyl linolenates.

Paragraph 13. The composition of any one of Paragraphs 1 to 12, wherein one or more wax esters are selected from the group comprising or consisting of palmityl oleate, stearyl oleate, arachidyl oleate (AO), behenyl oleate (BO), lignoceryl oleate, hexocosanyl oleate, 24-methylpentacosanyl oleate, palmityl palmitoleate, stearyl palmitoleate, arachidyl palmitoleate, behenyl palmitolate, lignoceryl palmitoleate, 24-methylpentacosanyl palmitoleate, palmityl linoleate, stearyl linoleate, arachidyl linoleate, behenyl palmitolate, lignoceryl linoleate, 24-methylpentacosanyl linoleate, palmityl linolenate, stearyl linolenate, arachidyl linolenate, behenyl palmitolate, lignoceryl linolenate, and 24-methylpentacosanyl linolenate.

Paragraph 14. The composition of any one of Paragraphs 1 to 13, wherein the one or more wax esters or structural analogues thereof are in the liquid state at the physiological conditions, and/or one or more wax esters or structural analogues thereof are in the solid state at the physiological conditions.

Paragraph 15. The composition of any one of Paragraphs 1 to 14, wherein the molar ratio of one or more FAHFAs and/or structural analogues thereof to one or more wax esters and/or or structural analogues thereof is 1:1 or less, about 1:1-1:100, or 1:2, 1:3, 1:4, 1:5, 1:6, 1:7, 1:8, 1:9 or 1:10, 1:20, 1:30, 1:40, 1:50, 1:60, 1:70, 1:80, 1:90.

Paragraph 16. The composition of any of Paragraphs 1 to 15, wherein the molar ratio of one or more FAHFAs and/or structural analogues thereof to one or more wax esters or structural analogues thereof is more than 1:1, about 100:1-1:1, about 3:2-5:1 or 90:1, 80:1, 70:1, 60:1, 50:1, 40:1, 30:1, 20:1, 10:1, 9:1, 8:1, 7:1, 6:1, 5:1, 4:1, 3:1, or 2:1.

Paragraph 17. The composition of any of Paragraphs 1 to 16, wherein one or more additives are selected from the group consisting of solvents, diluents, carriers, buffers, excipients, adjuvants, carrier media, antiseptics, fillers, stabilizers, thickening agents, emulsifiers, disintegrants, lubricants, and binders, and any combination thereof.

Paragraph 18. The composition of Paragraph 17, wherein one or more pharmaceutically acceptable excipients are ophthalmologically acceptable excipients selected from the group consisting of polyethylene glycol, propylene glycol, glycerin, polyvinyl alcohol, povidone, polysorbate 80, hydroxypropyl methylcellulose, carmellose, carbomer 980, sodium hyaluronate and dextran.

Paragraph 19. The composition of any one of Paragraphs 1 to 18, wherein the composition comprises at least 0.001 wt %, 0.005 wt %, 0.01 wt %, 0.05 wt %, 0.1 wt %, 0.5 wt %, 1 wt %, 2 wt %, 3 wt %, 4 wt %, 5 wt %, 6 wt %, 7 wt %, 8 wt %, 9 wt %, 10 wt %, 15 wt %, 20 wt %, 25 wt %, 30 wt %, 35 wt %, 40 wt %, 45 wt %, or 50 wt % FAHFAs, structural analogues thereof, and/or wax esters and/or structural analogues thereof.

Paragraph 20. The composition of any one of Paragraphs 1 to 19, wherein the composition is in a liquid, semisolid or solid form; the composition is in a form of a solution, emulsion, suspension, spray, powder, tablet, pellet, or capsule; or the composition is an oil-in-water emulsion.

Paragraph 21. The composition of any one of Paragraphs 1-20 for use as a medicament.

Paragraph 22. The composition of any one of Paragraphs 1-20 for use in the treatment of dry eye disease and/or Meibomian gland dysfunction, or for use in alleviation of eye discomfort.

Paragraph 23. A method of preparing a composition as defined in any of Paragraphs 1-20, wherein the method comprises combining or mixing one or more FAHFAs or structural analogues thereof and one or more wax esters or structural analogues thereof, and optionally one or more additives.

Paragraph 24. The method of Paragraph 23, wherein the method further comprises preparing or synthesizing the FAHFA or a structural analogue thereof before mixing it with the wax ester or a structural analogue thereof; and/or preparing or synthesizing the wax ester or a structural analogue thereof before mixing it with the FAHFA or a structural analogue thereof.

Paragraph 25. A non-therapeutic or therapeutic method of preventing evaporation of water, wherein the method comprises applying the composition of any one of Paragraphs 1-20 on a surface to be protected from evaporation or to a material to be protected from evaporation.

Paragraph 26. Use of the composition of any one of Paragraphs 1-20 for preventing evaporation of water.

Paragraph 27. A method of treating dry eye disease and/or Meibomian gland dysfunction, or alleviating eye discomfort, wherein the method comprises administering the composition of any one of Paragraphs 1-20 to the surface of an eye of a subject in need thereof.

### EXAMPLES

### 1. Materials and Methods

### 1.1 Synthesis of Lipids

All reagents were purchased from commercial sources. Dry solvents were purified by the VAC vacuum solvent purification system prior to use when dry solvents were needed. All reactions containing moisture- or air-sensitive reagents were carried out under an argon atmosphere. All reactions requiring heating were performed using an oil bath. Thin-layer chromatography (TLC) was performed on aluminium sheets pre-coated with silica gel 60 F254 (Merck). Flash chromatography was carried out using silica gel 40. Spots were visualized by UV followed by spraying with 1:4 H₂SO₄/MeOH-solution and heating. HRMS were recorded using a Bruker Micro Q-TOF with ESI (electrospray ionization) operated in positive mode. NMR spectra were recorded with a Bruker Avance III NMR spectrometer operating at 500.13 MHz (¹H) or 499.82 MHz (¹H), 125.68 MHz (¹³C) and 202.40 MHz (³¹P). All products were characterized by a combination of 1D (¹H, ¹³C and ³¹P) and 2D techniques (DQF-COSY, TOCSY, Ed-HSQC and HMBC) with pulse sequences provided by the instrument manufacturer. The probe temperature was kept at 25° C. unless otherwise stated. The chemical shifts are expressed on the 6 scale (in ppm) using TMS (tetramethylsilane) or residual chloroform as internal standards. Melting point analysis was performed using a Buchi B-545 melting point instrument (BOCHI Labortechnik AG) when possible.

### 1.1.1 Synthesis of FAHFAs

Here, the synthesis of FAHFAs will be exemplified by the synthesis of the following oleic acid derivatives: 20-OAHFA (20-(oleoyloxy)eicosanoic acid), 20:1-OAHFA ((12Z)-20-(oleoyloxy)eicos-12-enoic acid) and 29:1-OAHFA ((21Z)-29-(oleoyloxy)nonacos-21-enoic acid). A substantial library of structural analogues have likewise been prepared.

Other FAHFAs may be prepared by processes analogous to those described herein and/or by conventional synthetic procedures, in accordance with standard techniques, from commercially available starting materials or starting materials accessible by conventional synthetic procedures, using appropriate reagents and reaction conditions. In this respect, the skilled person may refer to inter alia “Comprehensive Organic Synthesis” by B. M. Trost and I. Fleming, Pergamon Press, 1991, “Comprehensive Organic Functional Group Transformations” by A. R. Katritzky, O. Meth-Cohn and C. W. Rees, Pergamon Press, 1995 and/or “Comprehensive Organic Transformations” by R. C. Larock, Wiley-VCH, 1999 and “March's Advanced Organic Chemistry: Reactions, Mechanisms, and Structure,”, by Michael B. Smith, John Wiley and Sons Ltd, Eighth Ed., 2020.

### Example 1. Synthesis of 20-(oleoyloxy)eicosanoic acid

20-hydroxyicosyl oleate. Oleic acid (1.2 equiv.), NaHSO₄·H₂O (3.5 mol %) and 1,20-Eicosanediol (0.12 g, 3.8 mmol, 1 equiv.) were added to a round bottomed flask. The stirred mixture was heated to 100° C. on an oil bath under vacuum. After 2.5 hours, the reaction was brought to rt, diluted with CHCl₃ (20 ml) and washed with saturated NaHCO₃ (15 ml). The organic layer was combined and washed with brine (15 ml), dried over Na₂SO₄, filtered and concentrated. The crude product was purified by column chromatography (EtOAc:hexane 1:3), concentrated and dried on the vacuum line to give the title compound as a white solid (0.10 g, 48% yield). Mp 59° C. 1H NMR (500.13 MHz; CDCl₃): δ 5.38-5.30 (m, 2H), 4.05 (t, 2H), 3.66-3.62 (m, 2H), 2.29 (t, 2H), 2.03-1.99 (m, 4H), 1.63-1.54 (m, 5H), 1.33-1.25 (m, 50H) and 0.88 (t, 3H) ppm. ¹³C NMR (125.68 MHz; CDCl₃): δ 174.2, 130.2, 129.9, 64.6, 63.3, 34.6, 33.0, 32.1-27.3, 26.1, 25.9, 25.2, 22.8, 22.2, 14.3 ppm. HRMS m/z: [M+H]⁺ Calcd. for C₃₈H₇₅O₃ 597.5716, found 597.5713.

20-(oleoyloxy)eicosanoic acid. A solution of 20-hydroxyeicosyl oleate (0.4026 g, 1 equiv.) in THF (15 ml), acetone (15 ml) and EtOAc (7.5 ml) under argon atmosphere was cooled to 0° C. and Jones reagent (0.770 ml, 2.2 equiv.) was added dropwise. The reaction mixture was stirred for 1 hour, quenched with 2-propanol (8 ml) and filtered through a pad of celite. The celite was then washed with Et₂O (80 ml) and the collected filtrate washed with brine (2×80 ml), dried over Na₂SO₄, filtered and concentrated. The crude product was purified using column chromatography (EtOAc:hexane:AcOH 5:95:0.1) and dried on the vacuum line to give the title compound as a white solid (0.345 g, 84% yield). Mp 62° C. 1H NMR (500.13 MHz; CDCl₃): δ 5.38-5.30 (m, 2H), 4.06 (t, 2H), 2.35 (t, 2H), 2.29 (t, 2H), 2.03-1.99 (m, 4H), 1.67-1.55 (m, 6H), 1.30-1.25 (m, 49H) and 0.88 (t, 3H) ppm. ¹³C NMR (125.68 MHz; CDCl₃): δ 177.1, 174.2, 130.2, 129.9, 64.6, 34.6, 33.7-27.3, 26.1, 25.2, 24.9, 22.8 and 14.3 ppm. HRMS m/z: [M+K]⁺ Calcd. for C₃₈H₇₂O₄K 631.5068, found 631.5005.

### Example 2. (12Z)-20-(oleoyloxy)eicos-12-enoic acid

12-bromo-1-dodecanol. To a solution containing 1,12-dodecanediol (2 g, 1 equiv.) in cyclohexane (26 ml) was added HBr (26 ml, 24 eq., 48% sol. in H₂O) and the biphasic system was refluxed for 18 h. The reaction mixture was then cooled to rt and the organic layer was separated. The aqueous phase was extracted with CH₂Cl₂ (5×25 ml). The combined organic phase was washed with sat. aq. NaHCO₃ solution (5×25 ml), brine (50 ml), dried over Na₂SO₄, filtered and concentrated. The crude product was purified by column chromatography (Hexane:EtOAc 7:3) and dried on the vacuum line to give the title compound as a white solid (2 g, 77% yield). ¹H NMR (500.13 MHz, CDCl₃): δ 3.64 (t, 2H), 3.41 (t, 2H), 1.85 (tt, 2H), 1.56 (tt, 2H), 1.42 (tt, 2H) and 1.38-1.22 (m, 14H) ppm. ¹³C NMR (125.68 MHz, CDCl₃): δ 63.2, 34.2, 33.0, 29.7-28.9, 28.3 and 25.9 ppm. HRMS (EI) m/z calculated for C₁₂H₂₅BrONa [M+Na]+287.0989, found 287.1001.

12-bromo-1-tert-butyldimethylsilyloxydodecane. To a solution containing 12-bromo-1-dodecanol (0.500 g, 1.2 equiv.) in dry CH₂Cl₂ (3 ml) was added imidazole (0.215 g, 2 equiv.). The reaction mixture was stirred under an argon atmosphere and after complete dissolution, TBDSMCI (0.238 g, 1 eq.) was added. The reaction mixture was stirred at rt for 18 h and then poured onto a cold sat. aq. NaHCO₃ solution (20 ml) and extracted with CH₂Cl₂ (3×20 ml). The combined organic phase was washed with H₂O (50 ml), dried over Na₂SO₄, filtered and concentrated. The crude oil was purified by column chromatography (Hexane:EtOAc 97:3) and dried on the vacuum line to give the title compound as a colorless thick oil (0.564 g, 94% yield). ¹H NMR (500.13 MHz, CDCl₃): δ 3.60 (t, 2H), 3.40 (t, 2H), 1.85 (tt, 2H), 1.50 (tt, 2H), 1.42 (tt, 2H), 1.35-1.22 (m, 14H), 0.89 (s, 9H) and 0.04 (s, 6H) ppm. ¹³C NMR (125.68 MHz, CDCl₃): δ 63.5, 34.1, 33.0, 29.8-28.9, 28.3, 26.1, 26.0, 18.5 and −5.1 ppm. HRMS (EI) m/z calculated for C₁₈H₃₉BrOSiNa [M+Na]⁺ 401.1854, found 401.1852.

1-tert-butyldimethylsilyloxydodecane, triphenylphosphonium bromide. A mixture containing 12-bromo-1-tert-butyldimethylsilyloxydodecane (2.0 g, 1 equiv.) and PPh₃ (1.39 g, 1 equiv.) was stirred under an argon atmosphere at 120° C. o/n and cooled to rt. The formation of the triphenylphosphonium bromide salt was confirmed by ³¹P NMR-analysis and the thick resinous product was used in the subsequent Wittig reaction as such. ³¹P NMR (202.4 MHz, CDCl₃): δ 24.4 ppm.

8-bromo-1-octanal. 8-bromo-1-octanol (1.01 g, 1 eq.) was dissolved in CH₂Cl₂ (80 ml) and PCC (1.563 g, 1.5 eq.) was added. The reaction mixture was stirred at rt for 3 h and then Et₂O (80 ml) was added, followed by filtration through celite to remove the remnants of PCC. The flask was washed with Et₂O (2×80 ml) and the combined filtrates were filtered through celite before concentration. H₂O (50 ml) and Et₂O (50 ml) were added to this residue. The light green organic phase was separated and the aqueous phase was extracted with CH₂Cl₂ (2×50 ml). The combined organic phase was washed with H₂O (100 ml), dried over Na₂SO₄, filtered and concentrated to give the title compound as an oil (84% yield). The crude product was used in the subsequent Wittig reaction as such. 1H NMR (500.13 MHz, CDCl₃): δ 9.74 (t, 1H), 3.38 (t, 2H), 2.41 (dt, 2H), 1.83 (tt, 2H), 1.61 (tt, 2H), 1.42 (tt, 2H) and 1.35-1.29 (m, 4H) ppm.

(12Z)-20-Bromo-1-tert-butyldimethylsilyloxyeicos-12-ene. A solution containing 1-tert-butyldimethylsilyloxydodecane, triphenylphosphonium bromide (3.164 g, 2 equiv.) in dry THF (30 ml) and HMPA (8.9 ml) under an argon atmosphere was cooled to −78° C. After 10 min., NaHMDS (8.2 ml, 0.6 M in toluene, 2 equiv.) was slowly added and the resulting mixture was stirred for 1 h. A solution of freshly prepared 8-bromo-1-octanal (0.536 g, 1.05 eq.) dissolved in dry THF (8 ml) was slowly added at −78° C. and the reaction mixture was allowed to warm to rt over 24 h before it was quenched with aq. phosphate buffer (freshly prepared, pH=7.2; 80 ml). Extraction with Et₂O (3×80 ml) was then performed and the combined organic phase was dried over Na₂SO₄, filtered and concentrated. The crude product was purified by column chromatography (Hexane:Et₃N 100:0.1→Hexane:EtOAc:Et₃N 399:1:0.1→98.7:1.3:0.1) and further dried on the vacuum line to give the title compound as a thick yellowish oil (0.427 g, 34% yield). 1H NMR (499.82 MHz, CDCl₃): δ 5.35 (dtt, 1H), 5.34 (dtt, 1H), 3.60 (t, 2H), 3.40 (t, 2H), 2.02 (ddt, 2H), 2.01 (ddt, 2H), 1.85 (tt, 2H), 1.50 (tt, 2H), 1.43 (tt, 2H), 1.38-1.22 (m, 22H), 0.89 (s, 9H) and 0.05 (s, 6H) ppm. ¹³C NMR (125.68 MHz, CDCl₃): δ 130.3, 129.9, 63.5, 34.2, 33.1, 33.0, 29.9-28.8, 28.3, 27.4-27.3, 26.2, 26.0, 18.6 and −5.1 ppm. HRMS (EI) m/z calculated for C₂₆H₅₃BrOSiNa [M+Na]⁺ 511.2949, found 511.2995.

(12Z)-20-acetoxy-1-tert-butyldimethylsilyloxyeicos-12-ene. To a solution containing 2 (0.265 g, 1 equiv.) in DMSO (12 ml) was added KOAc (0.266 g, 5 equiv.) and the suspension was stirred at rt o/n. After 24 h, additional KOAc (0.159 g, 3 equiv.) was added and the temperature was raised to 50° C. After 27 h, the reaction mixture was brought to rt and H₂O (25 ml) was added. Extraction with Et₂O (3×25 ml) was performed and the combined organic phase was washed with brine (30 ml), dried over Na₂SO₄, filtered and concentrated. The crude product was purified by column chromatography (Hexane:EtOAc:Et₃N 98:2:0.1→95.5:0.1) and dried on the vacuum line to give the title compound as a yellowish oil (0.169 g, 66% yield). ¹H NMR (500.13 MHz, CDCl₃): δ 5.35 (dtt, 1H), 5.34 (dtt, 1H), 4.05 (t, 2H), 3.59 (t, 2H), 2.04 (s, 3H), 2.01 (ddt, 2H), 2.00 (ddt, 2H), 1.62 (tt, 2H), 1.50 (tt, 2H), 1.39-1.22 (m, 24H), 0.89 (s, 9H) and 0.04 (s, 6H) ppm. ¹³C NMR (125.68 MHz, CDCl₃): δ 171.4, 130.2, 129.9, 64.8, 63.5, 33.1, 29.9-29.3, 28.8, 27.4-27.3, 26.2, 26.0, 21.2, 18.6 and −5.1 ppm. HRMS (EI) m/z calculated for C₂₈H₅₆O₃SiNa [M+Na]⁺ 491.3899, found 491.3879.

(12Z)-20-hydroxy-1-tert-butyldimethylsilyloxyeicos-12-ene. To a solution containing (12Z)-20-acetoxy-1-tert-butyldimethylsilyloxyeicos-12-ene (0.022 g, 1 equiv.) in MeOH (1 ml) and THF (0.5 ml) under argon atmosphere was added NaOMe (0.003 g, 1 eq.) and the resulting mixture was stirred at rt. After 22 h, the reaction was quenched by the addition of aq. HCl (10% sol., v/v; 3 drops) and H₂O (15 ml). The resulting mixture was extracted with Et₂O (3×15 ml) and the combined organic phase was dried over Na₂SO₄, filtered and concentrated. Drying under vacuum gave the title compound as a white solid (0.015 g, 78% yield). 1H NMR (500.13 MHz, CDCl₃): δ 5.34 (dtt, 1H), 5.34 (dtt, 1H), 3.64 (t, 2H), 3.59 (t, 2H), 2.01 (ddt, 2H), 2.00 (ddt, 2H), 1.57 (tt, 2H), 1.50 (tt, 2H), 1.40-1.22 (m, 24H), 0.89 (s, 9H) and 0.04 (s, 6H) ppm. ¹³C NMR (125.68 MHz, CDCl₃): δ 130.2, 129.9, 63.5, 63.3, 33.0, 32.9, 29.9-29.4, 27.4-27.3, 26.1, 26.0-25.9, 18.6 and −5.1 ppm. HRMS (EI) m/z calculated for C₂₆H₅₄O₂SiNa [M+Na]⁺ 449.3793, found 449.3832.

(12Z)-20-oleoyloxy-1-tert-butyldimethylsilyloxyeicos-12-ene. To a solution of 3 (0.040 g, 1 equiv.) in dry CH₂Cl₂ (2 ml) under argon atmosphere was added DMAP (0.012 g, 1 equiv.) and EDC·HCl (0.046 g, 2.5 eq.) and the resulting mixture was cooled to 0° C. on an ice-bath. Oleic acid (0.032 g dissolved in 0.5 ml dry CH₂Cl₂, 1.2 equiv.) was added and the reaction mixture was stirred at 0° C. for 10 min. and then at rt o/n. The reaction mixture was quenched after 20 h with H₂O (2 ml) and diluted with CH₂Cl₂ (10 ml). The organic phase was separated and the aqueous phase extracted with CH₂Cl₂ (2×10 ml). The combined organic phase was washed with H₂O (2×15 ml), dried over Na₂SO₄, filtered and concentrated. The crude product was purified by column chromatography (Hexane:EtOAc:Et₃N 95:5:0.1) and dried under vacuum to give the title compound as a white solid (0.061 g, 93% yield). ¹H NMR (500.13 MHz, CDCl₃): δ 5.35 (dtt, 1H), 5.35 (dtt, 1H), 5.34 (dtt, 1H), 5.34 (dtt, 1H), 4.05 (t, 2H), 3.60 (t, 2H), 2.29 (t, 2H), 2.06-1.96 (m, 8H), 1.62 (tt, 2H), 1.61 (tt, 2H), 1.50 (tt, 2H), 1.37-1.23 (m, 44H), 0.89 (s, 9H), 0.88 (t, 3H) and 0.05 (s, 6H) ppm. ¹³C NMR (125.68 MHz, CDCl₃): δ 174.1, 130.2-129.9, 64.5, 63.5, 34.5, 33.0, 32.1, 29.9-29.3, 28.8, 27.4-27.3, 26.1, 26.0, 25.2, 22.8, 18.6, 14.3 and −5.1 ppm. HRMS (EI) m/z calculated for C46H8303SiNa [M+Na]+713.6246, found 713.6214.

(12Z)-20-oleoyloxyeicos-12-enol. A solution containing (12Z)-20-oleoyloxy-1-tert-butyldimethylsilyloxyeicos-12-ene (0.031 g, 1 equiv.) in dry THF (0.5 ml) under argon atmosphere was cooled to 0° C. on an ice-bath and TBAF (0.140 ml, 1M in THF; 3 equiv.) was added. After 5 min., the ice-bath was removed and the reaction mixture stirred at rt for 1 h and then quenched with H₂O (2 ml) and extracted with EtOAc (3 ml). The organic phase was washed with H₂O (2×5 ml), the aqueous phase re-extracted with EtOAc (2×5 ml). The combined organic phase was dried over Na₂SO₄, filtered and concentrated. The crude product was purified by column chromatography (Hexane:EtOAc:Et₃N 7:3:0.1) and dried under vacuum to give the title compound a white solid (0.023 g, 92% yield). 1H NMR (500.13 MHz, CDCl₃): δ 5.35 (dtt, 1H), 5.35 (dtt, 1H), 5.34 (dtt, 1H), 5.34 (dtt, 1H), 4.05 (t, 2H), 3.64 (t, 2H), 2.29 (t, 2H), 2.06-1.96 (m, 8H), 1.62 (tt, 2H), 1.61 (tt, 2H), 1.56 (tt, 2H), 1.37-1.23 (m, 44H) and 0.88 (t, 3H) ppm. ¹³C NMR (125.68 MHz, CDCl₃): δ 174.2, 130.2-129.9, 64.5, 63.2, 34.5, 33.0, 32.1, 29.9-29.3, 28.8, 27.4-27.3, 26.0-25.9, 25.2, 22.8 and 14.3. HRMS (EI) m/z calculated for C₃₈H₇₂O₃Na [M+Na]⁺ 599.5381, found 599.5342.

(12Z)-20-oleoyloxyeicos-12-enoic acid. A solution containing 4 (0.026 g, 1 equiv.) in acetone (2 ml) and EtOAc (2 ml) was cooled to 0° C. on an ice-bath and Jones reagent (0.050 ml, 2.2 equiv.) was added. The resulting mixture was stirred at 0° C. for 45 min. H₂O (5 ml) was added and the reaction mixture was then extracted with Et₂O (3×15 ml). The combined organic phase was washed with brine (15 ml), dried over Na₂SO₄, filtered and concentrated. The crude product was purified by column chromatography (Hexane:EtOAc:AcOH 7:3:0.1) as eluent and dried on the vacuum line to give the title compound as a white solid (0.024 g, 89% yield). 1H NMR (500.13 MHz, CDCl₃): δ 5.35 (dtt, 1H), 5.35 (dtt, 1H), 5.34 (dtt, 1H), 5.34 (dtt, 1H), 4.05 (t, 2H), 2.34 (t, 2H), 2.29 (t, 2H), 2.06-1.96 (m, 8H), 1.63 (tt, 2H), 1.61 (tt, 2H), 1.61 (tt, 2H), 1.37-1.23 (m, 42H) and 0.88 (t, 3H) ppm. ¹³C NMR (125.68 MHz, CDCl₃): δ 179.6, 174.2, 130.1-129.9, 64.6, 34.5, 34.4, 32.0, 29.9-29.3, 28.8, 27.4-27.3, 26.1, 25.2, 24.9, 22.8 and 14.3. HRMS (EI) m/z calculated for C₃₈H₇₀O₄Na [M+Na]⁺ 613.5174, found 613.5175. Melting point: 29.5-30.7° C.

### Example 3. (21Z)-29-(oleoyloxy)nonacos-21-enoic acid

1,20-Eicosanediol. A solution containing eicosanedioic acid (0.5101 g, 1 equiv.) in 150 ml of THF was cooled to 0° C. on an ice bath. LAH (0.3419 g, 6.05 equiv.) was added portion wise and the mixture was brought to rt. The reaction mixture was heated to 85° C. and stirred for 18 h. The reaction was cooled down to 0° C. on an ice bath and satd. aqueous solution containing Rochelle's salt (40 ml) was added. The resulting mixture was stirred for 1 h and filtered through a celite pad. The filtrate was extracted with DCM (8×30 ml). The combined organic layers were dried over Na₂SO₄, filtered and concentrated to give the title compound as a white solid (0.405 g, 87% yield). 1H NMR (500.13 MHz, CDCl₃): δ 3.64 (t, 4H, 3=6.1 Hz), 1.56 (q, 4H) and 1.38-1.15 (m, 32H) ppm. ¹³C NMR (125.68 MHz, CDCl₃): δ 63.5, 33.2, 30.0-29.8 and 26.1 ppm. HRMS (EI) m/z calculated for C₂₀H₄₂O₂Na [M+Na]⁺ 337.3083, found 337.3152.

20-Bromoeicosan-1-ol. A mixture containing 1,20-eicosanediol (0.66 g, 1 equiv.), cyclohexane (24 ml) and HBr (9 ml, 48% in water) was heated at 82° C. for 5 h. The reaction was quenched with H₂O (20 ml) and the layers were separated. The aqueous layer was extracted with CH₂Cl₂ (4×20 ml). The combined organic layers were washed with a satd. aqueous solution of NaHCO₃ (30 ml) and H₂O (30 ml). The organic phase was separated, dried over Na₂SO₄, filtered and concentrated. The crude product was purified by flash chromatography (EtOAc:hexane) and dried on the vacuum line to give the title compound as a white solid (0.42 g, 52% yield). 1H NMR (500.13 MHz, CDCl₃): δ 3.64 (t, 2H, 3=6.1 Hz), 3.40 (t, 2H, 3=6.9 Hz), 1.85 (m, 2H), 1.56 (m, 2H) and 1.38-1.15 (m, 32H) ppm. ¹³C NMR (125.68 MHz, CDCl₃): δ 63.5, 34.4, 33.2, 31.9, 30.0-28.8, 28.5 and 26.1 ppm. HRMS (EI) m/z calculated for C₂₀H₄₁OBrNa [M+Na]⁺ 399.2233, found 399.2185.

20-Bromo-1-(tetrahydro-2H-pyran-2-yloxy)-eicosanol. To a solution containing 20-bromoeicosan-1-ol (0.23 g, 1 equiv.) in CH₂Cl₂ (20 ml) was added PPTS (0.03 g, 0.13 equiv.) and DHP (0.1 ml, 2.15 equiv.). The resulting mixture was stirred for 23 h at rt. The crude product was after concentration purified by flash chromatography (Hexane:EtOAc 9:1) to give the title compound as a white solid (0.27 g, 97% yield). 1H NMR (500.13 MHz, CDCl₃): δ 4.57 (t, 1H), 3.87 (m, 1H), 3.72 (m, 1H), 3.50 (m, 1H), 3.40 (t, 2H, J=6.89 Hz), 3.38 (m, 1H), 1.84 (m, 3H), 1.71 (m, 1H), 1.41 (m, 2H) 1.58 (m, 4H) and 1.38-1.15 (m, 32H) ppm. ¹³C NMR (125.68 MHz, CDCl₃): δ 98.8, 67.7, 62.3, 34.0, 32.8, 30.8, 29.8-28.2, 26.2, 25.5 and 19.7 ppm. HRMS (EI) m/z calculated for C₂₅H₄₉OBrNa [M+Na]⁺ 483.2814, found 483.2736.

1-O-tert-butyldimethylsilyl-non-8-yne. A solution containing non-8-yn-ol (0.5 g, 1 equiv.) and imidazole (0.567 g, 2.25 equiv.) in CH₂Cl₂ (20 ml) was cooled to 0° C. on an ice bath before adding TBDMSCI (1.0 g, 1.8 equiv.). The mixture was brought to rt and stirred for 18 h. The reaction was quenched by pouring the reaction mixture into 20 ml of an ice-cold satd. aqueous solution of NH₄Cl. The aqueous layer was separated and extracted with CH₂Cl₂ (4×30 ml). The crude product was purified by flash chromatography (EtOAc:hexane 9:1) and dried on the vacuum line to give the title compound as a colorless liquid (0.70 g, 78% yield). 1H NMR (500.13 MHz, CDCl₃): δ 3.58 (t, 2H, 3=6.6 Hz), 2.18 (dt, 2H), 1.93 (t, 1H, 3=2.7 Hz), 1.51 (m, 4H), 1.40 (m, 2H), 1.31 (m, 4H), 0.89 (s, 9H) and 0.05 (s, 6H) ppm. ¹³C NMR (125.68 MHz, CDCl₃): 84.7, 68.0, 63.2, 32.8, 28.9, 28.7, 28.4, 26.0, 25.7, 18.4 and −5.3 ppm. HRMS (EI) m/z calculated for C₁₅H₃₀OSi [M+Na]⁺ 277.1866, found 277.1924.

29-(Tetrahydro-2H-pyran-2-yloxy)nonacos-8-yn-1-ol. The solution of 1-O-tert-butyldimethylsilyl-non-8-yn-1-ol (0.236 g, 2.53 equiv.) in THF (3 ml) and HMPA (1 ml) was cooled to −78° C. using an EtOAc/N2 bath. To this solution, BuLi (0.25 ml, 2.39 equiv., 2.5 M in THF) was added dropwise and the temperature was allowed to rise to −40° C. and maintained for 2 h. The reaction mixture was then cooled back down to −78° C. and 20-bromo-1-(tetrahydro-2H-pyran-2-yloxy)-eicosanol (1 equiv.) in THF (3 ml) was added dropwise. The resulting mixture was brought to rt, TBAI (0.013 g, 1 mol %) was added and the temperature was raised to 80° C. The reaction was quenched after 20 h by pouring it onto a satd. aqueous solution of NH₄Cl (20 ml). The aqueous layer was separated and extracted with EtOAc (6×20 ml). The combined organic phase was dried over Na₂SO₄, filtered and concentrated. The crude product was dissolved in THF (10 ml) and the solution was cooled to 0° C. on an ice bath. TBAF (2.5 ml, 6.83 equiv., 1 M in THF) was added and the reaction mixture was brought to rt and stirred for 1 h. The reaction was quenched with H₂O (20 ml) and the aqueous layer was extracted with CH₂Cl₂ (5×20 ml). The combined organic phase was dried over Na₂SO₄, filtered and concentrated. The crude product was purified by flash chromatography (Hexane:EtOAc 9:1→4:1) and the fractions containing product were collected, concentrated and dried on the vacuum line to give the title compound as a white solid (0.099 g, 52% yield). ¹H NMR (500.13 MHz, CDCl₃): δ 4.57 (m, 1H), 3.87 (m, 1H), 3.72 (m, 1H), 3.64 (m, 1H), 3.50 (m, 1H), 3.38 (m, 1H), 2.13 (m, 4H), 1.83 (m, 1H), 1.71 (m, 1H) and 1.62-1.25 (m, 51H) ppm. ¹³C NMR (125.68 MHz, CDCl₃): 98.8, 67.7, 63.0, 62.3, 32.8, 30.8-28.8, 26.2, 25.6, 25.5, 19.7, 18.8 and 18.7 ppm. HRMS (EI) m/z calculated for C₃₄H₆₄O₃Na [M+Na]⁺ 543.4753, found 543.4648.

(8Z)-29-(Tetrahydro-2H-pyran-2-yloxy)nonacos-8-en-1-ol. 29-(Tetrahydro-2H-pyran-2-yloxy)nonacos-8-yn-1-ol (0.049 g, 1.0 equiv.) was dissolved in dry benzene (15 ml), and, Lindlar's catalyst (0.025 g) and quinoline (0.11 g, 9.0 equiv.) were added. The resulting mixture was placed inside a reactor and the air was replaced by a H₂-atmosphere (1 atm.). The reaction mixture was stirred for 1 h at rt. The hydrogen gas was removed and the reaction mixture was filtered through a pad of celite and concentrated. The crude product was purified by flash chromatography (hexane:EtOAc 4:1) and dried on the vacuum line to give the title compound as a white solid (0.044 g, 89% yield). 1H NMR (500.13 MHz, CDCl₃): δ 5.34 (m, 2H), 4.57 (m, 1H), 4.05 (t, 2H), 3.87 (m, 1H), 3.72 (m, 1H), 3.49 (m, 1H), 3.38 (m, 1H), 2.28 (t, 2H), 2.01 (m, 8H), 1.83 (m, 1H), 1.71 (m, 1H), 1.62-1.25 (51H) and 0.88 (t, 3H) ppm. HRMS (EI) m/z calculated for C₃₄H₆₆O₃Na [M+Na]⁺ 545.4910, found 545.4994.

(8Z)-29-(Tetrahydro-2H-pyran-2-yloxy)nonacos-8-en-1-yl oleate. (8Z)-29-(Tetrahydro-2H-pyran-2-yloxy)nonacos-8-en-1-ol (0.044 g, 1 equiv.) was dissolved in CH₂Cl₂ (2 ml) and DMAP (0.012 g, 1.16 equiv.) and EDC·HCl (0.037 g, 2.32 equiv.) was added to the reaction mixture with subsequent cooling on an ice bath. Oleic acid (0.055 g, 2.34 equiv.) in CH₂Cl₂ (2 ml) was added dropwise to the reaction mixture which was thereafter brought to rt and stirred for 22 h. The reaction was quenched by the addition of H₂O (20 ml) and the aqueous layer was isolated and extracted with DCM (7×10 ml). The combined organic phase was dried over Na₂SO₄, filtered and concentrated. The crude product was purified by flash chromatography (hexane:EtOAc 98:2→1:1) and dried on the vacuum line to give the title compound as a white solid (0.050 g, 76% yield). 1H NMR (500.13 MHz, CDCl₃): δ 5.34 (m, 4H), 4.57 (dt, 1H), 4.05 (t, 2H, 3=6.8 Hz), 3.87 (m, 1H), 3.78 (m, 1H), 3.49 (m, 1H), 3.38 (m, 1H), 2.28 (t, 2H, 3=7.5 Hz), 2.01 (m, 8H), 1.62-1.55 (6H), 1.38-1.20 (m, 62H) and 0.88 (t, 3H) ppm. ¹³C NMR (125.68 MHz, CDCl₃): δ 174.0, 130.0, 129.7, 98.8, 67.7, 64.4, 62.3, 34.4, 31.9, 30.8-27.2, 26.2, 25.9, 25.5, 25.0, 22.7, 19.7 and 14.1 ppm. HRMS (EI) m/z calculated for C₅₂H₉₈O₄Na [M+Na]⁺ 809.7139, found 809.7246.

(21Z)-29-(Oleoyloxy)nonacos-21-en-1-ol. (8Z)-29-(Tetrahydro-2H-pyran-2-yloxy)nonacos-8-en-1-yl oleate (0.050 g, 1 equiv.) was dissolved in MeOH:THF 3:1 (4 ml) and the resulting mixture was cooled to on an ice bath and CSA (0.003 g, 0.23 equiv.) was added. The reaction mixture was brought to rt and stirred for 18 h. The reaction was quenched by the addition of H₂O (20 ml) and the aqueous layer was extracted with CH₂Cl₂ (4×20 ml). The combined organic phase was dried over Na₂SO₄, filtered and concentrated. The crude product was purified by flash chromatography (hexane:EtOAc 9:1) and dried on the vacuum line to give the title compound as a white solid (0.032 g, 72% yield). 1H NMR (500.13 MHz, CDCl₃): δ 5.34 (m, 4H), 4.05 (t, 2H), 3.64 (q, 2H), 2.28 (2H), 2.05-1.96 (m, 8H), 1.62-1.55 (m, 6H), 1.38-1.20 (m, 62H) and 0.88 (t, 3H) ppm. ¹³C NMR (125.68 MHz, CDCl₃): δ 174.2, 130.2, 130.2, 130.0, 129.9, 64.6, 63.3, 34.6, 33.0, 32.1, 30.0-29.3, 28.9, 27.4, 27.4, 26.1, 25.9, 25.2 and 14.3 ppm. HRMS (EI) m/z calculated for C₄₈H₉₀O₃Na [M+H]⁺ 703.6968, found 703.7028. Melting point: 48.7-49.7° C.

(21Z)-29-(Oleoyloxy)nonacos-21-enoic acid. To a solution containing (21Z)-29-(oleoyloxy)nonacos-21-en-1-ol in Acetone:EtOAc 1:1 (4 ml) was added Jones reagent (0.05 ml, 2 M) and the resulting mixture was stirred for 1 h. The reaction was quenched with isopropanol (1 ml) and filtered through celite. After careful washing with EtOAc, the combined organic layers were washed with brine (2×30 ml), separated, dried over Na₂SO₄, filtered and concentrated. The crude product was purified by flash chromatography (hexane:EtOAc 9:1) and dried on the vacuum line to give the title compound as a white solid (0.02 g, 61% yield). 1H NMR (500.13 MHz, CDCl₃): δ 5.34 (m, 4H), 4.05 (t, 2H), 2.35 (t, 2H), 2.29 (t, 2H), 2.05-1.96 (m, 8H), 1.67-1.57 (m, 6H), 1.38-1.20 (m, 62H) and 0.88 (t, 3H) ppm. ¹³C NMR (125.68 MHz, CDCl₃): δ 177.4, 174.4, 130.2, 130.0, 129.9, 60.6, 34.6, 33.7, 32.1, 30.0-29.3, 28.8, 27.4, 27.3, 26.1, 25.2, 24.9, 22.9 and 14.3 ppm. HRMS (EI) m/z calculated for C₄₇H₈₈O₄Na [M+Na]⁺ 739.6581 found 739.6803. Melting point: 53.0-54.0° C.

### 18-(oleoyloxy)stearic acid (18:0/18:1-OAHFA; 18-OAHFA)

Oleic acid (1.29 g, 4.6 mmol, 0.93 equiv.), NaHSO₄·H₂O (0.024 g, 3.5 mol %) and 1,18-Octadecanediol (1.40 g, 4.9 mmol, 1 equiv.) were added to a round bottomed flask (100 ml). The stirred mixture was heated to 100° C. on an oil bath under vacuum. After 2 hours, the reaction was brought to rt, diluted with CHCl₃ (50 ml) and washed with saturated NaHCO₃ (2×50 ml). The aqueous phase was re-extracted with CHCl₃ (2×50 ml) and the organic layers were combined and washed with brine (80 ml), dried over Na₂SO₄, filtered and concentrated. The crude product was purified by column chromatography (Hexane:EtOAc 19:1→4:1), concentrated and dried on the vacuum line to give 18-hydroxyoctadecyl oleate as a white solid (1.21 g, 45% yield). Mp 51.9-53.0° C. ¹H NMR (499.82 MHz, CDCl₃, 25° C.): δ 5.39-5.30 (m, 2H), 4.05 (t, 2H), 3.66-3.60 (m, 2H), 2.28 (t, 2H), 2.04-1.97 (m, 4H), 1.66-1.52 (m, 6H), 1.38-1.21 (m, 48H) and 0.88 (t, 3H) ppm. ¹³C NMR (125.68 MHz; CDCl₃): δ 174.1, 130.1, 129.9, 64.6, 63.3, 34.6, 33.0, 32.1, 29.9-29.3, 28.8, 27.4, 27.3, 26.1, 25.9, 25.2, 22.8, 14.3 ppm.

A solution of 18-hydroxyoctadecyl oleate (0.7426 g, 1.4 mmol, 1 equiv.) in THF (15 ml), acetone (15 ml) and EtOAc (7.5 ml) under argon atmosphere was cooled to 0° C. and Jones reagent (1.51 ml, 3.0 mmol, 2.3 equiv.) was added dropwise. The reaction mixture was stirred for 1.5 hour, quenched with 2-propanol (10 ml) and filtered through a pad of celite. The celite was then washed with Et₂O (100 ml) and the collected filtrate washed with brine (2×100 ml), dried over Na₂SO₄, filtered and concentrated. The crude product was purified using column chromatography (Hexane:EtOAc:AcOH 19:1:0.01→7:3:0.01) and dried on the vacuum line to give the title compound as a white solid (0.582 g, 78% yield). Mp 55.8-56.7° C. ¹H NMR (499.82 MHz, CDCl₃, 25° C.): δ 5.39-5.29 (m, 2H), 4.05 (t, 2H), 2.34 (t, 2H), 2.29 (t, 2H), 2.06-1.97 (m, 4H), 1.67-1.56 (m, 6H), 1.37-1.21 (m, 46H) and 0.88 (t, 3H) ppm. ¹³C NMR (125.68 MHz; CDCl₃): δ 179.5, 174.2, 130.1, 130.0, 64.6, 34.6, 34.1, 32.1, 29.9-29.2, 28.8, 27.4, 27.3, 26.1, 25.2, 24.8, 22.8 and 14.3 ppm. HRMS m/z calculated for C₃₆H₆₈O₄Na [M+Na]⁺ 587.5015, found 587.5030.

### Further FAHFAs

The following FAHFAs have also been prepared following analogous synthetic processes to those described for 20-(oleoyloxy)eicosanoic acid (Example 1).
12-(linoleoyloxy)dodecanoic acid,
20-(linoleoyloxy)eicosanoic acid,
12-(palmitoleoyloxy)dodecanoic acid,
20-(palmitoleoyloxy)eicosanoic acid,
12-(palmitoyloxy)dodecanoic acid,
20-(palmitoyloxy)eicosanoic acid
12-(stearoyloxy)dodecanoic acid and
20-(stearoyloxy)eicosanoic acid.

The characterization data for the above FAHFAs are provided in the table below.

TABLE 1
Characterization data

| Compound | Data |
|---|---|
| 12- | Mp 36.8° C.; |
| (palmitoleoyloxy)dodecanoic | ¹H NMR (499.82 MHz; CDCl3; 25° C.) δ 5.39-5.29 (m, |
| acid | 2H), 4.05 (t, 2H), 2.35 (t, 2H), 2.29 (t, 2H), 2.06- |
| | 1.95 (m, 4H), 1.69-1.54 (m, 6H), 1.39-1.19 (m, 30H) |
| | and 0.88 (t, 3H) pp; |
| | ¹³C NMR (125.68 MHz; CDCl3; 25° C.) δ 179.2, 174.2, |
| | 130.1, 129.9, 64.6, 34.6, 34.0, 31.9, 29.7-29.0, |
| | 28.8, 27.4, 27.3, 26.1, 25.2, 24.8, 22.8 and 14.2 |
| | ppm; |
| | HRMS m/z calculated for C28H52O4Na [M + Na]+ |
| | 475.3758, found 475.3681. |
| 12- | Mp 66.7° C.; |
| (palmitoyloxy)dodecanoic | ¹H NMR (499.82 MHz; CDCl3; 25° C.) 4.05 (t, 2H), |
| acid | 2.35 (t, 2H), 2.29 (t, 2H), 1.68-1.55 (m, 6H), 1.41- |
| | 1.16 (m, 38H) and 0.88 (t, 3H) ppm; |
| | ¹³C NMR (125.68 MHz; CDCl3; 25° C.) δ 179.2, 174.2, |
| | 64.6, 34.6, 34.0, 32.1, 29.8-29.2, 28.8, 26.1, 25.2, |
| | 24.8, 22.8 and 14.3 ppm; |
| | HRMS m/z calculated for C28H54O4Na [M + Na]+ |
| | 477.3914, found 477.3868. |
| 12- | Mp 71.2° C.; |
| (stearoyloxy)dodecanoic | ¹H NMR (499.82 MHz; CDCl3; 25° C.) 4.05 (t, 2H), |
| acid. | 2.35 (t, 2H), 2.29 (t, 2H), 1.68-1.53 (m, 6H), 1.39- |
| | 1.17 (m, 42H) and 0.88 (t, 3H) ppm; |
| | ¹³C NMR (125.68 MHz; CDCl3; 25° C.) δ 178.9, 174.2, |
| | 64.5, 34.6, 34.0, 32.1, 29.8-29.2, 28.8, 26.1, 25.2, |
| | 24.8, 22.8 and 14.3 ppm; |
| | HRMS m/z calculated for C30H58O4Na [M + Na]+ |
| | 505.4227, found 505.4251. |
| 12- | ¹H NMR (499.82 MHz; CDCl3; 25° C.) δ 5.42-5.28 (m, |
| (linoleoyloxy)dodecanoic | 4H), 4.05 (t, 2H), 2.77 (m, 2H), 2.35 (t, 2H), 2.29 (t, |
| acid. | 2H), 2.08-1.99 (m, 4H), 1.69-1.53 (m, 6H), 1.39- |
| | 1.16 (m, 28H) and 0.88 (t, 3H) ppm; |
| | ¹³C NMR (125.68 MHz; CDCl3; 25° C.) δ 178.9, 174.2, |
| | 130.4, 130.2, 128.2, 128.1, 64.6, 34.6, 33.9, 31.7, |
| | 29.8-29.2, 28.8, 27.4, 27.3, 26.1, 26.1, 25.8, 25.2, |
| | 24.8, 22.7 and 14.2 ppm; |
| | HRMS m/z calculated for C30H54O4Na [M + Na]+ |
| | 501.3914, found 501.3931. |
| 20- | Mp 61.0° C.; |
| (palmitoleoyloxy)eicosanoic | ¹H NMR (499.82 MHz; CDCl3; 25° C.) δ 5.39-5.30 (m, |
| acid. | 2H), 4.05 (t, 2H), 2.35 (t, 2H), 2.29 (t, 2H), 2.05- |
| | 1.96 (m, 4H), 1.68-1.54 (m, 6H), 1.38-1.20 (m, 46H) |
| | and 0.88 (t, 3H) ppm; |
| | ¹³C NMR (125.68 MHz; CDCl3; 25° C.) δ 178.6, 174.2, |
| | 130.1, 129.9, 64.6, 34.6, 33.9, 31.9, 29.9-29.1, 28.8, |
| | 27.4, 27.3, 26.1, 25.2, 24.9, 22.8 and 14.2 ppm; |
| | HRMS m/z calculated for C36H68O4Na [M + Na]+ |
| | 587.5010, found 587.5051. |
| 20-(palmitoyloxy)eicosanoic | Mp 76.8° C.; |
| acid. | ¹H NMR (499.82 MHz; CDCl3; 25° C.) 4.05 (t, 2H), |
| | 2.35 (t, 2H), 2.29 (t, 2H), 1.69-1.51 (m, 6H), 1.49- |
| | 1.14 (m, 54H) and 0.88 (t, 3H) ppm; |
| | ¹³C NMR (125.68 MHz; CDCl3; 25° C.) δ 177.5, 174.2, |
| | 64.6, 34.6, 33.7, 32.1, 29.8-29.2, 28.8, 26.1, 25.2, |
| | 24.9, 22.8 and 14.3 ppm; |
| | HRMS m/z calculated for C36H70O4Na [M + Na]+ |
| | 589.5166, found 589.5145. |
| 20-(linoleoyloxy)eicosanoic | Mp 53.6 C; |
| acid. | ¹H NMR (499.82 MHz; CDCl3; 25° C.) δ 5.42-5.28 (m, |
| | 4H), 4.05 (t, 2H), 2.77 (m, 2H), 2.35 (t, 2H), 2.29 (t, |
| | 2H), 2.08-1.99 (m, 4H), 1.67-1.55 (m, 6H), 1.40- |
| | 1.19 (m, 44H) and 0.89 (t, 3H) ppm; |
| | ¹³C NMR (125.68 MHz; CDCl3; 25° C.) δ 178.6, 174.2, |
| | 130.4, 130.2, 128.2, 128.1, 64.6, 34.6, 33.9, 31.7, |
| | 29.8-29.2, 28.8, 27.4, 26.1, 25.8, 25.2, 24.9, 22.7 |
| | and 14.2 ppm; |
| | HRMS calculated for C38H70O4Na [M + Na]+ |
| | 613.5166, found 613.5164. |
| 20-(stearoyloxy)eicosanoic | HRMS calculated for C38H74O4Na [M + Na]+ |
| acid. | 617.5479, found 617.5455. |

### 1.1.2 Synthesis of Wax Esters

A number of wax esters are commercially available such as behenyl oleate, arachidyl oleate etc. and can be used in the present invention. Here, the synthesis of non-commercially available wax esters will be exemplified by the synthesis of the following oleic acid derivatives: hexocosanyl oleate and 24-methylpentacosanyl oleate. A substantial library of structural analogues have likewise been prepared.

Other wax esters may be prepared by processes analogous to those described herein and/or by conventional synthetic procedures, in accordance with standard techniques, from available starting materials or starting materials accessible by conventional synthetic procedures, using appropriate reagents and reaction conditions. In particular, such compounds may be prepared by Fischer esterification of the corresponding carboxylic acids and alcohols and/or by reaction of the corresponding acid chlorides or acid anhydrides with the corresponding alcohol under standard reaction conditions. Such carboxylic acids, acid chlorides, acid anhydrides and alcohols may be commercially available or may be prepared according to conventional synthetic procedures as known to the skilled person. In this respect, the skilled person may refer to inter alia “Comprehensive Organic Synthesis” by B. M. Trost and I. Fleming, Pergamon Press, 1991, “Comprehensive Organic Functional Group Transformations” by A. R. Katritzky, O. Meth-Cohn and C. W. Rees, Pergamon Press, 1995 and/or “Comprehensive Organic Transformations” by R. C. Larock, Wiley-VCH, 1999 and “March's Advanced Organic Chemistry: Reactions, Mechanisms, and Structure,”, by Michael B. Smith, John Wiley and Sons Ltd, Eighth Ed., 2020.

### Example 4. Hexacosyl Oleate

Hexacosyl oleate. To a solution of commercially available 1-hexacosanol (0.03 g, 1 equiv.) in dry CH₂Cl₂ (2.5 ml) and pyridine (1 ml) under argon atmosphere was added DMAP (0.01 g, 1 equiv.) and EDC·HCl (0.0380 g, 2.5 equiv.). Oleic acid (0.027 g in 0.5 ml dry CH₂Cl₂; 1.2 equiv.) was added dropwise. The resulting mixture was stirred o/n at rt. The reaction mixture was quenched after 18.5 h with H₂O (5 ml) and diluted with CH₂Cl₂ (10 ml). The organic phase was separated and the aqueous layer extracted with CH₂Cl₂ (3×10 ml). The combined organic layers were washed with H₂O (2×20 ml), dried over Na₂SO₄, filtered and concentrated. The crude product was purified by column chromatography (Hexane:EtOAc 95:5) and dried on the vacuum line to give the title compound as a white waxy solid (0.047 g, 93% yield). Mp 47.0-49.0° C. 1H NMR (500.13 MHz, CDCl₃): δ 5.35 (dtt, 1H), 5.33 (dtt, 1H), 4.05 (t, 2H), 2.29 (t, 2H), 2.01 (ddt, 2H), 2.00 (ddt, 2H), 1.62 (tt, 2H), 1.60 (tt, 2H), 1.38-1.19 (m, 66H), 0.88 (t, 3H) and 0.88 (t, 3H) ppm. ¹³C NMR (125.68 MHz, CDCl₃): δ ¹³C NMR (125.68 MHz, CDCl₃): δ 174.1, 130.1, 129.9, 64.5, 39.2, 34.6, 32.1-32.0, 29.9-29.7, 29.5-29.4, 29.3-29.2, 28.8, 27.4-27.3, 26.1, 25.2, 22.8 and 14.3 ppm. HRMS (EI) m/z calculated for C₄₄H₈₆O₂Na [M+Na]⁺ 669.6528, found 669.6693.

### Example 5. 24-methylpentacosyl oleate

1-(2-tetrahydropyranyloxy)-24-methylpentacos-20-yne. A solution containing 4-methyl-1-pentyne (0.65 ml, 5 equiv.) in dry THF (4 ml) and HMPA (1.5 ml) under an argon atmosphere was cooled to −78° C. n-BuLi (2.2 ml, 2.5 M in hexane, 5 equiv.) was slowly added and the resulting mixture was stirred at −40° C. for 2 h. The temperature was then again lowered to −78° C. and a solution of 20-bromo-1-(2-tetrahydropyranyloxy)eicosane (0.500 g, 1 equiv.) dissolved in dry THF (5 ml) was slowly added and the reaction mixture was allowed to warm to rt. TBAI (0.040 g, 0.1 eq.) was added and the reaction mixture was stirred 10 min. at rt. and then refluxed at 80° C. o/n. The reaction mixture was then quenched with a satd. solution of NH₄Cl (20 ml) and extracted with Et₂O (5×20 ml). The combined organic phase was washed with H₂O (2×20 ml), dried over Na₂SO₄, filtered and concentrated under reduced pressure. The crude product was purified by column chromatography (Hexane:EtOAc 98:2→90:10) and dried on the vacuum line to give the title compound as a yellowish solid (0.445 g, 89% yield). ¹H NMR (500.13 MHz, CDCl₃): δ 4.57 (t, 1H), 3.92-3.83 (m, 1H), 3.77-3.68 (m, 1H), 3.55-3.46 (m, 1H), 3.42-3.33 (m, 1H), 2.19-2.11 (m, 2H), 2.07-2.01 (m, 2H), 1.88-1.67 (m, 3H), 1.64-1.43 (m, 8H), 1.42-1.33 (m, 4H), 1.32-1.17 (m, 28H), 0.96 (d, 3H) and 0.96 (d, 3H) ppm. ¹³C NMR (125.68 MHz, CDCl₃): δ 98.9, 81.3, 79.2, 67.9, 62.5, 30.9, 29.9-29.0, 28.5, 28.2, 26.4, 25.7, 22.1, 19.9 and 18.9 ppm. HRMS (EI) m/z calculated for C₃₁H₅₈O₂Na [M+Na]⁺ 485.4337, found 485.4325.

1-hydroxy-24-methylpentacos-20-yne. To a solution of 1-(2-tetrahydropyranyloxy)-24-methylpentacos-20-yne (0.153 g, 1 equiv.) in dry THF/MeOH (6 ml, 1:3 ratio) under argon atmosphere was added CSA (0.008 g, 0.1 eq.) and the resulting reaction mixture was stirred at rt. o/n and then concentrated under reduced pressure. The crude product was purified by column chromatography (Hexane:EtOAc 100:0→4:1) and dried on the vacuum line to give the title compound as a white solid (0.118 g, 95% yield). ¹H NMR (500.13 MHz, CDCl₃): δ 3.64 (t, 2H), 2.17-2.12 (m, 2H), 2.06-2.00 (m, 2H), 1.81-1.70 (m, 1H), 1.57 (tt, 2H), 1.48 (tt, 2H), 1.41-1.20 (m, 32H), 0.96 (d, 3H) and 0.96 (d, 3H) ppm. ¹³C NMR (125.68 MHz, CDCl₃): δ 81.3, 79.2, 63.3, 32.9, 29.8-29.0, 28.5, 28.2, 25.9, 22.1 and 18.9 ppm. HRMS (EI) m/z calculated for C₂₆H₅₀ONa [M+Na]⁺ 401.3762, found 401.3760.

1-hydroxy-24-methylpentacosane. To a solution of 1-hydroxy-24-methylpentacos-20-yne (0.110 g, 1.0 equiv.) in dry EtOAc (15 ml) Pd/C (10% Pd, 0.220 g, 2 mass equiv.) was added. The reaction mixture was stirred in an autoclave under H₂-pressure (6 bar) for 4 h and filtered through celite. The celite was washed with EtOAc (20 ml) and the filtrate concentrated under reduced pressure. The crude product was purified (Hexane:EtOAc 4:1) and dried on the vacuum line to give the title compound as a white solid (0.093 g, 84% yield). 1H NMR (500.13 MHz, CDCl₃): δ 3.64 (t, 2H), 1.56 (tt, 2H), 1.53-1.47 (m, 1H), 1.39-1.18 (m, 40H), 1.15 (q, 2H), 0.86 (d, 3H) and 0.86 (d, 3H) ppm. ¹³C NMR (125.68 MHz, CDCl₃): δ 63.3, 39.2, 32.9, 30.1, 29.9-29.6, 28.1, 27.6, 25.9 and 22.8 ppm. HRMS (EI) m/z calculated for C₂₆H₅₄ONa [M+Na]⁺ 405.4075, found 405.4028.

24-methylpentacosyl oleate. To a solution of 1-hydroxy-24-methylpentacosane (0.035 g, 1 equiv.) in dry CH₂Cl₂ (2.5 ml) and dry pyridine (1 ml) under argon atmosphere was added DMAP (0.012 g, 1 equiv.) and EDC·HCl (0.044 g, 2.5 equiv.). Oleic acid (0.031 g in 0.5 ml dry CH₂Cl₂; 1.2 equiv.) was added dropwise. The resulting mixture at rt. o/n. The reaction mixture was quenched after 18 h with H₂O (10 ml) and diluted with CH₂Cl₂ (10 ml). The organic phase was separated and the aqueous layer extracted with CH₂Cl₂ (4×10 ml). The combined organic layers were washed with H₂O (2×15 ml), dried over Na₂SO₄, filtered and concentrated. The crude product was purified by column chromatography (Hexane:EtOAc 95:5) and dried on the vacuum line to give the title compound as a white waxy solid (0.057 g, 96% yield). Mp 36.0-37.5° C. 1H NMR (500.13 MHz, CDCl₃): δ 5.36 (dtt, 1H), 5.34 (dtt, 1H), 4.05 (t, 2H), 2.29 (t, 2H), 2.01 (ddt, 2H), 2.00 (ddt, 2H), 1.62 (tt, 2H), 1.60 (tt, 2H), 1.56-1.47 (m, 1H), 1.38-1.19 (m, 60H), 1.15 (q, 2H), 0.88 (t, 3H, overlapping), 0.86 (d, 3H, overlapping) and 0.86 (d, 3H, overlapping) ppm. ¹³C NMR (125.68 MHz, CDCl₃): δ 174.1, 130.1, 129.9, 64.6, 39.2, 34.6, 32.1, 30.1, 29.9-29.3, 28.8, 28.1, 27.6, 27.4-27.3, 26.1, 25.2, 22.8 and 14.3 ppm. HRMS (EI) m/z calculated for C₄₄H₈₇O₂ [M+H]⁺ 647.6708, found 647.6725.

### 1.2 Characterization of the Surface Organization and Evaporation Resistance of Lipid Compositions Comprising a FAHFA and a Wax Ester

### 1.2.1 Materials and Preparation of Lipid Compositions

The lipid species were either synthesized as described above or obtained from commercial sources (e.g. Nu-Check-Prep, Elysian, MN) and studied on their own at first. A wide range of lipid mixtures were prepared by weighing selected compounds at specific ratios and further dilution in chloroform to obtain selected concentrations. A 2 mM concentration of the corresponding mixtures was used in the surface potential and pressure measurements and a 5 mM concentration in the evaporation resistance measurements discussed herein.

### Example 6a. Mixtures Containing Arachidyl Oleate (AO) or Behenyl Oleate (BO) and 20-(oleoyloxy)eicosanoic acid (20-OAHFA)

A number of mixtures consisting of 20-OAHFA and either AO or BO were prepared by the methods described above. Herein we will focus the discussion on four different 20-OAHFA:AO mixtures with molar ratios of: 1:1, 1:3, 1:6, 1:9; and, six different 20-OAHFA:BO mixtures with molar ratios of: 3:1, 2:1, 1:1, 1:2 1:3, 1:9.

### Example 6b Mixtures of 18-(oleoyloxy)stearic acid (18:0/18:1-OAHFA) and Behenyl Oleate (BO), 18:0/18:1-OAHFA and Behenyl Behenoate (BB), 18:0/18:1-OAHFA and Arachidyl Laurate (AL) and (29:1/18:1-OAHFA) and BO

1:1 mixtures of 18-(oleoyloxy)stearic acid (18:0/18:1-OAHFA) and behenyl oleate (BO), 18:0/18:1-OAHFA and behenyl behenoate (BB), 18:0/18:1-OAHFA and arachidyl laurate (AL) and (29:1/18:1-OAHFA) and BO were prepared using the method described above and a 5 mM solution of each mixture in chloroform was used in the evaporation resistance measurement described herein.

### 1.2.2 Preparation of the Formulations

The formulations were prepared by mixing FAHFAs or structural analogues thereof and wax esters or structural analogues thereof with appropriate additives, such as emulsifiers to create a stable emulsion.

### Example 7. Formulations Containing 20-(oleoyloxy)eicosanoic acid (20-OAHFA) and Behenyl Oleate (BO)

Formulations containing 0.25 wt % of 20-OAHFA, 0.75 wt % of BO, 0.8% w/v Miglyol 812, 2% w/v Tween 20, 0.5% w/v Kolliphor® EL, 0.7% w/v Span 80, 2.2% w/v glycerin, and 98.2% w/v ultrapure water were prepared by mixing at 76° C.

### 1.2.3 Surface Pressure, Brewster Angle Microscopy, and Surface Potential

The lipid mixtures dissolved in chloroform or the formulations were spread onto the air-buffer interface of either a KSV minitrough (Helsinki, Finland) or a KSV large trough which was filled with PBS buffer. Chloroform was allowed to evaporate for 3 min before starting measurements. The surface pressure was measured using a Wilhelmy plate, the surface potential was measured using a KSV surface potential sensor (Espoo, Finland) and Brewster angle microscopy images were captured using a KSV NIMA microBAM (Espoo, Finland) instrument. The films were compressed at a constant rate of 5 or 10 mm/min and the subphase temperature was kept at 35±1° C. The measurements were performed in an acrylic box under an ozone-free atmosphere to prevent undesired oxidation. An ozone-free atmosphere was generated by passing dry air through ODS-3P ozone destruct unit (Ozone solutions, Hull, Iowa) at a rate of 76 l/min within the enclosure.

### 1.2.4 Evaporation Resistance

Evaporation resistance is a property of the lipid film residing on top or above an aqueous surface, independent of the measurement method and conditions of the measurement. It is defined as r=Δc/J, where Δc is the water vapor concentration difference driving evaporation and J is the evaporative flux from the underlying aqueous phase defined as J=(dn/dt)/A, where n is the amount of water evaporating, t is the time, and A is the area of the surface. Evaporation resistance of the lipid film can be determined, for example, by measuring the evaporative flux from the aqueous surface without the lipid layer, J_{w}, and with a lipid film present, J_{f}. The total evaporation resistance without the lipid film present, r_{w}, consists of various components, such as the diffusion and convection in the air layer overlying the aqueous surface, which depend on the measurement setup. The total evaporation resistance with the lipid film present, r_{f}, contains these same components with an additional resistance term, rₘ, caused by the lipid film: r_{f}=r_{w}+rₘ. The lipid film evaporation resistance can be expressed as a function of measured quantities as rₘ=Δc(1/J_{f}−1/J_{w}).

In this example, the evaporation resistance was determined according to the method originally developed by Langmuir et al. (Langmuir, I. and Schaefer, V., J. 1943, J. Franklin Inst., Vol. 235, 119-162) although with certain modifications (as set out in Bland et al., Langmuir, 2019, 35, 3545-3552 (Supp. Info.).

The measurements were conducted by compressing the film to a specific (mean molecular area) Mma, ranging between 2-40 Å²/molecule, and placing a tailored desiccant cartridge, with a water-permeable membrane, approx. 2 mm above the aqueous surface. The commercial silica gel containing desiccant cartridges (SP Industries, Warminster, PA) were modified by replacing the membrane with a Millipore Immobilon-P PVDF membrane (450 nm pore size, Bedford, MA). The desiccant cartridge was fixed in position for 5 minutes and the mass of the absorbed water was determined by gravimetric techniques. A second background measurement was conducted in parallel inside the enclosed acrylic box in order to account for the water absorbed from the dry air inside the enclosure. The results are therefore an accurate representation of the water evaporation from the aqueous surface.

### 2. Results

### 2.1 Chemical Synthesis

Throughout the synthesis, the products were purified by chromatographic techniques and characterized in detail by a wide range of NMR-spectroscopic techniques (e.g. ¹H, ¹³C, ³¹P, DQF-COSY, Ed-HSQC, TOCSY and HMBC) and high-resolution mass spectrometry thus ensuring their structural identity and purity. Our thorough structural characterization flow has recently been highlighted in the Journal of Organic Chemistry (Viitaja, T. et. al. 2021, J. Org. Chem. 86, 4965-4976) and therefore these aspects will not be further commented on in this document.

Three separate routes for the synthesis of FAHFA analogues of different complexity have been either developed and/or modified from literature protocols (see for example; Bland et. al. Langmuir 2019, 35, 9, 3545-3552; Viitaja et. al. J. Org. Chem. 2021, 86, 4965-4976; Hancock et. al. J. Lipid Res. 2018, 59, 1510-1518 and references therein etc.). Oleic acid derivatives are described throughout this section since this is the most abundant acyl chain in TFLL FAHFAs, however, derivatives with different acyl chains, including linoleic acid, palmitoleic acid, palmitic acid and stearic acid, have likewise been prepared.

The first route is presented in Scheme 1 and has been developed for the synthesis of non-complex FAHFA analogues. By this route, the synthesis of FAHFA-analogues can be completed in as little as two synthetic steps. The chain length and saturation degree of the starting material can be tailored and the esterification can be conducted with a wide range of carboxylic acids. The route will here be exemplified by the synthesis of 20-(oleoyloxy)eicosanoic acid although a number of structural analogues have likewise been prepared.

1,20-Eicosanediol was subjected to a Fisher esterification with oleic acid (1.2 equiv.) employing a catalytic amount of sodium bisulfate as a catalyst. Neat reaction conditions were employed and the reaction was performed at elevated temperatures under vacuum. This strategy eliminates the need for exhaustive protection-deprotection reactions (Marshall, D., L., et. al. 2016, Rapid Commun. Mass Spectrom., 30, 2351-2359) and 20-hydroxyicosyl oleate could be isolated in a 48% yield following column chromatography. The yields observed for the structural analogues were all in the 30-50% range. Synthesis of the corresponding FAHFA, i.e. 20-(oleoyloxy)eicosanoic acid, was achieved by employing a standard Jones oxidation protocol (Balas, L. et. al., 2016, Org. Biomol. Chem., 14, 9012-9020). The isolated yields obtained after extraction and column purification were excellent in this particular reaction, as well as all others performed (70%-quant.).

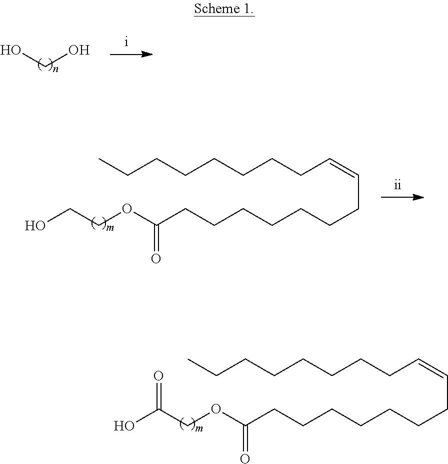

An overview of synthetic route 1 to FAHFAs. i) Oleic acid (1.2 equiv.), corresponding diol (1 equiv.), NaHSO₄·H₂O (3.5 mol %), 100° C., 0.3 mbar, 2.5 h, 40-50% yield. ii) Jones reagent (2.2 equiv.), acetone or acetone:EtOAc:THF mixtures, 0° C. or rt, 0.5-2 h, 70-96% yield.

Oleic acid is shown in Scheme 1 as an example of a suitable fatty acid, and the most abundant acyl chain in the TFLL. Any suitable fatty acid may also be used in this synthetic route. Alternative fatty acids include, in particular, linoleic acid, palmitoleic acid, palmitic acid and stearic acid.

The second synthesis route depicted in Scheme 2 was developed in order to provide access to a large library of TFLL-lipids of increasing complexity. This strategy is based on the use of a block approach featuring a Z-selective Wittig olefination reaction with two distinct fragments: a triphenylphosphonium ylide and an aldehyde. This strategy provides a possibility for tailoring the site of the alkene, the length of the hydrocarbon chain and modification sites/functional groups present etc. In addition, various carboxylic acids can be used in the Steglich-type esterification reaction thus providing ample possibilities for varying the structural features of this fragment as well. Here, this synthetic route will be exemplified by the synthesis of (12Z)-20-oleoyloxyeicos-12-enoic acid (Viitaja, T., et. al. 2021, J. Org. Chem., 86, 4965-4976).

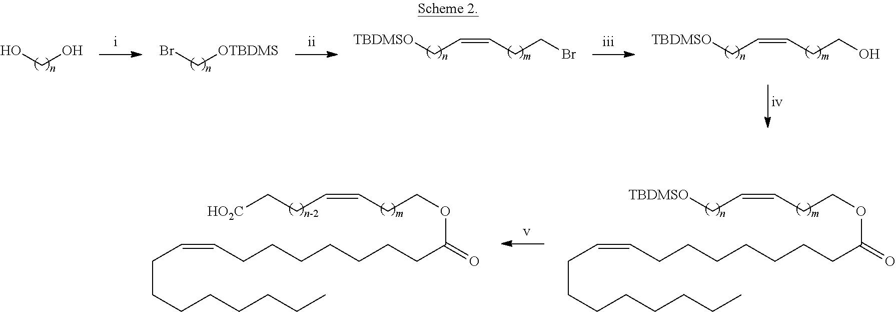

Overview of synthetic route 2 to FAHFAs. i) 1) 48% aq. HBr, cyclohexane, reflux, 18 h, 77%; 2) Imidazole, TBDMSCI, CH₂Cl₂, rt, o/n, 94%; ii) 1) PPh₃, neat, 120° C., 17 h, quant.; 2) NaHMDS, dry THF, HMPA −78° C., 1 h; 8-bromo-octanal (or other brominated aldehyde), −78° C., rt, 24 h, 34%; iii) 1) KOAc, DMSO, 50° C., 27 h, 66%; 2) NaOMe, THF:MeOH (1:2), 22 h, rt, 78%; iv) 1) Oleic acid, DMAP, EDC·HCl, CH₂Cl₂, rt, o/n, 93%; v) 1) TBAF, THF, 1 h, rt, 92%; 2) Jones reagent, acetone:EtOAc (1:1), 0° C., 45 min, 89%.

The reaction started with the successful monobromination of 1,12-dodecanediol in a 77% yield (Greaves, J. et. al. 2017, PNAS, 114, E1365-E1374). The free hydroxyl group was protected as a TBDMS-ether in a 94% yield according to the protocol of Cateni et. al. (Cateni, F. et. al. 2007, Helv. Chim. Acta., 90, 282-290). This product was reacted with 1 equiv. of PPh₃ at 120° C. under neat reaction conditions to give the corresponding triphenylphosphonium bromide salt. The second fragment required for the Wittig olefination reaction, i.e. the monobrominated aldehyde, was obtained by selective oxidation of 8-bromo-1-octanol with PCC. Both of these crude products were carefully dried on the vacuum line and directly used in the subsequent Z-selective Wittig reaction according to a modified literature procedure (Primdahl, K. G. et. al., 2015, Org. Biomol. Chem., 13, 5412-5417). This gave (12Z)-20-Bromo-1-tert-butyldimethylsilyloxyeicos-12-ene in a 34% yield. The direct displacement of the bromide by a hydroxyl group proved challenging and a two-step protocol was devised based on a previously reported protocol (Lee, J. H., et. al. 2008, Korean Chem. Soc., 29, 2491-2495). The bromide was displaced with an acetate anion in a 66% yield and the resulting compound was deacetylated under Zemplan conditions to give (12Z)-20-hydroxy-1-tert-butyldimethylsilyloxyeicos-12-ene in a 77% yield. A Steglich-type esterification reaction employing oleic acid was found to be a smooth protocol for installing the acyl chain. The temporary silyl protective group was at this stage deprotected with 3 eq. of TBAF in excellent yield and the primary alcohol was oxidized to give the representative FAHFA (12Z)-20-oleoyloxyeicos-12-enoic acid in an 89% yield.

The third synthesis route depicted in Scheme 3 was developed in order to provide access to additional structural analogues of TFLL FAHFAs. This strategy is based on the use of a block approach featuring the coupling of an acetylide anion with a monobrominated starting material and requires two distinct fragments: a potentially functionalized hydrocarbon chain containing a terminal alkyne and a potentially functionalized hydrocarbon chain containing an alkyl halide. This synthetic strategy provides a possibility for obtaining an alkyne, selective reduction of the alkyne to either an E or Z-alkene, tailoring the site of the alkyne/alkene, the length of the hydrocarbon chain and modification sites/functional groups present etc. In addition, various carboxylic acids can be used in the Steglich-type esterification reaction thus providing ample possibilities for varying the structural features of this fragment as well. Here, this synthetic route will be exemplified by the synthesis of (21Z)-29-(oleoyloxy)nonacos-21-enoic acid.

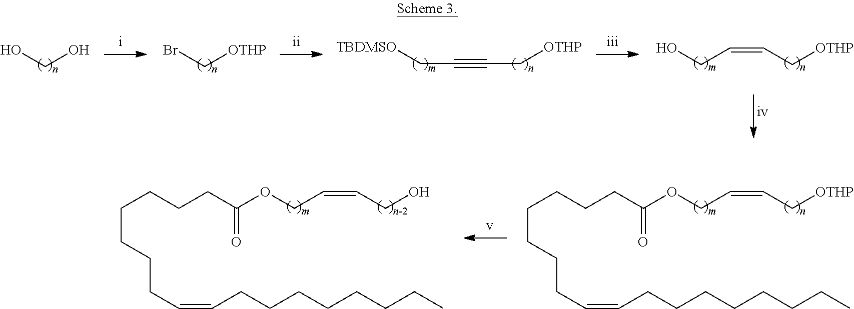

Overview of synthetic route 3 to FAHFAs. i) 1) 48% aq. HBr, cyclohexane, 82° C., 5 h, 52%; 2) DHP, PPTS, CH₂Cl₂, rt, 23 h, 97%; ii) 1) 1-O-tert-butyldimethylsilyl-non-8-yn-1-ol (or analogues compound), HMPA, THF, −78° C., 2) BuLi, 2 h, −40° C.; 3) monobrominated compound, −78° C.; 4) TBAI, rt; 5) 80° C., 20 h; iii) 1) TBAF, THF, rt, 1 h, 52% (over two steps); 2) Lindlar's catalyst, quinoline, benzene, H₂ (1 atm.), rt, 1 h, 89%; iv) Oleic acid, DMAP, EDC·HCl, CH₂Cl₂, rt, 22 h, 76%; v) 1) CSA, MeOH/THF, rt, 18 h, 72%; 2) Jones reagent, acetone/EtOAc, rt, 1 h, 61%. The monobromination was carried out as described above, however, when 1,20-eicosanediol was used as the starting material a notable decrease in the yield was observed (from 77% to 52%). The free hydroxyl group was next protected as a THP-ether in a 97% yield using standard reaction protocols. This marked the successful synthesis of one of the fragments required for the envisioned chain elongation reaction (coupling between an acetylide anion and primary alkyl halide). A suitable alkyne was synthesized from commercially available non-8-ynol by protecting the hydroxyl group as a TBDMS-ether with TBDMSCI and imidazole in CH₂Cl₂. The key conjugation step was performed as follows: the terminal alkyne (2.5 equiv.) was first dissolved in THF:HMPA (3:1) and converted to the acetylide anion at −78° C. with BuLi (2.4 equiv.). 20-Bromo-1-((tetrahydro-2H-pyran-2-yloxy)-eicosanol (1 equiv.) dissolved in THF was then added and the resulting mixture was brought to rt. Finally, a catalytic amount of TBAI was added and the mixture heated at 80° C. o/n. After workup, direct deprotection of the TBDMS-ether was performed with TBAF in THF thereby leading to isolation of 29-(tetrahydro-2H-pyran-2-yloxy)nonacos-8-yn-1-ol in a 52% yield over two steps. The alkyne was selectively reduced to a Z-alkene in an autoclave by employing Lindlar's catalyst and an excess of quinoline, and, using an 1 atm. hydrogen gas pressure with benzene as the solvent. A high yield of 89% was obtained in this reaction step. A striking difference to the earlier literature reports (Hancock, S. E. et. al. 2018, J. Lipid Res., 59, 1510-1518) is that the inventors were able to optimize the reaction conditions thus leading to the isolation of the pure Z-alkene (without notable impurities from the E-isomer). A Steglich-type esterification reaction was once again employed for installing the acyl group, this time in a 76% yield. The THP-ether was next hydrolyzed with CSA in MeOH:THF (3:1) in a 72% yield and the unmasked hydroxyl group was subject to our established Jones oxidation protocol. (21Z)-29-(oleoyloxy)nonacos-21-enoic acid could thereby be isolated in a 61% yield.

As mentioned above, multiple wax esters are commercially available and can be used in combination with FAHFAs in the intended applications. However, the inventors have developed synthetic protocols for TFLL specific wax esters as well. To the best of our knowledge, TFLL specific branched wax esters have not been previously synthesized. Here, two synthetic routes have been developed for the synthesis of wax esters. The first one is a short and efficient route based on the acylation of an alcohol and the second is a lengthier route which enables the synthesis of branched wax esters with further modification possibilities. The methods used in the synthesis are similar to those described above for FAHFAs and similar variations in the structural features are possible e.g. tailoring the hydrocarbon chain lengths, shifting the position of the alkyne/alkene, tailoring the stereochemistry of the alkene functionality, further functionalization of the molecular structures etc. The synthesis will here be exemplified by the synthesis of one straight-chain wax ester and one branched wax ester, namely: hexocosanyl oleate and 24-methylpentacosanyl oleate (see Scheme 4).

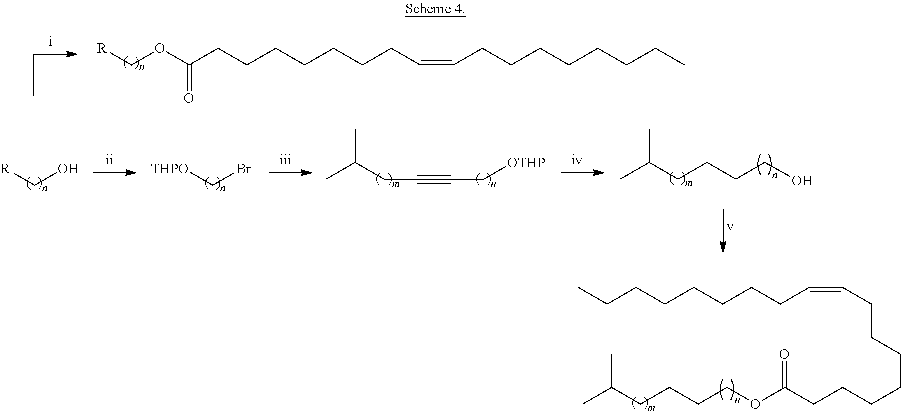

Overview of synthetic routes to noncommercial wax esters. i) oleic acid, DMAP, EDC-HCl, CH₂Cl₂, rt, o/n, 93% (R=H); ii) 1) 48% aq. HBr, cyclohexane, reflux, 6 h, 50-70%; 2) DHP, PPTS, CH₂C2, o/n, 97%; iii) 1) corresponding methyl branched alkyne, BuLi, −78° C. to −40° C., 2 h; 2) corresponding monobrominated THP-ether, −78° C. to rt; 3) TBAI, 10 min. to reflux o/n, 89%; iv) 1) CSA, THF:MeOH (1:3), rt, o/n, 95%; 2) Pd/C (10%), EtOAc, H₂ (6 bar), 4 h, 83%; v) oleic acid, DMAP, EDC-HCl, CH₂Cl₂, rt, o/n, 96%.

The synthesis of hexocosanyl oleate was achieved in one-step by the Steglich-type esterification protocol described above. In more detail, commercial hexacosanol was acylated with oleic acid in a 93% yield.

The synthesis of the branched ester commenced from 1,20-eicosanediol and the monobromination, and, subsequent THP-protection was carried out as described above and in similar yields. This fragment was coupled in an S_{N}2-reaction with the acetylide anion of 4-methylpent-1yne. In more detail, 4-methylpent-1-yne was deprotonated with BuLi at −78° C. and then allowed to react with 20-bromo-1-(tetrahydro-2H-pyran-2-yloxy)-eicosanol and TBAI at elevated temperatures. This afforded the hydrocarbon base in 89% yield. Deprotection of the THP ether with CSA in MeOH:THF 3:1 followed by reduction of the triple bond by hydrogenation gave the branched alcohol in a 79% overall yield. Acylation of the alcohol with oleic acid gave 24-methylpentacosanyl oleate in a 96% yield.

### 2.2 Characterization of Functioning Principle

### Background

In order to effectively retard evaporation from the ocular surface, the TFLL must fulfill two criteria: 1) the lipids need to spread rapidly and cover the entire aqueous tear film surface as the eye is opened, and, 2) the film formed by the lipids needs to have a condensed structure that prevents or retards the passage of water molecules through it. These same two requirements need to be fulfilled by a lipid composition developed for retarding or preventing the evaporation of water if the approach is intended to provide an answer to the crucial tear film instability defect or sustain water in artificial lakes and water reservoirs. Thus, the inventors have focused on providing insights on these two factors, i.e. 1) spreading behavior and 2) evaporation resistance, and, characterizing the underlying functioning principle and requirements from the composition point-of-view.

The inventors have previously reported on the properties of pure OAHFAs and wax esters. In more detail, they showed that at physiological temperatures and low surface pressures, pure OAHFAs (or their structural analogues such as O-acyl-o-hydroxy fatty alcohols) form a monolayer with the molecules lying flat on the aqueous surface. (Bland, H., C., et al. 2019, Langmuir, Vol. 35, 3545-3552; Schuett, B., S. et al. 2013, Exp Eye Res, Vol. 115, 57-64) As the surface pressure was increased, the OAHFA molecules gradually adopted an upright orientation with the polar headgroup (carboxylic acid in the case of OAHFAs) pointing toward the water phase, whereas the ester group turned away from the water phase. (Bland, H., C., et al. 2019, Langmuir, Vol. 35, 3545-3552; Schuett, B., S. et al. 2013, Exp Eye Res, Vol. 115, 57-64) Long-chained OAHFAs additionally underwent a transition to a condensed monolayer phase, which was accompanied by an evaporation resistance of up to 5 s/cm. (Bland, H., C., et al. 2019, Langmuir, Vol. 35, 3545-3552) Wax esters also spread to form a monolayer on aqueous surfaces when the temperature of the aqueous phase was close to or higher than the melting point of the wax ester. (Rantamski, A., H. et al. 2013, Invest Ophthalmol Vis Sci, 54, 5211-5217; Paananen, R., O. et. al. 2014, Langmuir, 30, 5897-5902; 2019, J. Phys. Chem. Lett., 10, 3893-3898). At lower temperatures, spreading of wax esters is not observed. (Rantamski, A., H. et al. 2013, Invest Ophthalmol Vis Sci, 54, 5211-5217; Paananen, R., O. et. al. 2014, Langmuir, 30, 5897-5902; 2019, J. Phys. Chem. Lett., 10, 3893-3898) Thus, wax esters can form a solid film that resists evaporation but this is restrained to a marginal temperature range close to the melting point of the wax ester. (Rantamski, A., H. et al. 2013, Invest Ophthalmol Vis Sci, 54, 5211-5217; Paananen, R., O. et. al. 2014, Langmuir, 30, 5897-5902; 2019, J. Phys. Chem. Lett., 10, 3893-3898). To summarize, the marginal temperature range under which wax esters could be applied in the intended applications places severe constraints on their practical use. In addition, the maximum evaporation resistance achieved with wax esters alone is poor (2-3 s/cm) and our studies on pure OAHFAs have showed that these are only marginally better (˜5 s/cm). Thus, neither OAHFAs nor wax esters display optimal properties on their own.

### OAHFA and Wax Ester Mixtures

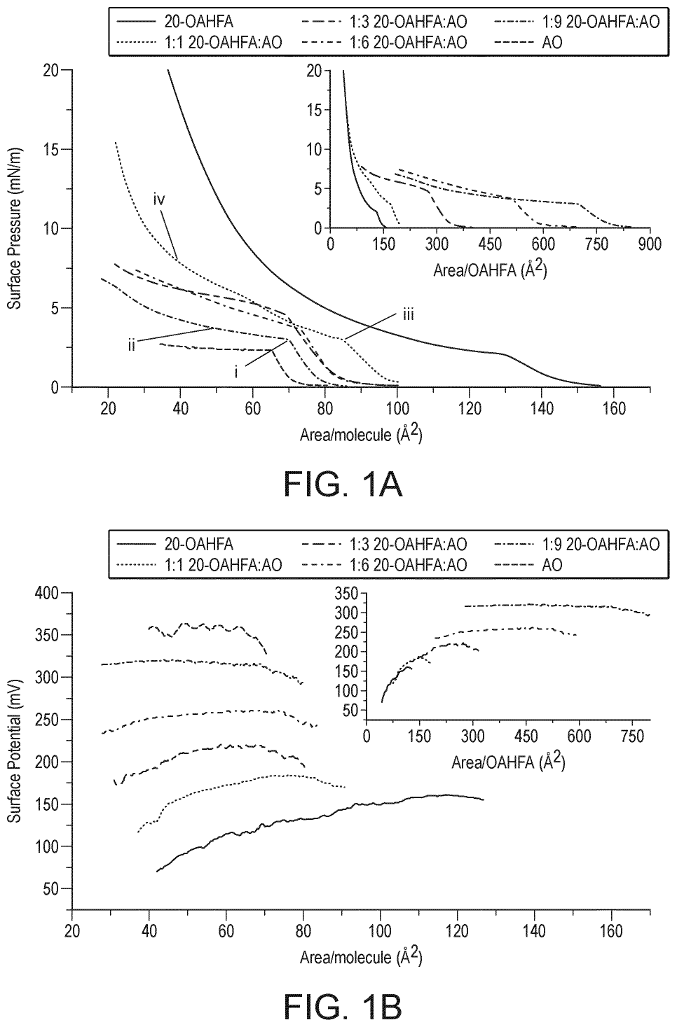

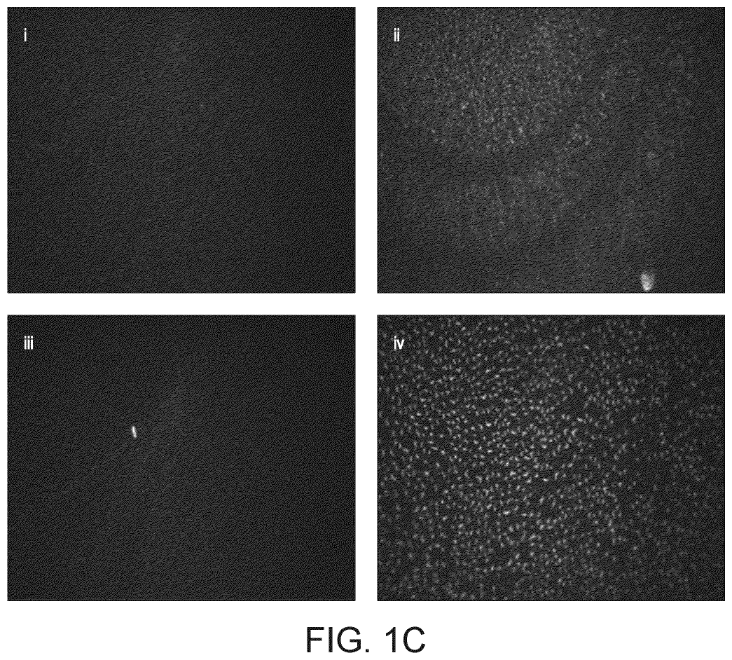

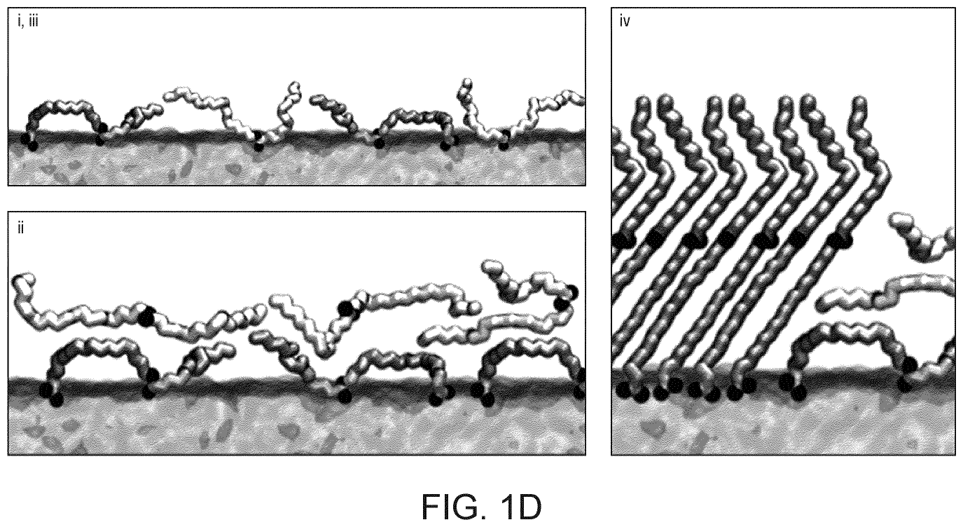

Mixtures of OAHFAs and wax esters display more complex surface behavior which has not been extensively studied previously. In addition, the surface behavior varies greatly depending on the individual components utilized. For example, the inventors have previously showed that the use of OAHFA and cholesteryl ester mixtures does not lead to improved properties over OAHFAs alone (Paananen et. al. Ocul. Surf. 2020). Thus, the mixtures of OAHFAs/FAHFAs and wax esters are unique in this regard as will be further highlighted in the following discussion. Two examples will be given herein, the first example involves the use of 20-OAHFA and AO mixtures (see FIG. 1).

The organizational behavior of FAHFA/OAHFA and wax ester mixtures were investigated by measuring surface pressure and surface potential isotherms, and, imaging the monolayer structure with a Brewster angle microscope. A phosphate buffered saline (PBS) solution was used as an in vitro model of the ocular surface and the measurements were conducted at physiological temperature (35° C.). With this model system, it is possible to accurately simulate the conditions residing on the ocular surface and the changes occurring during a blink of the eye, both in terms of surface pressure and the capabilities of the monolayer to respread over many compression-expansion cycles. Thus, the study protocol provides the essential information required for evaluating the suitability of the composition in the intended applications. More details on the study protocol can be found in the inventors' previous publications on the topic (Paananen, Ekholm et. al. in Langmuir 2019, Ocul. Surf. 2020, J. Org. Chem. 2021).

In pure form, both 20-OAHFA and AO form liquid monolayers upon spreading with surface pressure lift-offs occurring at 140 Å²/molecule and 70 Å²/molecule, respectively. The difference in mean molecular area reflects the different orientation of molecules at the surface. The 20-OAHFA lays flat on the aqueous surface and AO adopts an orientation in which the ester group is only weakly coordinated toward the interface (Bland, H., C., et al. 2019, Langmuir, Vol. 35, 3545-3552; Paananen, R., O. et. al. 2019, J. Phys. Chem. Lett., 10, 3893-3898). 20-OAHFA undergoes a transition to a solid monolayer at 2 mN/m surface pressure accompanied by a conformational change to an upright molecular orientation (FIG. 1D: iv) as seen from the surface pressure plateau and the decrease in surface potential as reported previously (Bland, H., C., et al. 2019, Langmuir, Vol. 35, 3545-3552). In contrast, the AO film collapsed at a similar surface pressure.

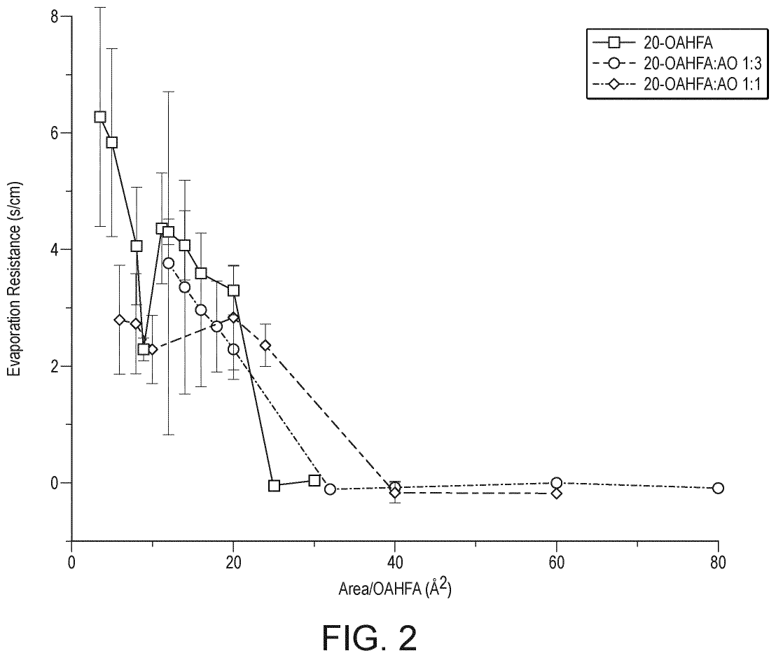

In mixed films, 20-OAHFA and AO were ideally miscible in the liquid monolayer phase, indicated by the fact that the mean molecular area and surface potential at surface pressure lift-off were directly proportional to the ratio of the two components (FIG. 1A, 2B). Upon compression of the films, the AO collapsed as droplets on top of the monolayer at low surface pressures (3-5 mN/m) as indicated by bright droplets in the BAM images (FIG. 1C: ii) and a plateau in the surface pressure and potential isotherms (FIG. 1A, 2B). The 20-OAHFA left in the monolayer formed a solid monolayer phase (FIG. 1C: iii), unaffected by the presence of AO, which can be deduced by analyzing the surface pressure and potential isotherms per molecule of 20-OAHFA (FIG. 1A, 1B). The presence of AO did not affect the evaporation resistance of 20-OAHFA:AO mixtures (FIG. 2), as the evaporation resistance was found to be completely dependent on the adaptation of a solid phase by the 20-OAHFA, and a maximum evaporation resistance of 3-5 s/cm was observed which is in line with the values obtained for pure 20-OAHFA. (Bland, H., C., et al. 2019, Langmuir, Vol. 35, 3545-3552). The evaporation resistance of 20-OAHFA:AO-mixtures is shown as a function of area/OAHFA in FIG. 2.

Thus, a mixture of 20-OAHFA and AO does not provide additional benefits over the use of an OAHFA, or a structural analogue, on its own.

The following example will focus on the properties of 20-OAHFA and BO mixtures. The only structural difference between AO and BO is an increase in the alkyl chain length by two carbon atoms and the only deviation in terms of chemical properties is the slightly higher melting/boiling point of BO. From the biophysical perspective, this is considerable since AO is in the liquid state at physiological conditions while BO is in the solid state. Because of this factor, reproducible isotherms could not be obtained with pure BO, as it did not spread on the aqueous surface but formed solid aggregates instead. This confirms that BO would not be suitable on its own in the intended applications as it does not fulfill the crucial criteria discussed in the beginning of this section.

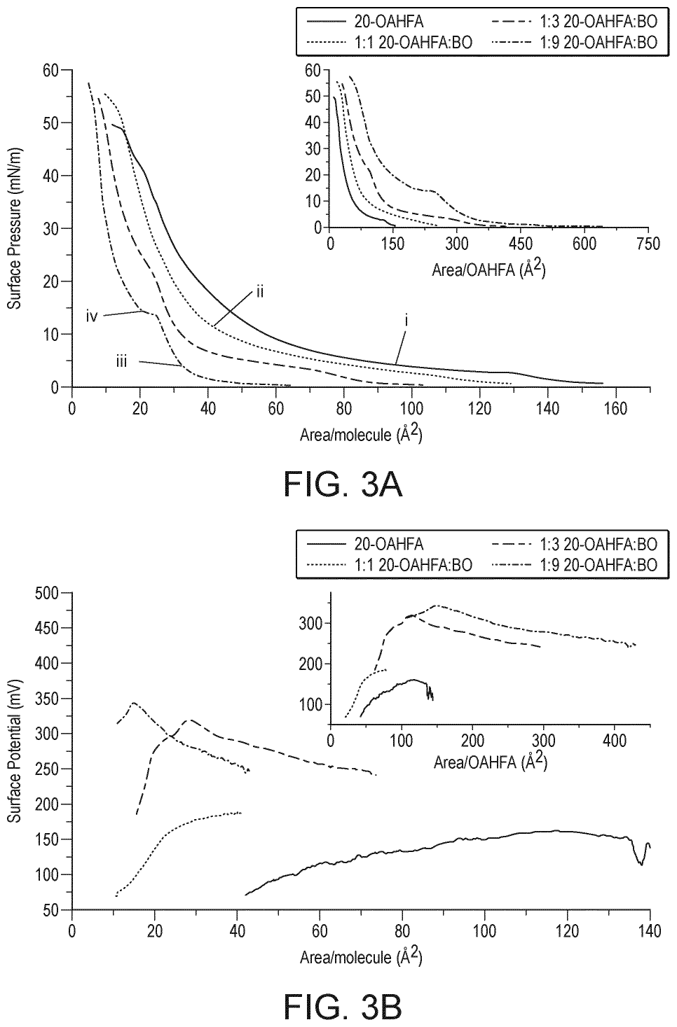

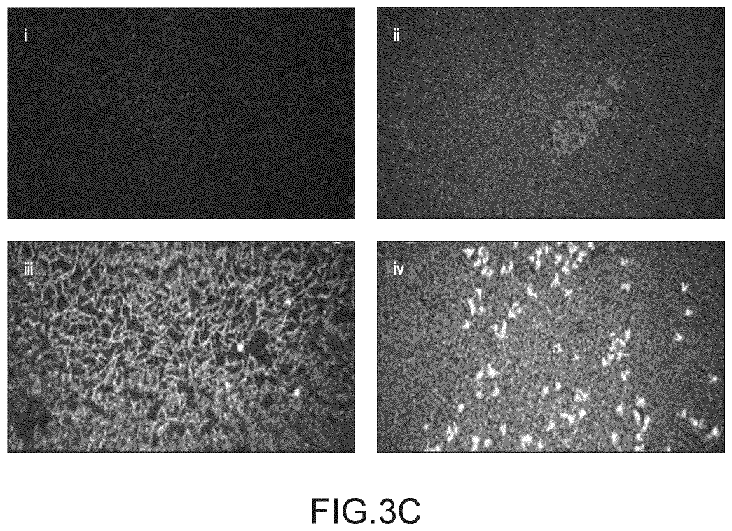

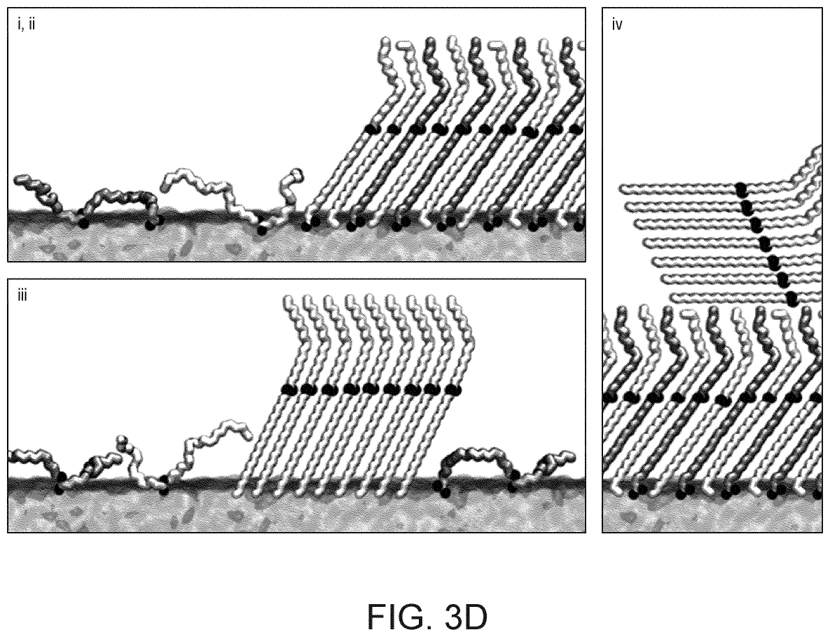

This being said, BO and 20-OAHFA mixtures containing up to 50 mol-% of BO forms a stable homogeneous monolayer (FIG. 3C: i), showing the cooperative action of appropriately designed FAHFA/OAHFA and wax ester mixtures. In this case, the 20-OAHFA induces spreading of BO and the formation of a miscible liquid monolayer at low surface pressures. During compression of the 1:1 mixture, a liquid to solid phase transition (FIG. 3C; ii) occurred, accompanied by a steep increase in surface pressure up to 40 mN/m at a mean molecular area of ˜20 Å²/molecule (FIG. 3A). In contrast to the previous example, BO was found to integrate into the condensed monolayer phase instead of collapsing on top of the polar lipid layer. At higher BC-ratios (>50 mol-%), only a set fraction of the BO was incorporated into the homogenous amphiphilic monolayer while the rest formed solid aggregates on top of it as observed from the BAM-images (FIG. 3C; iii). The solid aggregates persisted even in a compressed state (FIG. 3C; iv). In mixtures containing more than 50 mol-% BO, a second plateau was apparent in the surface pressure isotherms at 17-22 mN/m followed by a steep increase at mean molecular areas of 40 Å²/OAHFA (FIG. 3A). This proved that an approximate 1:1 mixture of 20-OAHFA and BO remained at the interface, while the rest of BO formed solid aggregates on top of it during the second plateau. In a similar fashion, the steep decrease in the surface potential isotherm was in line with the values observed for the 1:1 mixture indicating that the condensed sublayer consists of approximately a 1:1 mixture of BO and 20-OAHFA (FIG. 3B).

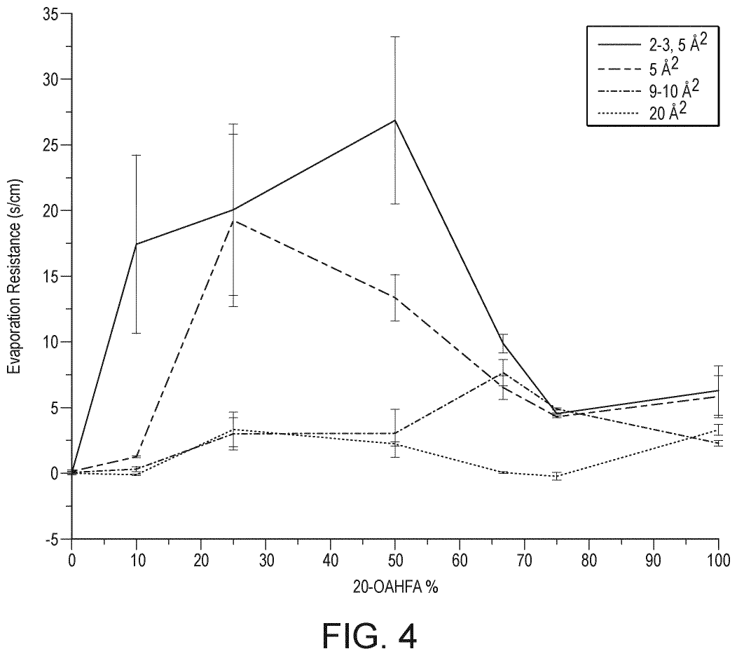

The rapid and integrative self-assembly and cooperative spreading behavior of 20-OAHFA and BO mixtures was interesting and would undoubtedly have an impact on the important evaporation resistant property of the monolayer as this was no longer solemnly dependent on the amphiphilic OAHFA/FAHFA molecules residing at the aqueous interface. The evaporation resistance of the mixed condensed monolayer phase formed by 20-OAHFA and BO was studied using the methodology described in Section 1.2.4 for various ratios of the two components. The integration of BO into the 20-OAHFA monolayer drastically improved the evaporation resistance from 3-5 s/cm observed for pure 20-OAHFA to up to 25 s/cm (FIG. 4). The inventors screened the evaporation resistance of a number of 20-OAHFA:BO mixtures (examples 3:1, 2:1, 3:2, 1:1, 1:3, 1:9 included herein). In mixtures containing less than 50 mol-% of BO, the maximum evaporation resistance achieved was only marginally improved over pure 20-OAHFA. In contrast, mixtures containing 50 mol-%, or more, of BO, had considerable improved evaporation resistance, showing that the increased evaporation resistance is caused by formation of a mixed monolayer of 20-OAHFA and BO as described above. Evaporation resistance of 20-OAHFA:BO mixtures was also significantly greater than the estimates reported for the natural TFLL (9-13 s/cm) (Iwata, S., et al. 1969, Invest Ophthalmol Vis Sci, 8, 613-619; Peng, C., et al. 2014, Ind Eng Cham Res, 53, 18130-18139). To the best of our knowledge, the evaporation resistance of these mixtures surpasses all previously reported strategies in the scientific and patent literature (Barnes, G. T., 2008, Agric. Water. Manage., 95, 339-353).

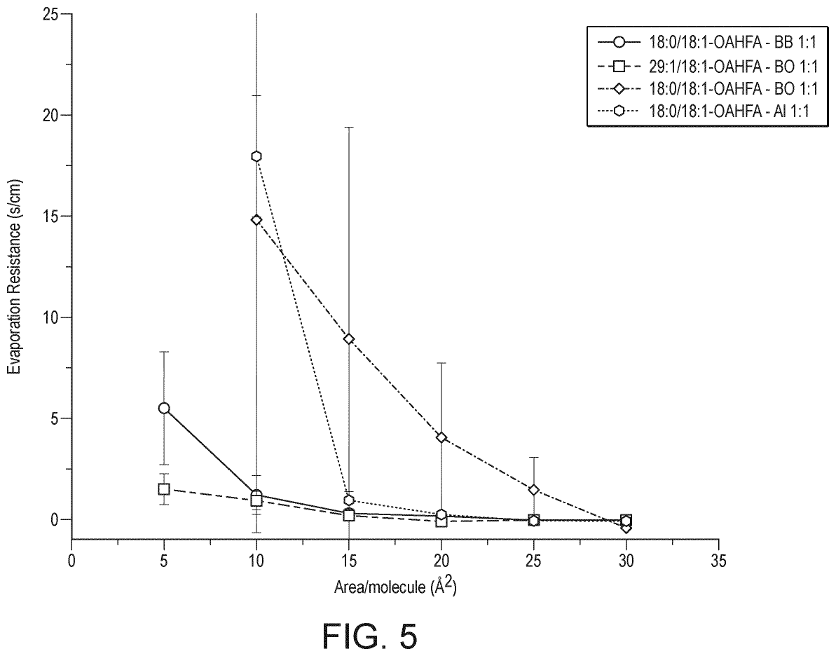

The evaporation resistance of 1:1 mixtures of 18-(oleoyloxy)stearic acid (18:0/18:1-OAHFA) and behenyl oleate (BO), (18:0/18:1-OAHFA:B:O 1:1), (21Z)-29-(oleoyloxy)nonacos-21-enoic acid (29:1/18:1-OAHFA) and BO (29:1/18:1-OAHFA:B:O 1:1), 18:0/18:1-OAHFA and behenyl behenoate (BB), and, 18:0/18:1-OAHFA and arachidyl laurate (AL) was also investigated using the methodology described in Section 1.2.4. Measurements were taken at areas per molecule of 5 to 35 Å² and the evaporation resistance for each mixture was measured in s/cm. The results of the experiment are shown in FIG. 5.

As can be seen from FIG. 5, the 1:1 mixtures of 18:0/18:1-OAHFA and BO, and 18:0/18:1-OAHFA and AL displayed improved evaporation resistant properties compared to the individual components, indicating that these combinations also form a tightly packed layer at the aqueous surface and therefore that improved evaporation resistance can be achieved by different combinations of OAHFAs and wax esters. In particular, very high evaporation resistance was observed for both of these combinations at an area per molecule of 10 Å². Even higher evaporation resistance was observed at lower areas per molecule, but the exact value was not measurable with the experimental set-up available. However, the 1:1 mixtures of 18:0/18:1-OAHFA and BB and mixtures of 29:1/18:1-OAHFA and BO, did not display improved evaporation resistant properties over those of the OAHFA alone. Therefore, it is clear that the identities of both the OAHFA and the wax ester, and how they interact in combination, are important for evaporation resistance. Moreover, 18:0/18:1-OAHFA has been shown to form highly evaporation resistant mixtures with BO, which is a solid under physiological conditions, and AL which is a liquid under these conditions, showing that the effect cannot easily be predicted from the melting points of the wax ester. Similarly, the melting point of the OAHFA does not appear to be the main determining factor in the behaviour of the mixture as the combination of 29:1-OAHFA and BO did not display improved properties although the difference between the melting points of 18-OAHFA and 29:1-OAHFA is only around 3° C. (56° C. vs. 53° C.)

The inventors here show that appropriately designed mixtures of FAHFAs/OAHFAs and wax esters spread rapidly at an aqueous surface under ambient conditions and that the condensed monolayer formed has a remarkable evaporation resistance. These unique biophysical properties stem from the intrinsic nature of appropriately designed FAHFA/OAHFA and wax ester mixtures alone. Therefore, it is clear that such mixtures can be applied in multiple applications where an increased evaporation rate of water represents a challenge. Application areas span from the treatment of ocular surface diseases to the sustenance of water in artificial lakes and reservoirs, and, beyond.

### 3. Conclusions

The tear film consists of two distinct layers, the aqueous layer and the TFLL. It is widely accepted that the TFLL has a central role in stabilizing the tear film. Over the years, research by the inventors and others has been piling up to suggest that the TFLL stabilizes the tear film by acting as a barrier to evaporation of water from the underlying aqueous layer (Paananen, R., O. et. al. 2014, Langmuir, 30, 5897-5902; 2019, J. Phys. Chem. Lett., 10, 3893-3898; 2020, Ocul. Surf., 18, 545-553; Craig, J., P. et. al. 1997, Optom. Vis. Sci., 8, 613-619; King-Smith, P. E., et. al. 2010, Invest. Ophthalmol. Vis. Sci., 51, 2418-2423; Dursh, T., J. et. al. 2018, Optom. Vis. Sci., 95:5). The loss of this evaporation resistant function leads to drying of the eyes in the majority of DED-cases, which may further cause inflammation and ocular surface damage. In order to effectively retard evaporation from the ocular surface, the TFLL must fulfill two criteria: 1) the lipids need to spread rapidly and cover the entire aqueous tear film surface as the eye is opened, and, 2) the film formed by the lipids needs to have a condensed structure that prevents or retards the passage of water molecules through it. These same two criteria need to be fulfilled for a DED-treatment aimed at replenishing the anti-evaporative properties of the TFLL and targeting the crucial tear film instability defect.

The inventors have developed synthetic protocols for the synthesis of an extensive library of TFLL FAHFAs and wax esters, and, their structural analogues. Using this library, the inventors have identified mixtures of the key lipid species that combine exceptionally high evaporation resistance with effective spreading on the aqueous interface, i.e. a composition which fulfills the two criteria described above. Importantly, the core technology of the inventors is based on a tailored synthetic lipid composition with few variables and the synthetic tools required to produce the individual components and a wide range of structural analogues have been established. The inventors have invested effort in mapping the molecular level mechanism of their core technology with experimental biophysical techniques.

The result of the development process is a core composition of FAHFAs and wax esters which forms an evaporation resistant barrier on an aqueous interface under physiological conditions. The inventors have shown that the mixtures provide superior properties over the pure components on their own. The evaporation resistance noted exceeds the highest evaporation resistance values reported in the literature for any other type of compound, mixture or product (Barnes, G. T., 2008, Agric. Water. Manage., 95, 339-353). Administering this lipid composition on the ocular surface represents a unique and highly promising treatment option for DED and/or eye discomfort. Moreover, because the biophysical properties stem from the intrinsic properties of the mixture as such and are not directly linked to the surrounding environment—these mixtures can likewise be used to prevent the evaporation of water from materials and other surroundings e.g. artificial lakes and water reservoirs. The latter task represents an increasing challenge due to the global climate change.

1. A composition comprising a combination of a fatty acid ester of a hydroxy fatty acid (FAHFA) or a structural analogue thereof and a wax ester or a structural analogue thereof, and optionally one or more additives.

2. The composition of claim 1, wherein the composition consists of a combination of a FAHFA or a structural analogue thereof and a wax ester or a structural analogue thereof, and optionally one or more additives.

3. The composition of claim 1, wherein the composition is a pharmaceutical composition.

4. The composition of claim 1, wherein the evaporation resistance of the composition is more than 1 s/cm, more than 2 s/cm or more than 3 s/cm, more than 5 s/cm, more than 9 s/cm, more than 10 s/cm, more than 13 s/cm, more than 15 s/cm, more than 20 s/cm, more than 25 s/cm or more than 30 s/cm, preferably wherein the evaporation resistance of the composition is more than 20 s/cm.

5. The composition of claim 1, wherein the FAHFA is an O-Acyl-ω-hydroxy fatty acid (OAHFA).

6. The composition of claim 1, wherein the FAHFA and/or OAHFA is selected from the group consisting of oleic acid based fatty acid esters, palmitoleic acid based fatty acid esters, myristoleic acid based fatty acid esters, lauric acid based fatty acid esters, paullinic acid based fatty acid esters, gondoic acid based fatty acid esters, erucic acid based fatty acid esters, nervonic acid based fatty acid esters, linoleic acid based fatty acid esters and linolenic acid based fatty acid esters; and/or the structural analogue of a FAHFA are selected from the group consisting of oleic acid-based alcohols, palmitoleic acid-based alcohols, myristoleic acid-based alcohols, lauric acid-based alcohols, paullinic acid-based alcohols, gondoic acid-based alcohols, erucic acid-based alcohols, nervonic acid-based alcohols, linoleic acid based alcohols and linolenic acid based alcohols.

7. The composition of claim 1, wherein the FAHFA is selected from the group consisting of 18-(oleoyloxy)stearic acid (18:0/18:1-OAHFA; 18-OAHFA), 12-(linoleoyloxy)dodecanoic acid, 20-(linoleoyloxy)eicosanoic acid, 12-(palmitoleoyloxy)dodecanoic acid, 20-(palmitoleoyloxy)eicosanoic acid, 12-(palmitoyloxy)dodecanoic acid, 20-(palmitoyloxy)eicosanoic acid, 12-(stearoyloxy)dodecanoic acid, 20-(stearoyloxy)eicosanoic acid, 12-OAHFA (12-(oleoyloxy)dodecanoic acid), 15-OAHFA (15-(oleoyloxy)pentadecanoic acid), 20-OAHFA (20-(oleoyloxy)eicosanoic acid), 22-OAHFA (22-(oleoyloxy)docosanoic acid), 20:1-OAHFA ((12Z)-20-(oleoyloxy)eicos-12-enoic acid) and 29:1-OAHFA ((21Z)-29-(oleoyloxy)nonacos-21-enoic acid), optionally wherein the FAHFA is selected from the group consisting of 18-(oleoyloxy)stearic acid (18:0/18:1-OAHFA; 18-OAHFA), 12-OAHFA (12-(oleoyloxy)dodecanoic acid), 15-OAHFA (15-(oleoyloxy)pentadecanoic acid), 20-OAHFA (20-(oleoyloxy)eicosanoic acid), 22-OAHFA (22-(oleoyloxy)docosanoic acid), 20:1-OAHFA ((12Z)-20-(oleoyloxy)eicos-12-enoic acid) and 29:1-OAHFA ((21Z)-29-(oleoyloxy)nonacos-21-enoic acid)

8. The composition of claim 1, wherein the FAHFA is selected from the group consisting of 12-OAHFA (12-(oleoyloxy)dodecanoic acid), 15-OAHFA (15-(oleoyloxy)pentadecanoic acid), 20-OAHFA (20-(oleoyloxy)eicosanoic acid), 22-OAHFA (22-(oleoyloxy)docosanoic acid), 20:1-OAHFA ((12Z)-20-(oleoyloxy)eicos-12-enoic acid) and 29:1-OAHFA ((21Z)-29-(oleoyloxy)nonacos-21-enoic acid).

9. The composition of claim 1, wherein the FAHFA is selected from the group consisting of 20-(oleoyloxy)eicosanoic acid and 18-(oleoyloxy)stearic acid; optionally wherein the FAHFA is 20-(oleoyloxy)eicosanoic acid.

10. The composition of claim 1, wherein the wax ester is selected from the group consisting of n-oleic acid based esters, iso-branched alkyl oleates, anteiso-branched alkyl oleates, palmitoleic acid based esters, iso-branched alkyl palmitoleates, anteiso-branched alkyl palmitoleates, myristoleic acid based esters, iso-branched alkyl myristoleates, anteiso-branched alkyl myristoleates, lauric acid based esters, iso-branched alkyl laurates, anteiso-based alkyl laurates, paullinic acid based esters, iso-branched alkyl paullinate, anteiso-branched alkyl paullinate, gondoic acid based esters, iso-branched alkyl gondoates, anteiso-based alkyl gondoates, erucic acid based esters, iso-branched alkyl eruciates, anteiso-branched alkyl eruciates, nervonic acid based esters, iso-branched alkyl nervonatess, anteiso-branched nervonate, linoleic acid based esters, iso-branched alkyl linoleates, anteiso-branched alkyl linoleates, linolenic acid based esters, iso-branched alkyl linolenates, and anteiso-branched alkyl linolenates.

11. The composition of claim 1, wherein the wax ester is selected from the group consisting of palmityl oleate, stearyl oleate, arachidyl oleate (AO), behenyl oleate (BO), lignoceryl oleate, hexocosanyl oleate, 24-methylpentacosanyl oleate, palmityl palmitoleate, stearyl palmitoleate, arachidyl palmitoleate, behenyl palmitolate, lignoceryl palmitoleate, 24-methylpentacosanyl palmitoleate, palmityl linoleate, stearyl linoleate, arachidyl linoleate, behenyl palmitolate, lignoceryl linoleate, 24-methylpentacosanyl linoleate, palmityl linolenate, stearyl linolenate, arachidyl linolenate, behenyl palmitolate, lignoceryl linolenate, and 24-methylpentacosanyl linolenate.

12. The composition of claim 1, wherein the wax ester is behenyl oleate or arachidyl laurate; optionally wherein the wax ester is behenyl oleate.

13. The composition of claim 1, wherein the wax ester or structural analogue thereof is in the liquid state at the physiological conditions, and/or the wax ester or structural analogue thereof are in the solid state at the physiological conditions.

14. The composition of claim 1, wherein the molar ratio of the FAHFA or structural analogue thereof to wax ester and/or or structural analogue thereof is 1:1 or less, about 1:1 to 1:100, or 1:2, 1:3, 1:4, 1:5, 1:6, 1:7, 1:8, 1:9 or 1:10, 1:20, 1:30, 1:40, 1:50, 1:60, 1:70, 1:80, 1:90.

15. The composition of claim 1, wherein the molar ratio of the FAHFAs or structural analogue thereof to wax ester or structural analogues thereof, is more than 1:1, about 100:1 to 1:1, about 3:2 to 5:1 or 90:1, 80:1, 70:1, 60:1, 50:1, 40:1, 30:1, 20:1, 10:1, 9:1, 8:1, 7:1, 6:1, 5:1, 4:1, 3:1, or 2:1.

16. The composition of claim 1, wherein the molar ratio of the FAHFA or structural analogue thereof to the wax ester or structural analogue thereof is from 1:1 to 1:9 (FAHFA or structural analogue thereof:wax ester or structural analogue thereof).

17. The composition of claim 1, wherein the molar ratio of the FAHFA or structural analogue thereof to the wax ester or structural analogue thereof is about 1:1 (FAHFA or structural analogue thereof:wax ester or structural analogue thereof).

18. The composition of claim 1, wherein the FAHFA is selected from the group consisting of 20-(oleoyloxy)eicosanoic acid and 18-(oleoyloxy)stearic acid and the wax ester is selected from the group consisting of behenyl oleate and arachidyl laurate.

19. The composition of claim 1, wherein the FAHFA is 20-(oleoyloxy)eicosanoic acid and the wax ester is behenyl oleate; optionally wherein the ratio of 20-(oleoyloxy)eicosanoic acid to behenyl oleate is 1:1 or less, such as from 1:1 to 1:9.

20. The composition of claim 1, wherein the FAHFA is 18-(oleoyloxy)stearic acid and the wax ester is selected from the group consisting of behenyl oleate and arachidyl laurate; optionally wherein the ratio of 18-(oleoyloxy)stearic acid to wax ester is 1:1 or less, such as about 1:1.

21. The composition of claim 1, wherein the one or more additives are selected from the group consisting of solvents, diluents, carriers, buffers, excipients, adjuvants, carrier media, antiseptics, fillers, stabilizers, thickening agents, emulsifiers, disintegrants, lubricants, and binders, and any combination thereof.

22. The composition of claim 21, wherein the one or more pharmaceutically acceptable excipients are ophthalmologically acceptable excipients selected from the group consisting of polyethylene glycol, propylene glycol, glycerin, polyvinyl alcohol, povidone, polysorbate 80, hydroxypropyl methylcellulose, carmellose, carbomer 980, sodium hyaluronate and dextran.

23. The composition of claim 1, wherein the composition comprises at least 0.001 wt %, 0.005 wt %, 0.01 wt %, 0.05 wt %, 0.1 wt %, 0.5 wt %, 1 wt %, 2 wt %, 3 wt %, 4 wt %, 5 wt %, 6 wt %, 7 wt %, 8 wt %, 9 wt %, 10 wt %, 15 wt %, 20 wt %, 25 wt %, 30 wt %, 35 wt %, 40 wt %, 45 wt %, or 50 wt % of the FAHFA or structural analogue thereof, and wax ester or structural analogue thereof.

24. The composition of claim 1, wherein the composition is in a liquid, semisolid or solid form; the composition is in a form of a solution, emulsion, suspension, spray, powder, tablet, pellet, or capsule; or the composition is an oil-in-water emulsion.

25. A composition comprising a combination of:
i. an O-Acyl-o-hydroxy fatty acid, selected from the group consisting of 20-(oleoyloxy)eicosanoic acid, 18-(oleoyloxy)stearic acid and 20-(palmitoleoyloxy)eicosanoic acid; and
ii. a wax ester, selected from the group consisting of behenyl oleate and arachidyl laurate;
wherein the ratio of the O-Acyl-o-hydroxy fatty acid to wax ester is 1:1 or less, such as from 1:1 to 1:9 (O-Acyl-o-hydroxy fatty acid:wax ester).

26. The composition as claimed in claim 25 wherein the O-Acyl-o-hydroxy fatty acid is 20-(oleoyloxy)eicosanoic acid and the wax ester is behenyl oleate.

27. The composition as claimed in claim 25, wherein the O-Acyl-o-hydroxy fatty acid is 18-(oleoyloxy)stearic acid and the wax ester is selected from the group consisting of behenyl oleate and arachidyl laurate; optionally wherein the ratio of the O-Acyl-o-hydroxy fatty acid:wax ester is about 1:1.

28. A composition as defined in claim 1, for use as a medicament.

29. A composition as defined in claim 1, for use in the treatment of dry eye disease and/or Meibomian gland dysfunction, or for use in alleviation of eye discomfort.

30. A method of preparing a composition as defined in claim 1, wherein the method comprises combining or mixing one or more FAHFAs or structural analogues thereof and one or more wax esters or structural analogues thereof, and optionally one or more additives.

31. The method of claim 30, wherein the method further comprises preparing or synthesizing the FAHFA or a structural analogue thereof before mixing it with the wax ester or a structural analogue thereof; and/or preparing or synthesizing the wax ester or a structural analogue thereof before mixing it with the FAHFA or a structural analogue thereof.

32. A non-therapeutic or therapeutic method of preventing evaporation of water, wherein the method comprises applying a composition as defined in claim 1 on a surface to be protected from evaporation or to a material to be protected from evaporation.

33. Use of a composition of as defined in claim 1, for preventing evaporation of water.

34. A method of treating dry eye disease and/or Meibomian gland dysfunction, or alleviating eye discomfort, wherein the method comprises administering a composition as defined in claim 1, to the surface of an eye of a subject in need thereof.
